(19) United States
(12) Patent Application Publication
(10) Pub. No.: US 2023/0226195 A1
(43) Pub. Date: Jul. 20, 2023

(54) TARGETED ABERRANT ALPHA-SYNUCLEIN SPECIES AND INDUCED UBIQUITINATION AND PROTEOSOMAL CLEARANCE VIA CO-RECRUITMENT OF AN E3-LIGASE SYSTEM

(71) Applicants: DANA-FARBER CANCER INSTITUTE, INC., Boston, MA (US); THE GENERAL HOSPITAL CORPORATION, Boston, MA (US)

(72) Inventors: Fleur M. Ferguson, Cambridge, MA (US); Nathanael S. Gray, Stanford, CA (US); Tinghu Zhang, Brookline, MA (US); Stephen J. Haggarty, Gloucester, MA (US)

(73) Assignees: DANA-FARBER CANCER INSTITUTE, INC., Boston, MA (US); THE GENERAL HOSPITAL CORPORATION, Boston, MA (US)

(21) Appl. No.: 18/010,695

(22) PCT Filed: Jun. 16, 2021
(86) PCT No.: PCT/US2021/037551

§ 371 Date: Dec. 15, 2022

Related U.S. Application Data
(60) Provisional application No. 63/040,105, filed on Jun. 17, 2020.

(51) Int. Cl. A61K 47/54

(52) U.S. Cl. CPC A61K 47/545

(57) ABSTRACT

Disclosed are bispecific compounds (degraders) that target α-synuclein protein for degradation. Also disclosed are pharmaceutical compositions containing the degraders and methods of using the compounds to treat neurodegenerative diseases.

### RELATED APPLICATIONS

This application claims the benefit of priority under 35 U.S.C. § 119(e) to U.S. Provisional Application No. 63/040,105, filed Jun. 17, 2020, which is incorporated herein by reference in its entirety.

### BACKGROUND OF THE INVENTION

α-Synuclein protein, encoded by the SNCA gene, is primarily found in the brain (Stefanis, L., Cold Spring Harb. Perspect. Med. 2(2):a009399 (2012)). It is the primary structural component of Lewy bodies, the pathological hallmark of Parkinson's disease (Meade et al., Mol. Neurodegener. 14(1):29 (2019)). These α-synuclein aggregates are thought to contribute to disease development and progression. Currently, α-synuclein antibodies and vaccines are undergoing clinical trials, with the goal of promoting α-synuclein clearance in patients (Zella et al., Neurol. Ther., 8(1):29-44 (2019)). However, no small molecule therapies have been reported that promote the clearance of α-synuclein. Improved therapeutic modalities are needed in order to overcome these challenges for combating aberrant α-synuclein species in clinical settings.

### SUMMARY OF THE INVENTION

A first aspect of the present invention is directed to a bispecific compound of formula (I),

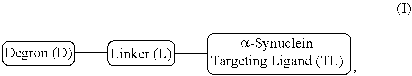

wherein the targeting ligand represents a group that binds α-synuclein protein, the degron represents a moiety that binds an E3 ubiquitin ligase, and the linker represents a moiety that covalently connects the degron and the targeting ligand
wherein

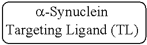

Targeting Ligand (TL) is represented by the formula TL-1, TL-2, TL-3, TL-4, or TL-5:

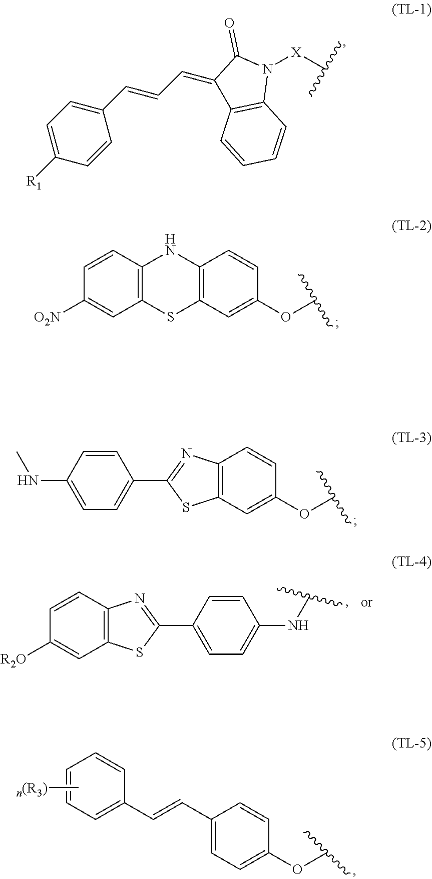

wherein

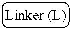

is a polyethylene glycol chain which terminates at either or both termini in —R′C(O)N(R′)R′—, wherein R′ is H or C₁-C₆ alkyl; or
an alkylene chain or a bivalent alkylene chain, either of which may be interrupted by, and/or terminate at either or both termini in —R′C(O)N(R′)R′— or —R′C(O)OR′—, wherein R′ is H or C₁-C₆ alkyl; and
wherein

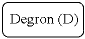

is represented by the formula D1-a to D1-i:

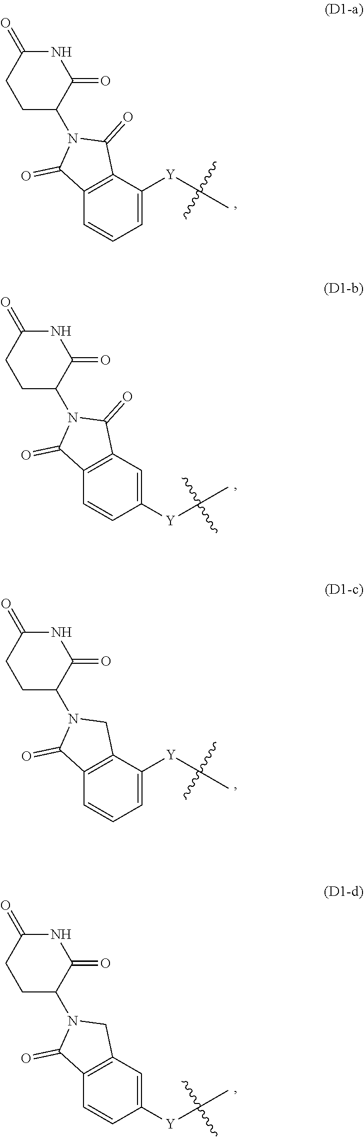

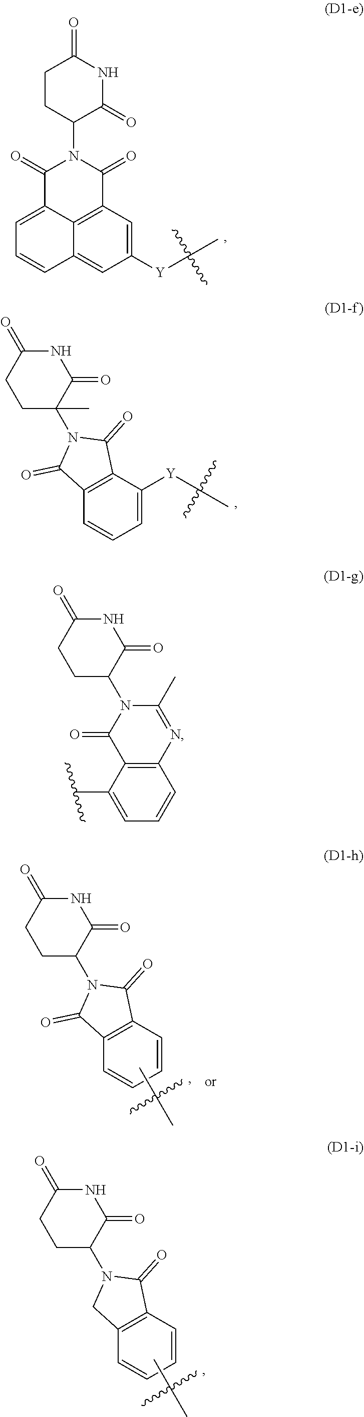

wherein X, Y, R₁, R₂, R₃, and n are as defined herein, or a pharmaceutically acceptable salt or stereoisomer thereof.

Another aspect of the present invention is directed to a pharmaceutical composition containing a therapeutically effective amount of the bispecific compound of formula (I) or a pharmaceutically acceptable salt or stereoisomer thereof, and a pharmaceutically acceptable carrier.

In another aspect of the present invention, methods of making the bispecific compounds are provided.

A further aspect of the present invention is directed to a method of treating a neurodegenerative disease or disorder involving aberrant α-synuclein protein activity, that includes administering a therapeutically effective amount of the bispecific compound of formula (I) or a pharmaceutically acceptable salt or stereoisomer thereof, to a subject in need thereof.

Without intending to be bound by any particular theory of operation, the bispecific compounds of formula (I) (also referred to herein as PROTACs or degraders) are believed to promote the degradation of α-synuclein protein via cells' Ubiquitin/Proteasome System, whose function is to routinely identify and remove damaged proteins. After the destruction of an α-synuclein protein molecule, the degrader is released and continues to be active. Thus, by engaging and exploiting the body's own natural protein disposal system, the bispecific compounds of the present invention may represent a potential improvement over current small molecule inhibitors of α-synuclein. Thus, effective intracellular concentrations of the degraders may be significantly lower than for small molecule α-synuclein inhibitors. Bispecific compounds of the present invention may be more potent inhibitors of α-synuclein protein than known inhibitors.

Bispecific compounds of the present invention may offer at least one additional advantage including improved pharmacodynamics. The use of targeted degradation technology to recruit E3-ligase adaptor proteins to α-synuclein protein aggregates via the bispecific compound leads to ubiquitination and clearance through the proteasome.

### BRIEF DESCRIPTION OF THE DRAWINGS

FIG. 1A-FIG. 1K is a series of graphs showing cellular CRBN target engagement data. FIG. 1A is a graph showing the half maximal inhibitory concentration (IC₅₀) for bispecific compound 1 binding to CRBN. FIG. 1B is a graph showing the IC₅₀ for bispecific compound 2 binding to CRBN. FIG. 1C is a graph showing the IC₅₀ for bispecific compound 7 binding to CRBN. FIG. 1D is a graph showing the IC₅₀ for bispecific compound 8 binding to CRBN. FIG. 1E is a graph showing the IC₅₀ for bispecific compound 9 binding to CRBN. FIG. 1F is a graph showing the IC₅₀ for bispecific compound 10 binding to CRBN. FIG. 1G is a graph showing the IC₅₀ for bispecific compound 11 binding to CRBN. FIG. 1H is a graph showing the IC₅₀ for bispecific compound 12 binding to CRBN. FIG. 1I is a graph showing the ICs for bispecific compound 13 binding to CRBN. FIG. 1J is a graph showing the IC₅₀ for control A binding to CRBN. FIG. 1K is a graph showing the IC₅₀ for control B binding to CRBN.

### DETAILED DESCRIPTION OF THE INVENTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present invention.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2% or 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

With respect to compounds of the present invention, and to the extent the following terms are used herein to further describe them, the following definitions apply.

As used herein, the term “alkyl” refers to a saturated linear or branched-chain monovalent hydrocarbon radical. In one embodiment, the alkyl radical is a C₁-C₁₈ group. In other embodiments, the alkyl radical is a C₀-C₆, C₀-C₅, C₀-C₃, C₁-C₁₂, C₁-C₈, C₁-C₆, C₁-C₅, C₁-C₄ or C₁-C₃ group (wherein C₀ alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, heptyl, octyl, nonyl, decyl, undecyl and dodecyl. In some embodiments, an alkyl group is a C₁-C₃ alkyl group. In some embodiments, an alkyl group is a C₁-C₂ alkyl group, or a methyl group.

As used herein, the term “alkylene” refers to a straight or branched divalent hydrocarbon chain linking the rest of the molecule to a radical group, consisting solely of carbon and hydrogen, containing no unsaturation and having from one to 12 carbon atoms, for example, methylene, ethylene, propylene, n-butylene, and the like. The alkylene chain may be attached to the rest of the molecule through a single bond and to the radical group through a single bond. In some embodiments, the alkylene group contains one to 8 carbon atoms (C₁-C₈ alkylene). In other embodiments, an alkylene group contains one to 5 carbon atoms (C₁-C₅ alkylene). In other embodiments, an alkylene group contains one to 4 carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “alkenyl” refers to a linear or branched-chain monovalent hydrocarbon radical with at least one carbon-carbon double bond. An alkenyl includes radicals having “cis” and “trans” orientations, or alternatively, “E” and “Z” orientations. In one example, the alkenyl radical is a C₂-C₁₈ group. In other embodiments, the alkenyl radical is a C₂-C₁₂, C₂-C₁₀, C₂-C₈, C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

As used herein, the term “alkynyl” refers to a linear or branched monovalent hydrocarbon radical with at least one carbon-carbon triple bond. In one example, the alkynyl radical is a C₂-C₁₈ group. In other examples, the alkynyl radical is C₂-C₁₂, C₂-C₁₀, C₂-C₈, C₂-C₆ or C₂-C₃. Examples include ethynyl prop-1-ynyl, prop-2-ynyl, but-1-ynyl, but-2-ynyl and but-3-ynyl.

The terms “alkoxyl” or “alkoxy” as used herein refer to an alkyl group, as defined above, having an oxygen radical attached thereto, and which is the point of attachment. Representative alkoxyl groups include methoxy, ethoxy, propyloxy, tert-butoxy and the like. An “ether” is two hydrocarbyl groups covalently linked by an oxygen. Accordingly, the substituent of an alkyl that renders that alkyl an ether is or resembles an alkoxyl, such as can be represented by one of —O-alkyl, —O-alkenyl, and —O-alkynyl.

As used herein, the term “halogen” (or “halo” or “halide”) refers to fluorine, chlorine, bromine, or iodine.

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Thus, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). The term carbocyclyl includes mono-, bi-, tri-, fused, bridged, and spiro-ring systems, and combinations thereof. In one embodiment, carbocyclyl includes 3 to 15 carbon atoms (C₃-C₁₅). In one embodiment, carbocyclyl includes 3 to 12 carbon atoms (C₃-C₁₂). In another embodiment, carbocyclyl includes C₃-C₈, C₃-C₁₀ or C₅-C₁₀. In another embodiment, carbocyclyl, as a monocycle, includes C₃-C₈, C₃-C₆ or C₅-C₆. In some embodiments, carbocyclyl, as a bicycle, includes C₇-C₁₂. In another embodiment, carbocyclyl, as a spiro system, includes C₅-C₁₂. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Thus, the term carbocyclic also embraces carbocyclylalkyl groups which as used herein refer to a group of the formula —R^{c}-carbocyclyl where R^{c} is an alkylene chain. The term carbocyclic also embraces carbocyclylalkoxy groups which as used herein refer to a group bonded through an oxygen atom of the formula —O—R^{c}-carbocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, aryl includes groups having 6-18 carbon atoms. In another embodiment, aryl includes groups having 6-10 carbon atoms. Examples of aryl groups include phenyl, naphthyl, anthracyl, biphenyl, phenanthrenyl, naphthacenyl, 1,2,3,4-tetrahydronaphthalenyl, 1H-indenyl, 2,3-dihydro-1H-indenyl, naphthyridinyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring.

Thus, the term aryl embraces aralkyl groups (e.g., benzyl) which as disclosed above refer to a group of the formula —R^{c}-aryl where R^{c} is an alkylene chain such as methylene or ethylene. In some embodiments, the aralkyl group is an optionally substituted benzyl group. The term aryl also embraces aralkoxy groups which as used herein refer to a group bonded through an oxygen atom of the formula —O—R^{c}-aryl where R^{c} is an alkylene chain such as methylene or ethylene.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., O, N, N(O), S, S(O), or S(O)₂). The term heterocyclyl includes mono-, bi-, tri-, fused, bridged, and spiro-ring systems, and combinations thereof. In some embodiments, a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. In some embodiments, a heterocyclyl refers to a heteroaryl ring system, such as a 5 to 14 membered heteroaryl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

In some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from nitrogen, sulfur or oxygen. In some embodiments, heterocyclyl includes 4- to 6-membered monocycles having one or more heteroatoms selected from nitrogen, sulfur or oxygen. In some embodiments, heterocyclyl includes 3-membered monocycles. In some embodiments, heterocyclyl includes 4-membered monocycles. In some embodiments, heterocyclyl includes 5-6 membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocyclyls containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Example 6-membered heterocyclyls contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are yet other examples of heterocyclyl groups. In some embodiments, a heterocyclic group includes a heterocyclic ring fused to one or more (e.g., 1, 2 or 3) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Thus, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R^{c}— heterocyclyl where R^{c} is an alkylene chain. The term heterocyclic also embraces heterocyclylalkoxy groups which as used herein refer to a radical bonded through an oxygen atom of the formula —O—R^{c}-heterocyclyl where R^{c} is an alkylene chain.

As used herein, the term “heteroaryl” used alone or as part of a larger moiety (e.g., “heteroarylalkyl” (also “heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. In one embodiment, heteroaryl includes 5-6 membered monocyclic aromatic groups where one or more ring atoms is nitrogen, sulfur or oxygen. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring.

Thus, the term heteroaryl embraces N-heteroaryl groups which as used herein refer to a heteroaryl group as defined above containing at least one nitrogen and where the point of attachment of the heteroaryl group to the rest of the molecule is through a nitrogen atom in the heteroaryl group. The term heteroaryl also embraces C-heteroaryl groups which as used herein refer to a heteroaryl group as defined above and where the point of attachment of the heteroaryl group to the rest of the molecule is through a carbon atom in the heteroaryl group. The term heteroaryl also embraces heteroarylalkyl groups which as disclosed above refer to a group of the formula —R^{c}-heteroaryl, wherein R^{c} is an alkylene chain as defined above. The term heteroaryl also embraces heteroaralkoxy (or heteroarylalkoxy) groups which as used herein refer to a group bonded through an oxygen atom of the formula —O—R^{c}-heteroaryl, where R^{c} is an alkylene group as defined above.

Any of the groups described herein may be substituted or unsubstituted. As used herein, the term “substituted” broadly refers to all permissible substituents with the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc. Representative substituents include halogens, hydroxyl groups, and any other organic groupings containing any number of carbon atoms, e.g., 1-14 carbon atoms, and which may include one or more (e.g., 1, 2, 3, or 4) heteroatoms such as oxygen, sulfur, and nitrogen grouped in a linear, branched, or cyclic structural format.

To the extent not disclosed otherwise for any particular group(s), representative examples of substituents may include alkyl, substituted alkyl (e.g., C₁-C₆, C₁-C₅, C₁-C₄, C₁-C₃, C₁-C₂, C₁), alkoxy (e.g., C₁-C₆, C₁-C₅, C₁-C₄, C₁-C₃, C₁-C₂, C₁), substituted alkoxy (e.g., C₁-C₆, C₁-C₅, C₁-C₄, C₁-C₃, C₁-C₂, C₁), haloalkyl (e.g., CF₃), alkenyl (e.g., C₂-C₆, C₂-C₅, C₂-C₄, C₂-C₃, C₂), substituted alkenyl (e.g., C₂-C₆, C₂-C₅, C₂-C₄, C₂-C₃, C₂), alkynyl (e.g., C₂-C₆, C₂-C₅, C₂-C₄, C₂-C₃, C₂), substituted alkynyl (e.g., C₂-C₆, C₂-C₅, C₂-C₄, C₂-C₃, C₂), cyclic (e.g., C₃-C₁₂, C₅-C₆), substituted cyclic (e.g., C₃-C₁₂, C₅-C₆), carbocyclic (e.g., C₃-C₁₂, C₅-C₆), substituted carbocyclic (e.g., C₃-C₁₂, C₅-C₆), heterocyclic (e.g., C₃-C₁₂, C₅-C₆), substituted heterocyclic (e.g., C₃-C₁₂, C₅-C₆), aryl (e.g., benzyl and phenyl), substituted aryl (e.g., substituted benzyl or phenyl), heteroaryl (e.g., pyridyl or pyrimidyl), substituted heteroaryl (e.g., substituted pyridyl or pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., C₆-C₁₂, C₆), alkylthio (e.g., C₁-C₆), substituted alkylthio (e.g., C₁-C₆), arylthio (e.g., C₆-C₁₂, C₆), substituted arylthio (e.g., C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

The term “binding” as it relates to interaction between the targeting ligand and the targeted protein, which in this case is α-synuclein protein and mutant or misfolded forms thereof, refers to an inter-molecular interaction that is substantially specific in that binding of the targeting ligand with other proteinaceous entities present in the cell may be functionally insignificant. The present bispecific compounds bind and recruit α-synuclein protein for selective degradation.

The term “binding” as it relates to interaction between the degron and the E3 ubiquitin ligase, typically refers to an inter-molecular interaction that may or may not exhibit an affinity level that equals or exceeds that affinity between the targeting ligand and the target protein, but nonetheless wherein the affinity is sufficient to achieve recruitment of the ligase to the targeted degradation and the selective degradation of the targeted protein.

Broadly, the bispecific compounds of the present invention have a structure represented by formula (I):

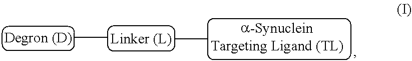

wherein the targeting ligand represents a group that binds α-synuclein protein, the degron represents a moiety that binds an E3 ubiquitin ligase, and the linker represents a moiety that covalently connects the degron and the targeting ligand.

### Targeting Ligands

The α-synuclein targeting ligand has a structure represented by any one of formulas TL-1, TL-2, TL-3, TL-4 and TL-5:

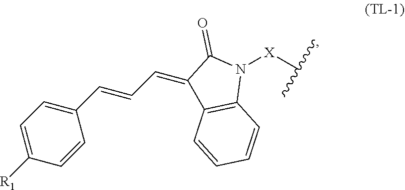

wherein:
X is absent or

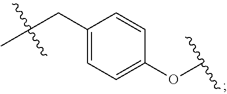 [SMILES: *Cc1ccc(O*)cc1]

and
R₁ is nitro or amino;

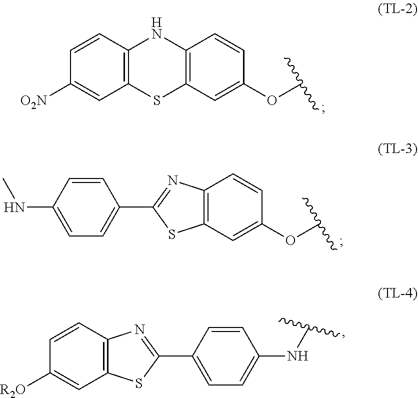

wherein:
R₂ is hydrogen or methyl; or

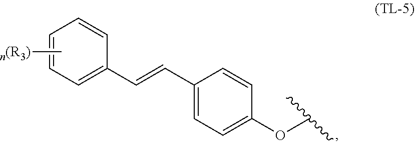

wherein:
R₃ is alkyl, alkenyl, alkynyl, halo, haloalkyl, cycloalkyl, heterocycloalkyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylaminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, azido, phosphinyl, phosphoryl, aryl, or heteroaryl, said R₃ groups may be further optionally substituted; and n is 0, 1, 2, 3, 4, or 5.

Thus, in some embodiments, the bispecific compounds of the present invention may have a structure as represented by any one of formulas I-1, I-2, I-3, I-4, and I-5:

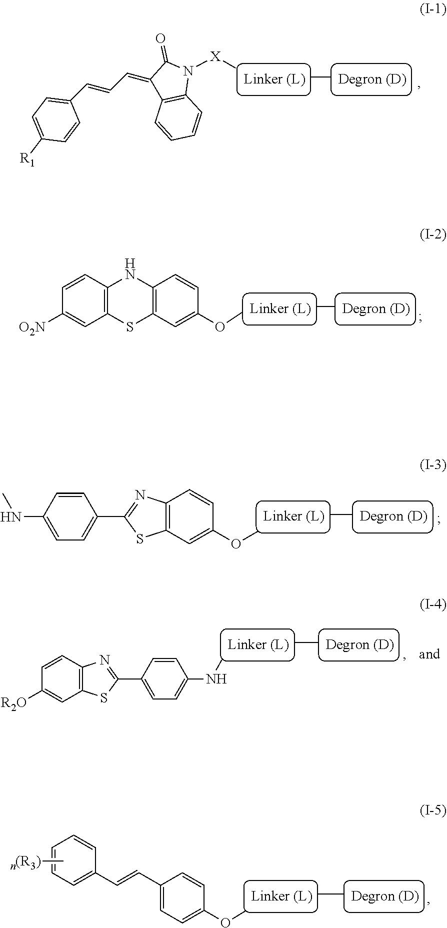

or a pharmaceutically acceptable salt or stereoisomer thereof.

In some embodiments, R₁ is NH₂ and X is absent and the bispecific compound is represented by the formula I-la:

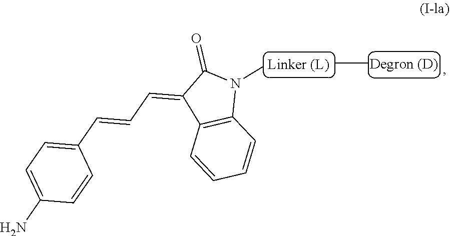

or a pharmaceutically acceptable salt or stereoisomer thereof.

In some embodiments, R₁ is NO₂ and X is

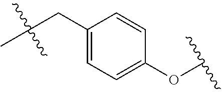 [SMILES: *Cc1ccc(O*)cc1]

and the bispecific compound is represented by the formula I-1b:

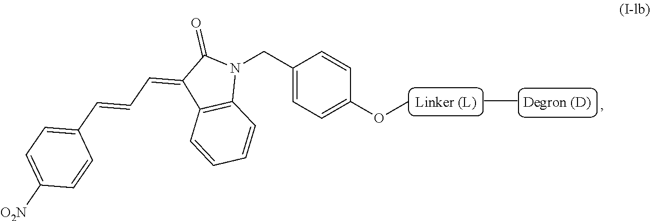

or a pharmaceutically acceptable salt or stereoisomer thereof.

In some embodiments, R₂ is hydrogen and the bispecific compound is represented by the formula (I-4a):

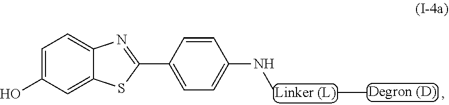

or a pharmaceutically acceptable salt or stereoisomer thereof.

In some embodiments, R₃ is

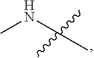 [SMILES: *NC]

and n is 1 and the bispecific compound is represented by the formula (I-5a):

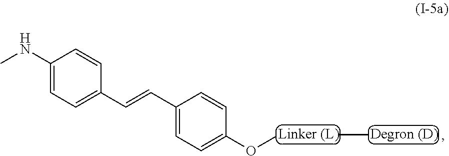

or a pharmaceutically acceptable salt or stereoisomer thereof.

### Linker

In some embodiments, the linker is an alkylene chain or a bivalent alkylene chain, either of which may be interrupted by, and/or terminate at either or both termini in —R′C(O)N(R′)R′—, wherein R′ is H or C₁-C₆ alkyl.

In some embodiments, the linker includes an alkylene chain having 1-10 alkylene units and interrupted by or terminating in

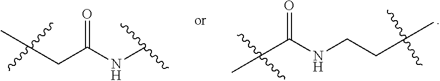 [SMILES: *CC(=O)N* ; *CCNC(*)=O]

Representative examples of alkylene linkers that may be suitable for use in the present invention include the following:

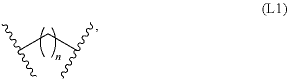

wherein n is an integer of 1-12 (“of” meaning inclusive), e.g., 1-12, 1-11, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10 and 1, 2, 3, 4, 5, 6, 7, 8, 9 and 10, examples of which include:

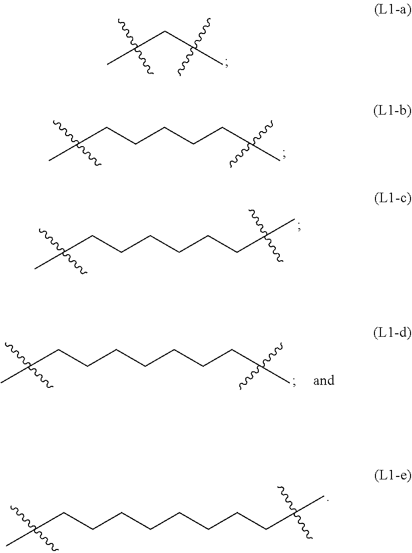

In some embodiments, the linker includes a polyethylene glycol chain which terminates at either or both termini in —R′C(O)N(R′)R′—, wherein R′ is H or C₁-C₆ alkyl.

In some embodiments, the linker comprises a polyethylene glycol chain having 2-8 PEG units and terminating in

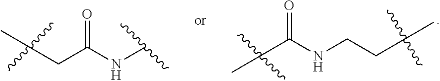 [SMILES: *CC(=O)N* ; *CCNC(*)=O]

In some embodiments, the bispecific compound of formula (I) includes a linker that is represented by any one of the following structures:

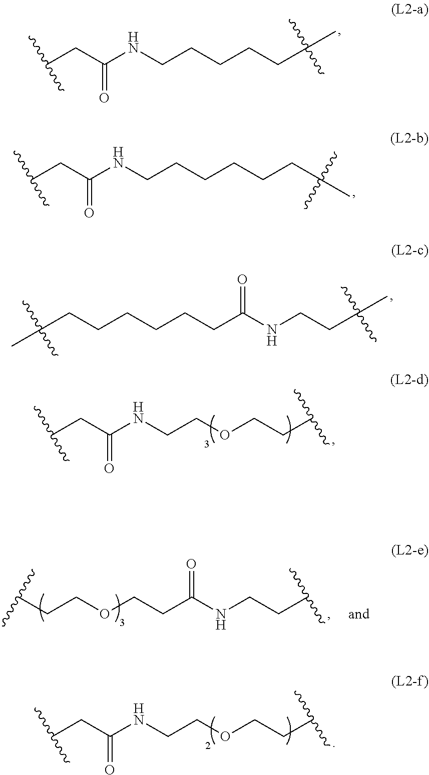

Thus, in some embodiments, the bispecific compounds of the present invention may be represented by any of the following structures:

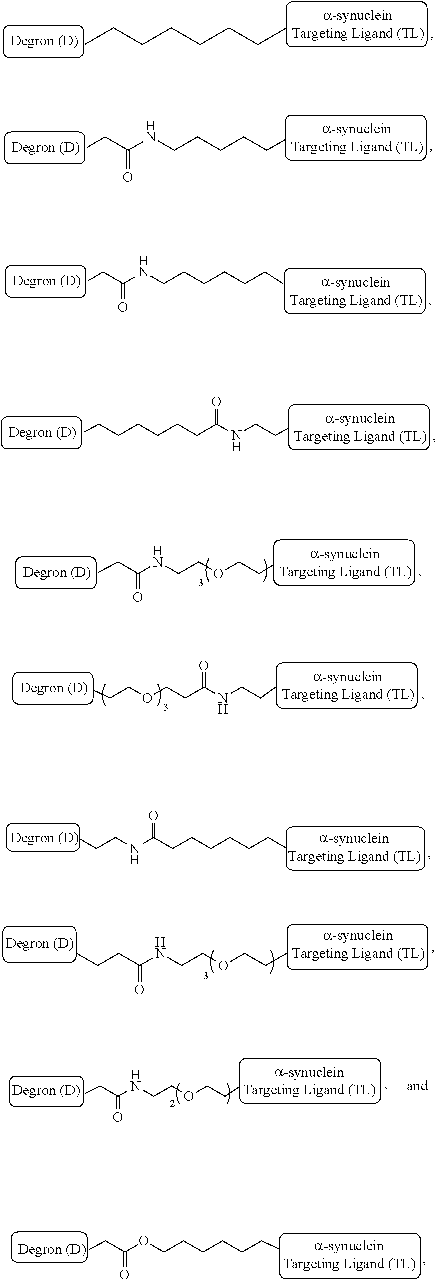

or a pharmaceutically acceptable salt or stereoisomer thereof.

In some embodiments, the bispecific compound of the present invention is represented by any of the following structures:

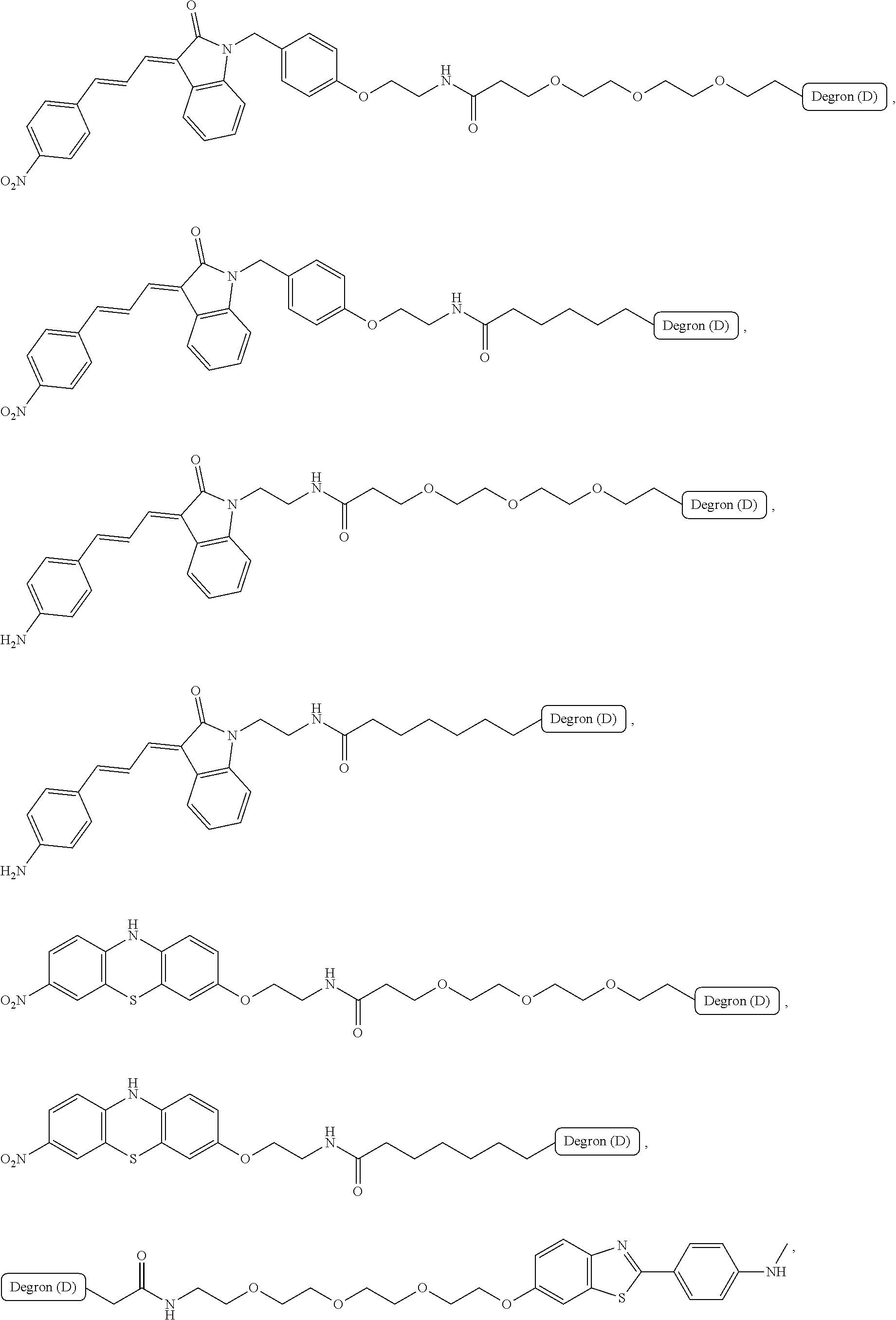

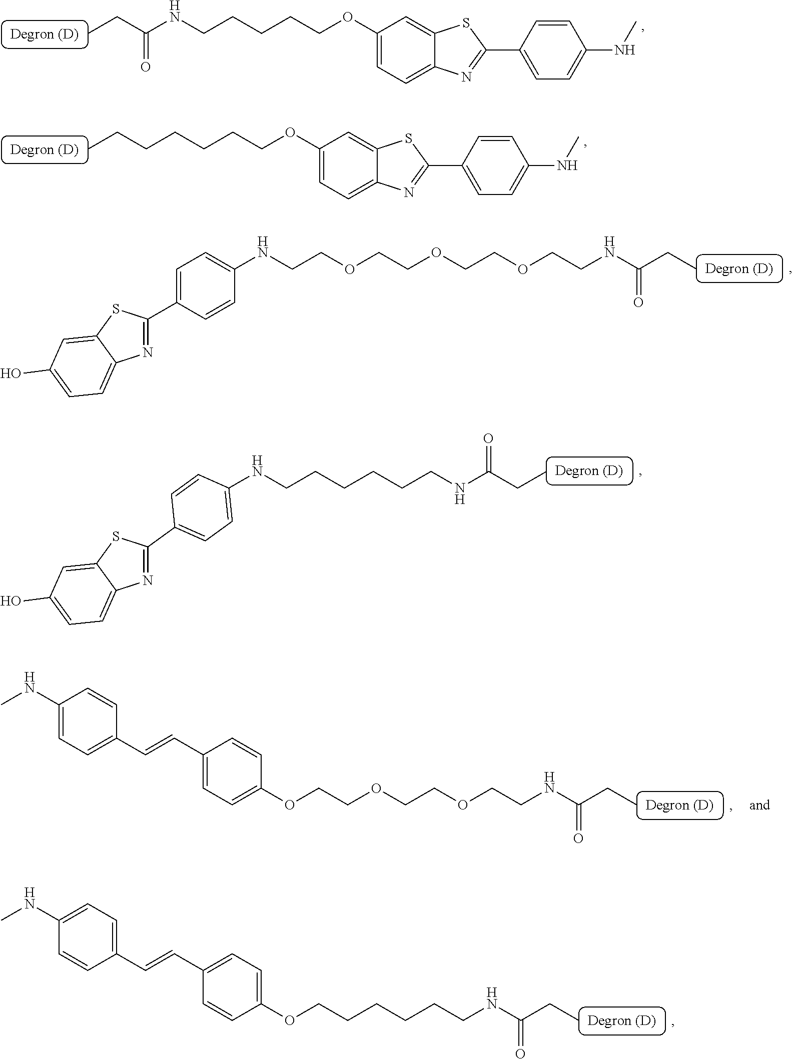

or a pharmaceutically acceptable salt or stereoisomer thereof.

### Degrons

The Ubiquitin-Proteasome Pathway (UPP) is a critical cellular pathway that regulates key regulator proteins and degrades misfolded or abnormal proteins. UPP is central to multiple cellular processes. The covalent attachment of ubiquitin to specific protein substrates is achieved through the action of E3 ubiquitin ligases. These ligases include over 500 different proteins and are categorized into multiple classes defined by the structural element of their E3 functional activity.

The degron binds the E3 ubiquitin ligase which is cereblon (CRBN), and is represented by any one of the following structures:

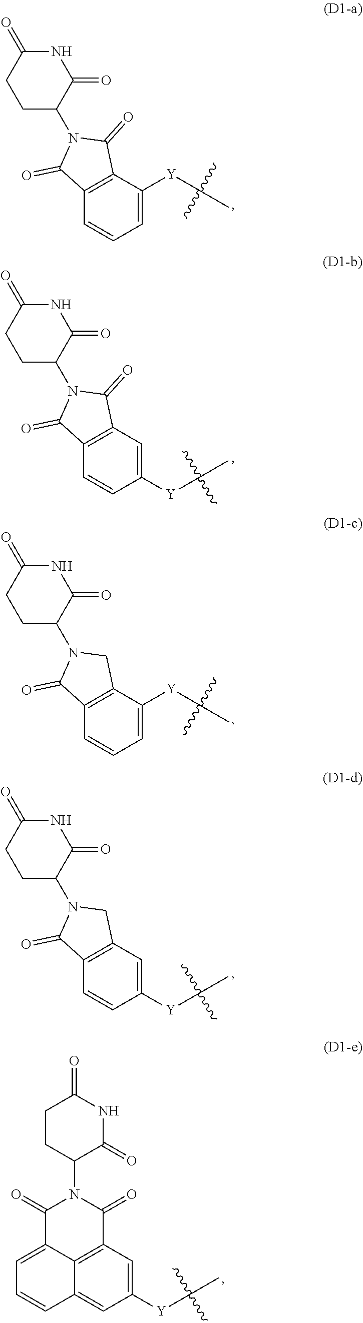

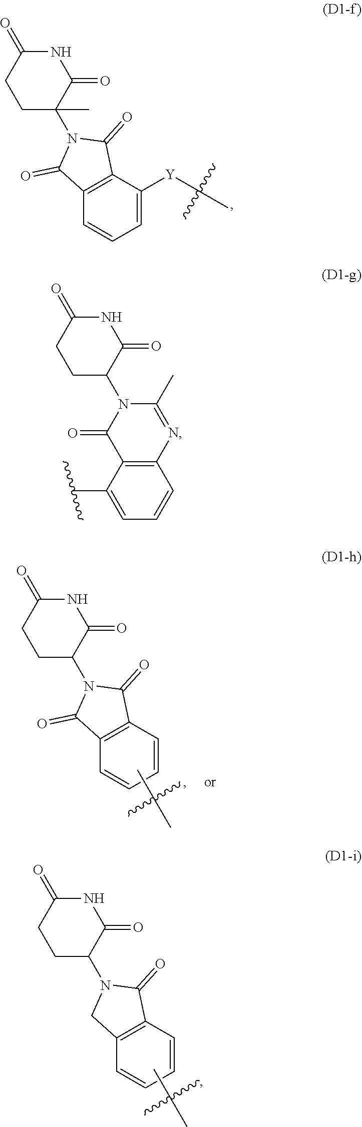

wherein

### Y is NH or O.

Thus, in some embodiments, the bispecific compounds of the present invention may be represented by any of the following structures:

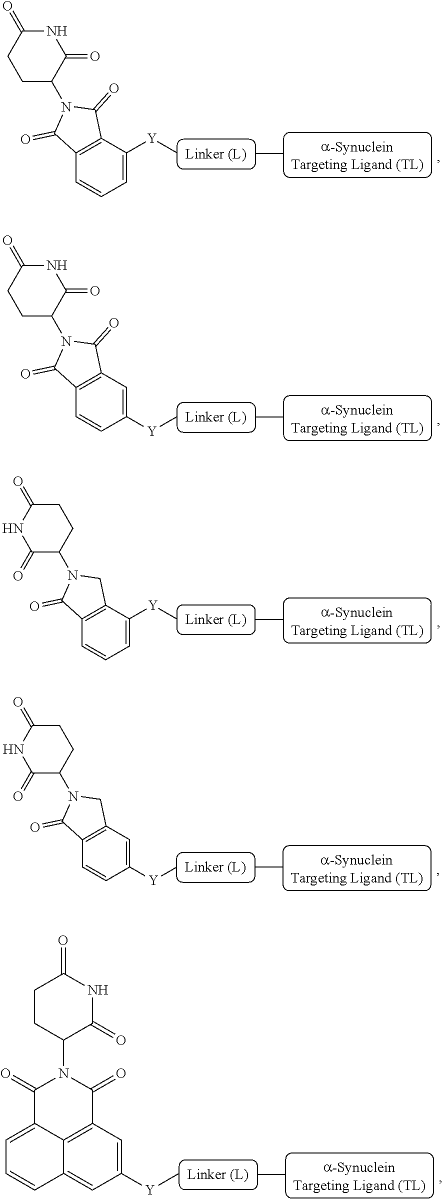

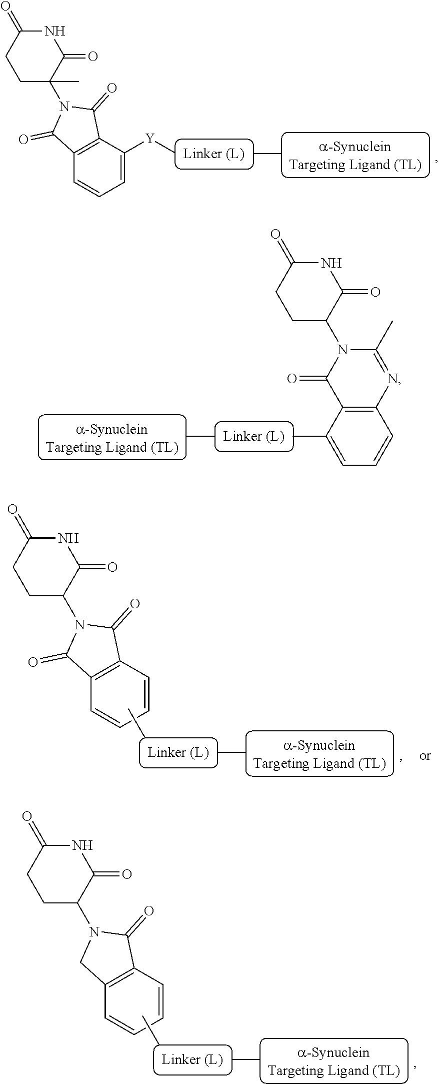

wherein

### Y is NH or O,

or a pharmaceutically acceptable salt or stereoisomer thereof.

In some embodiments, the degron is

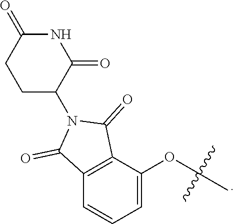 [SMILES: *Oc1cccc2c1C(=O)N(C1CCC(=O)NC1=O)C2=O]

Thus, in some embodiments, the bispecific compounds of the present invention may be represented by

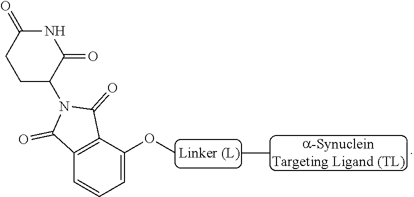

In some embodiments, the bispecific compounds of the present invention are represented by any one of the following structures:

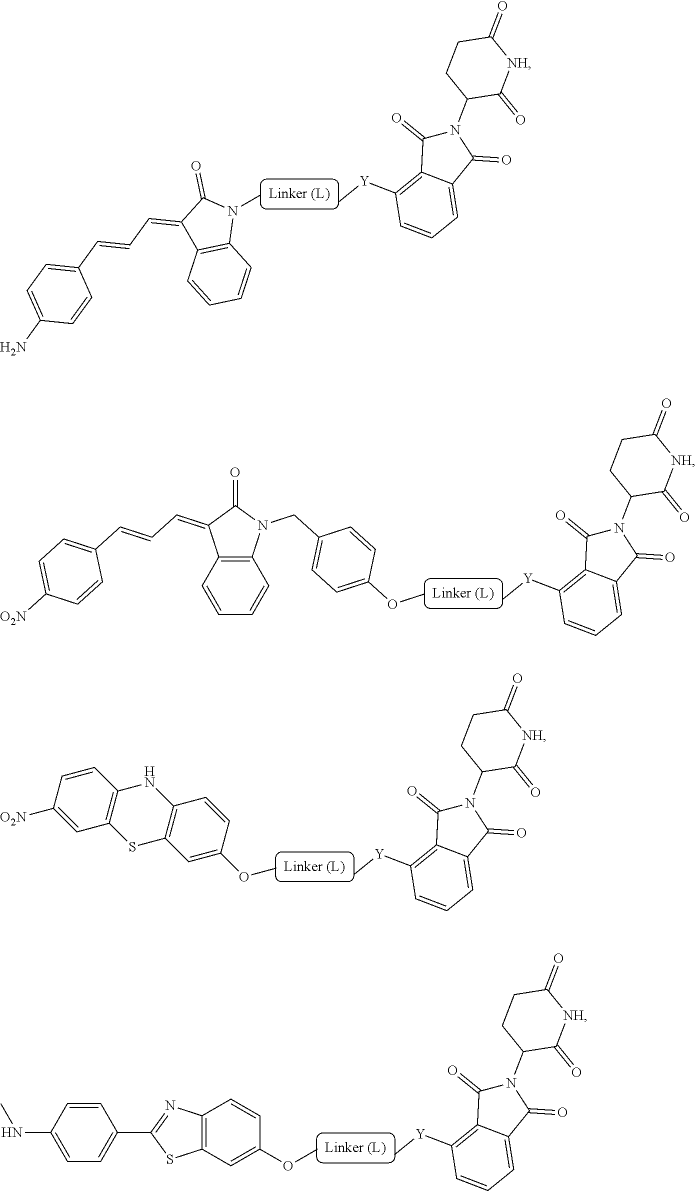

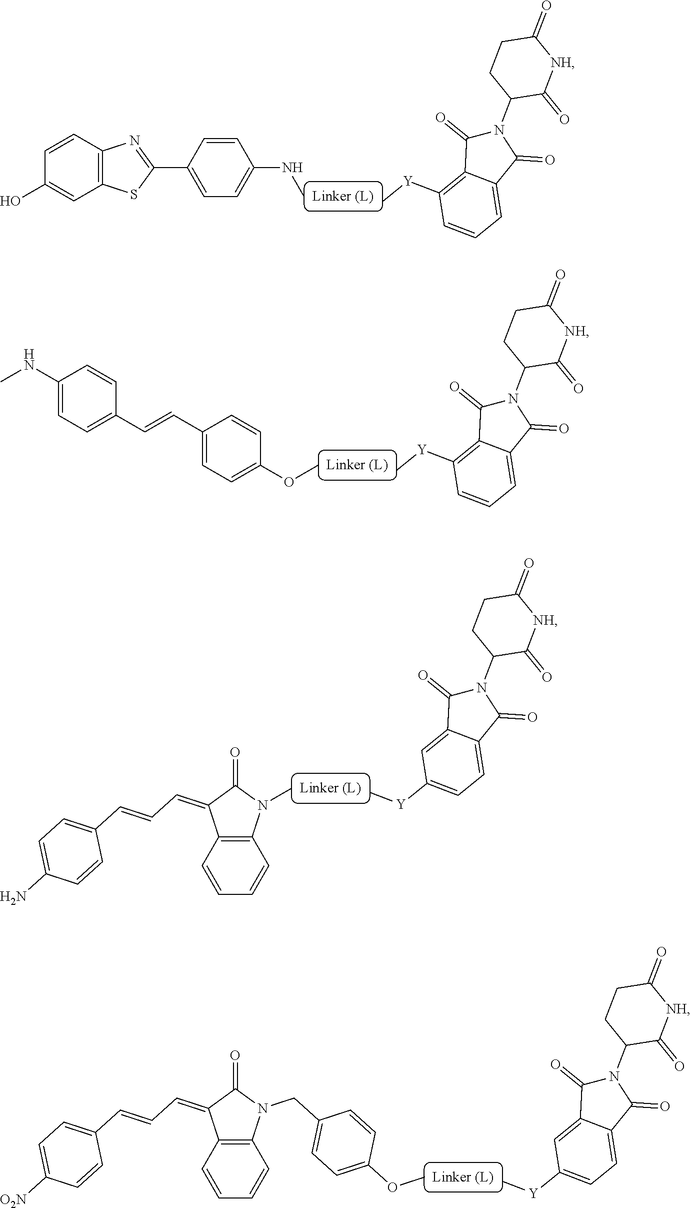

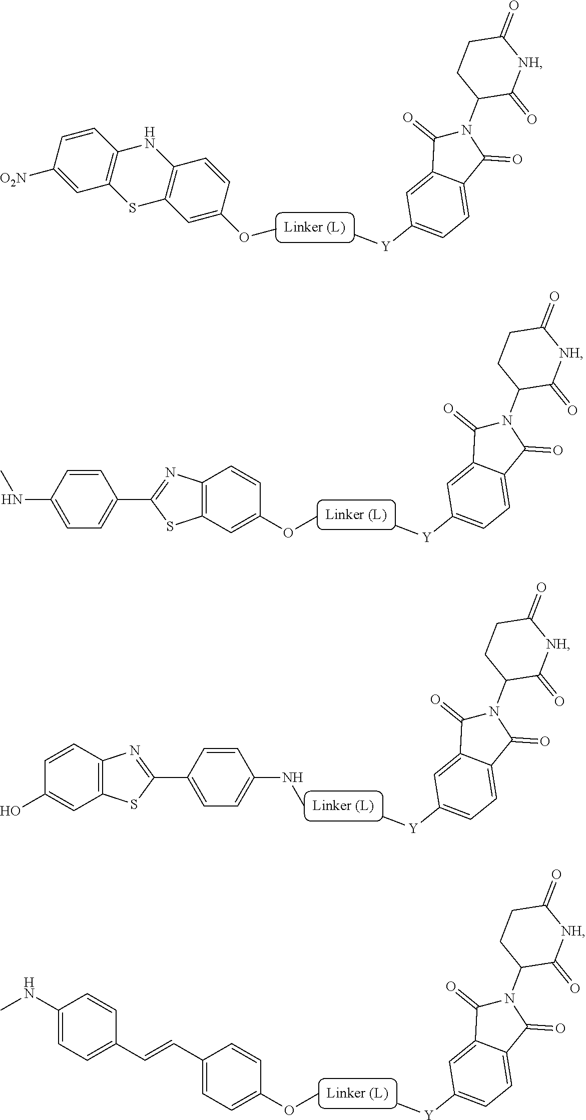

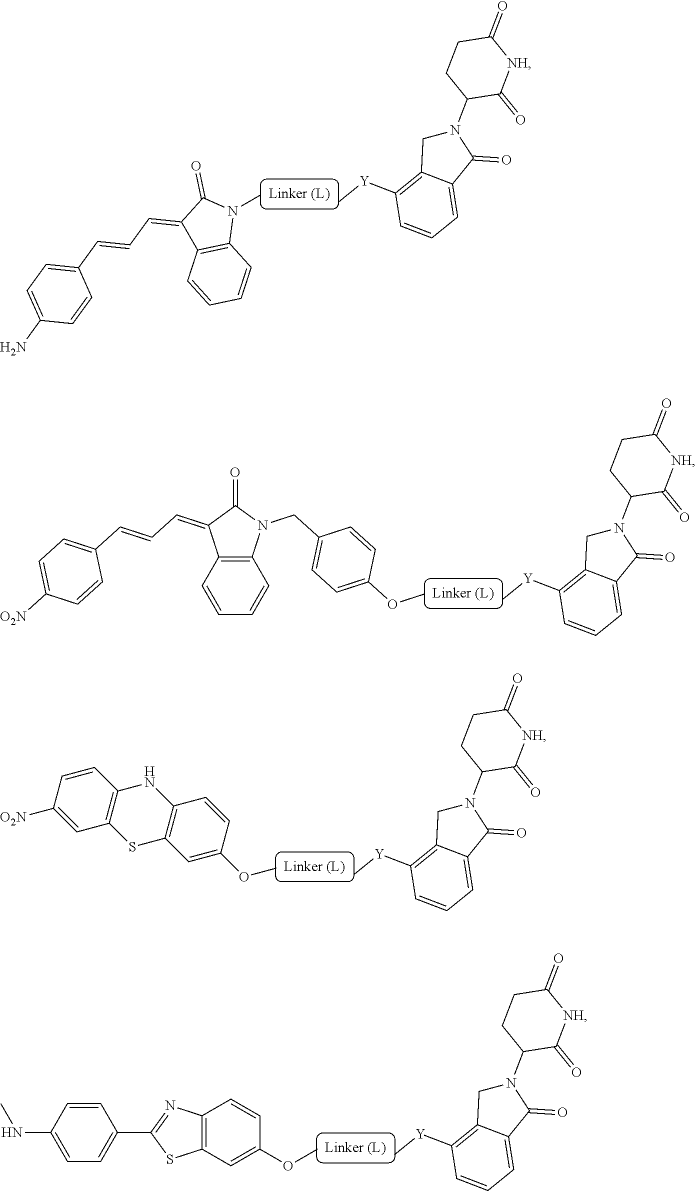

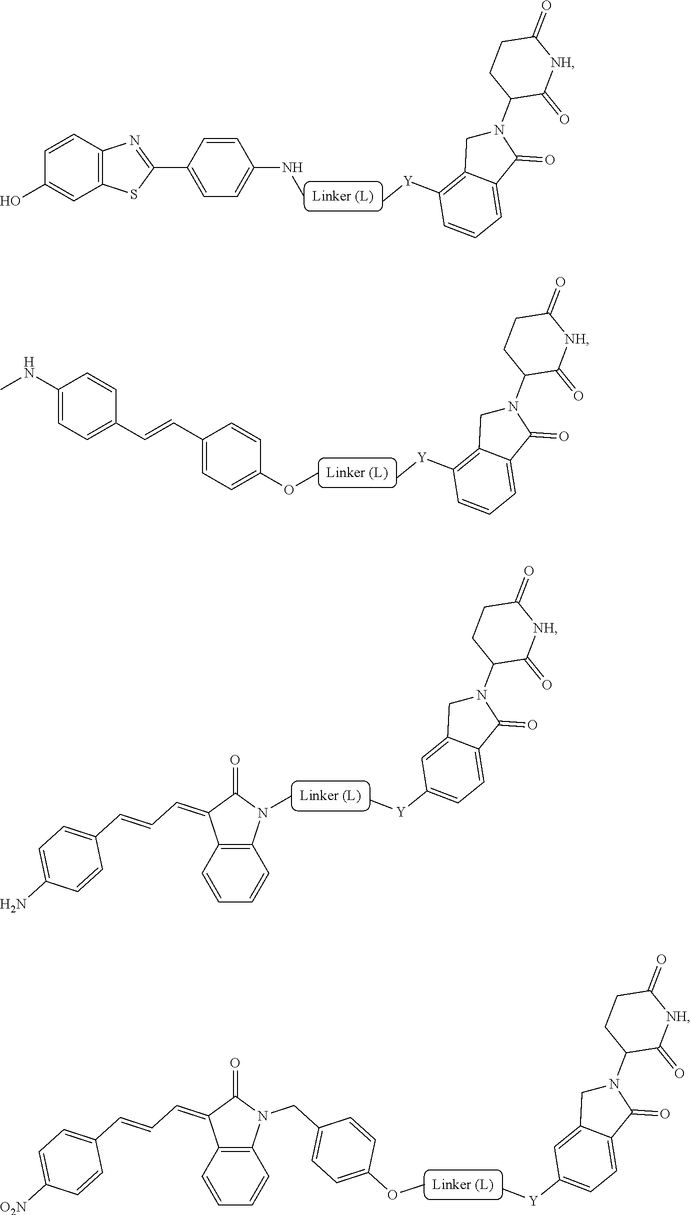

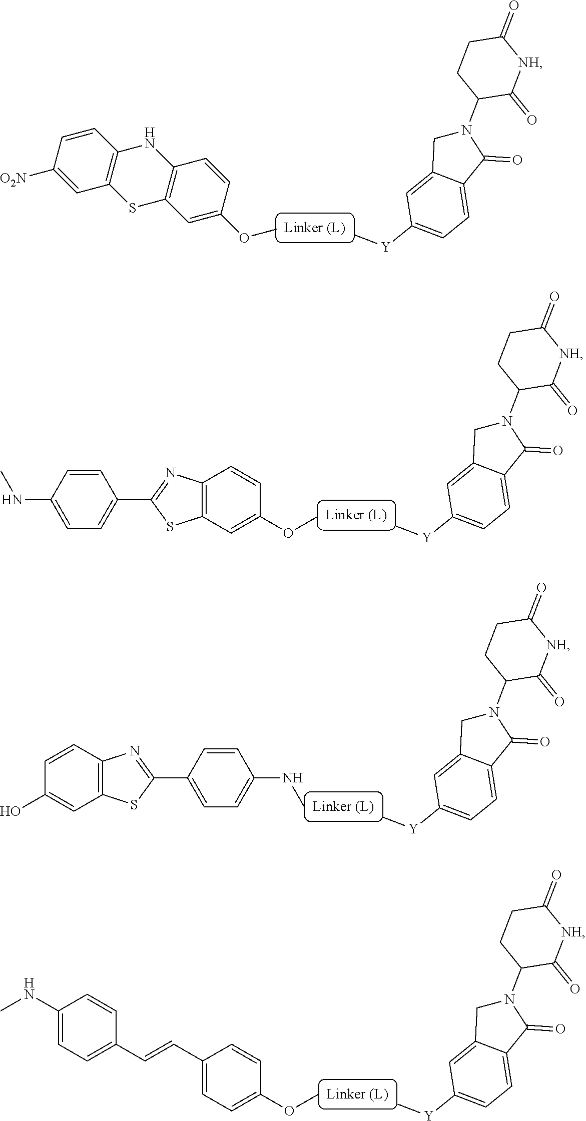

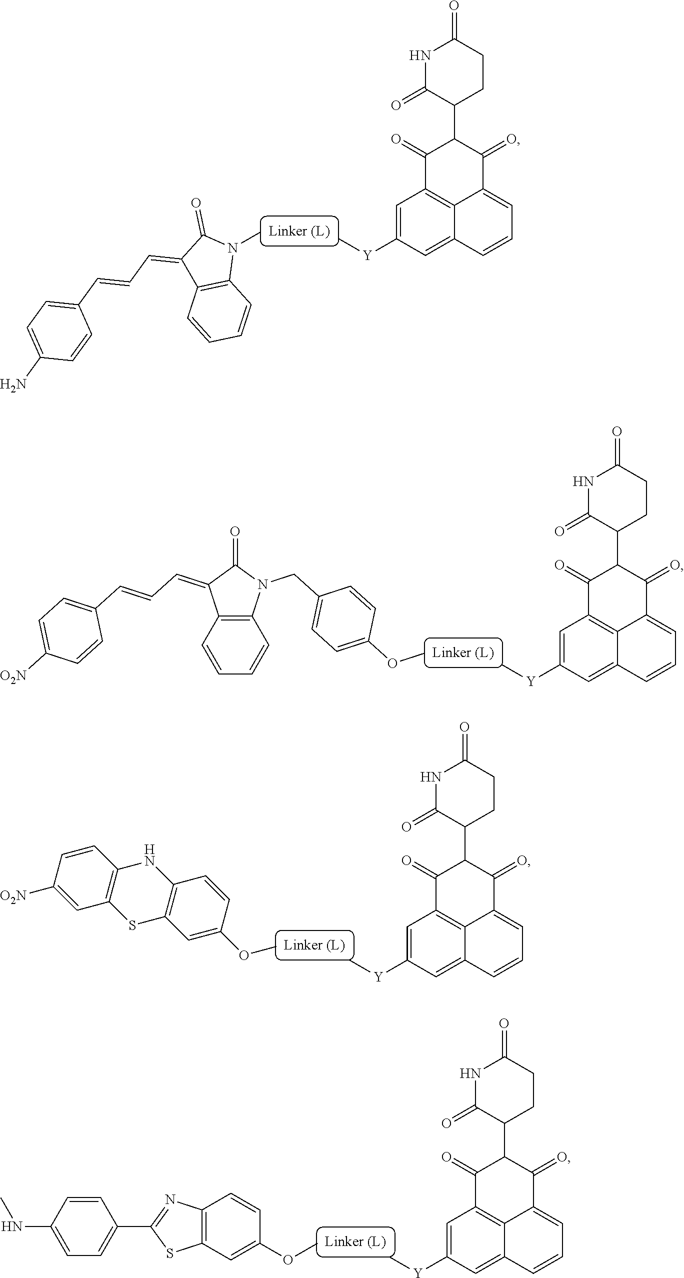

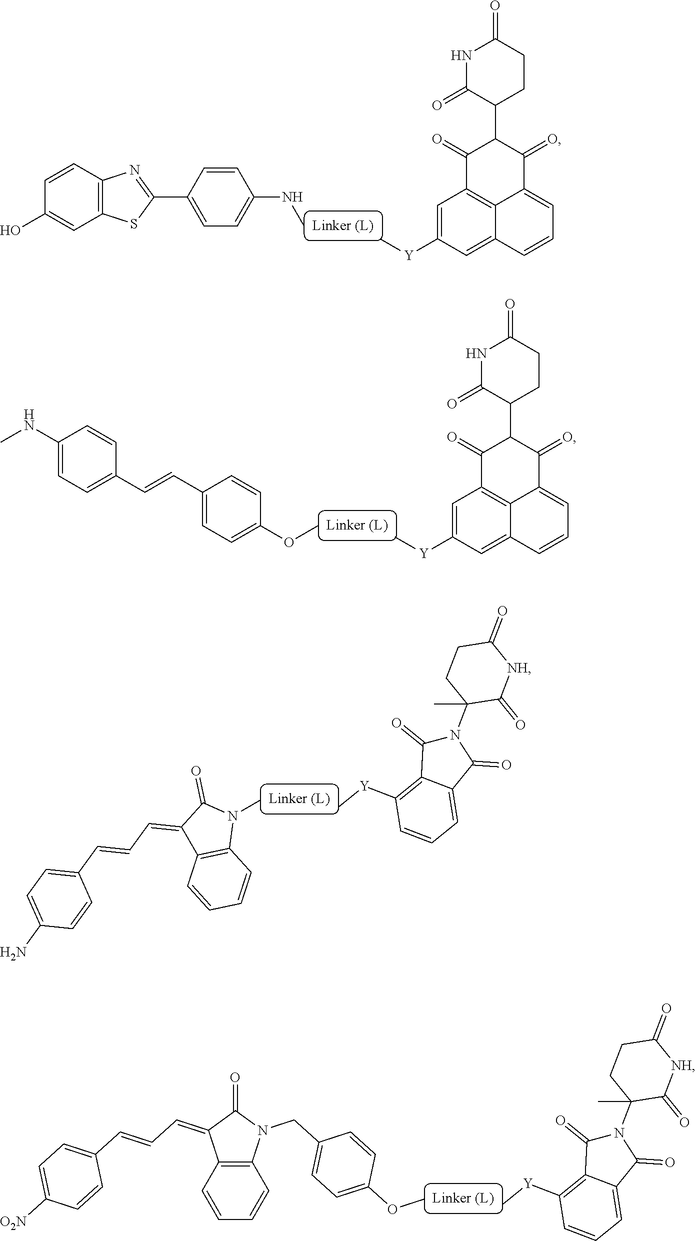

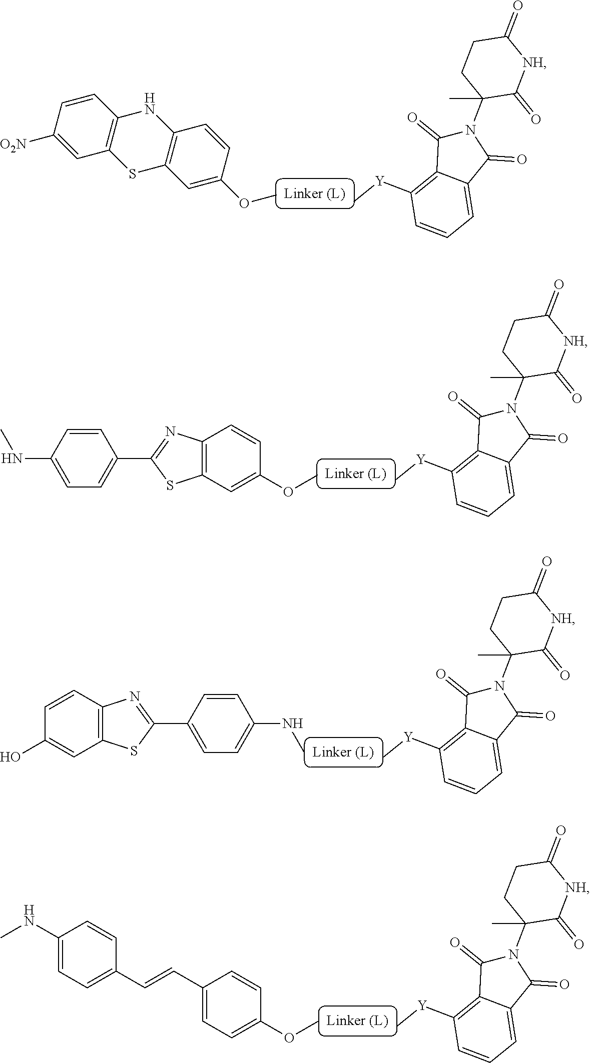

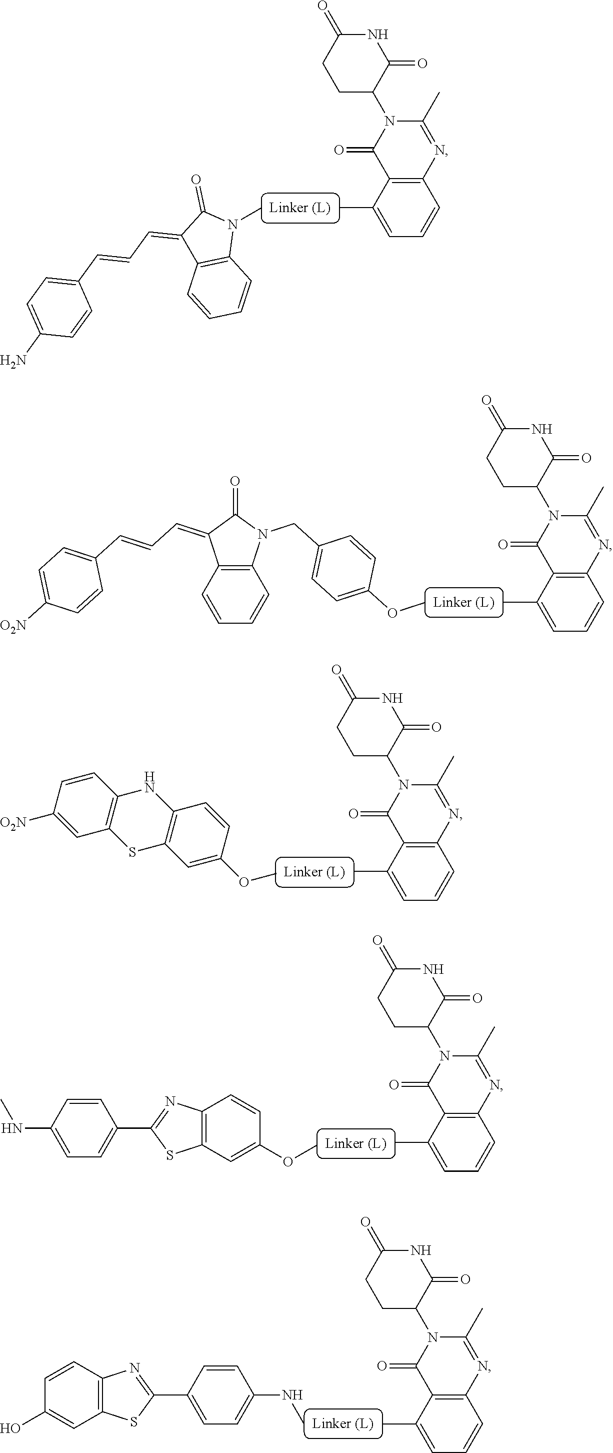

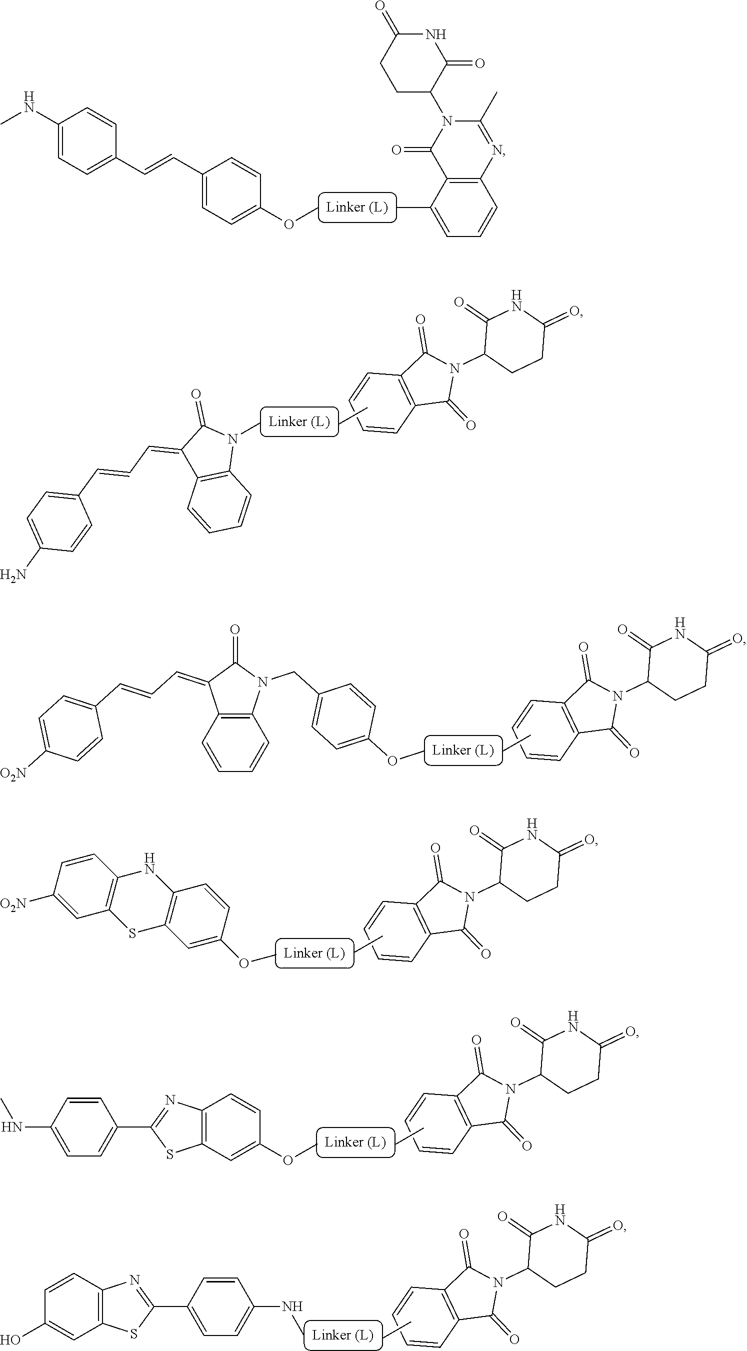

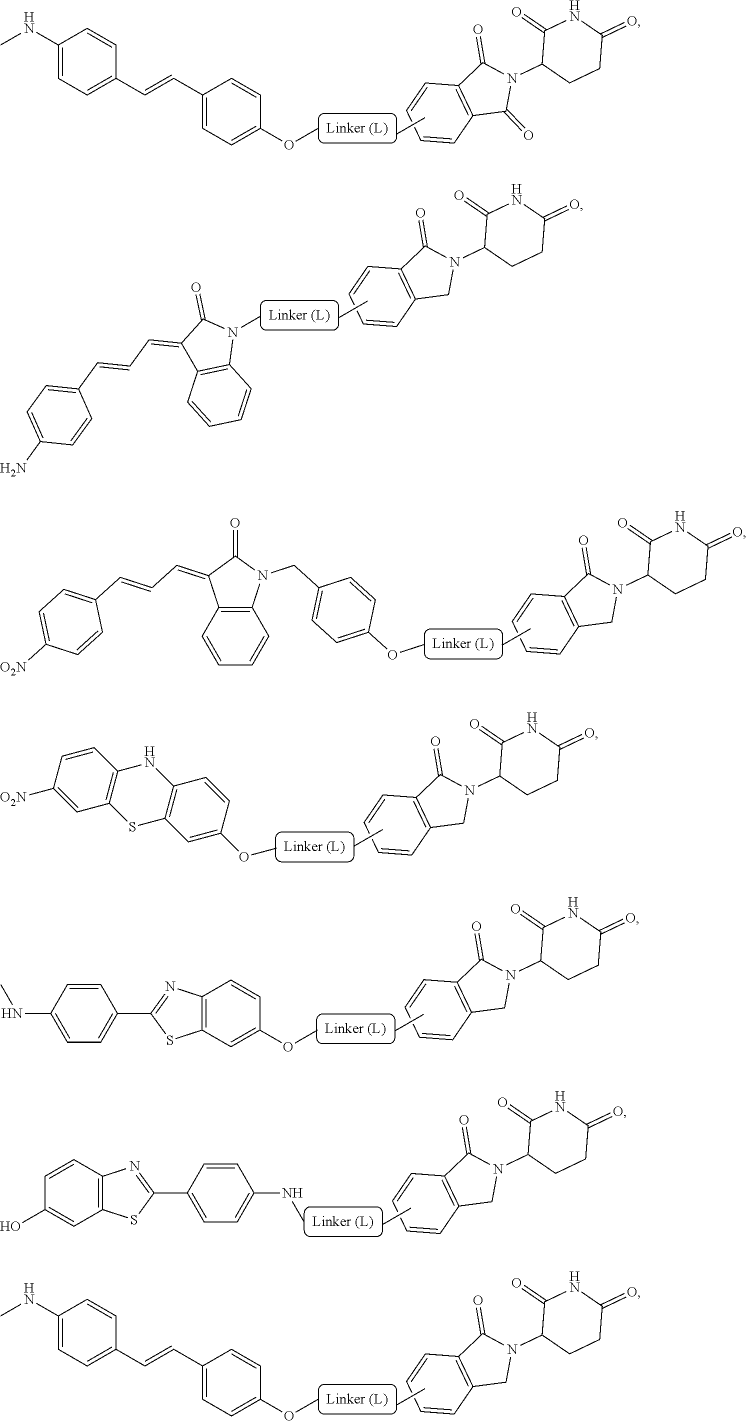

or a pharmaceutically acceptable salt or stereoisomer thereof.

Thus, in some embodiments, the bispecific compounds of this invention are represented by any structures generated by the combination of structures TL1 to TL5, L1 to L2, and D1-a to D1-i, or a pharmaceutically acceptable salt or stereoisomer thereof.

In some embodiments, the bispecific compounds of the present invention are represented by any of the following structures:

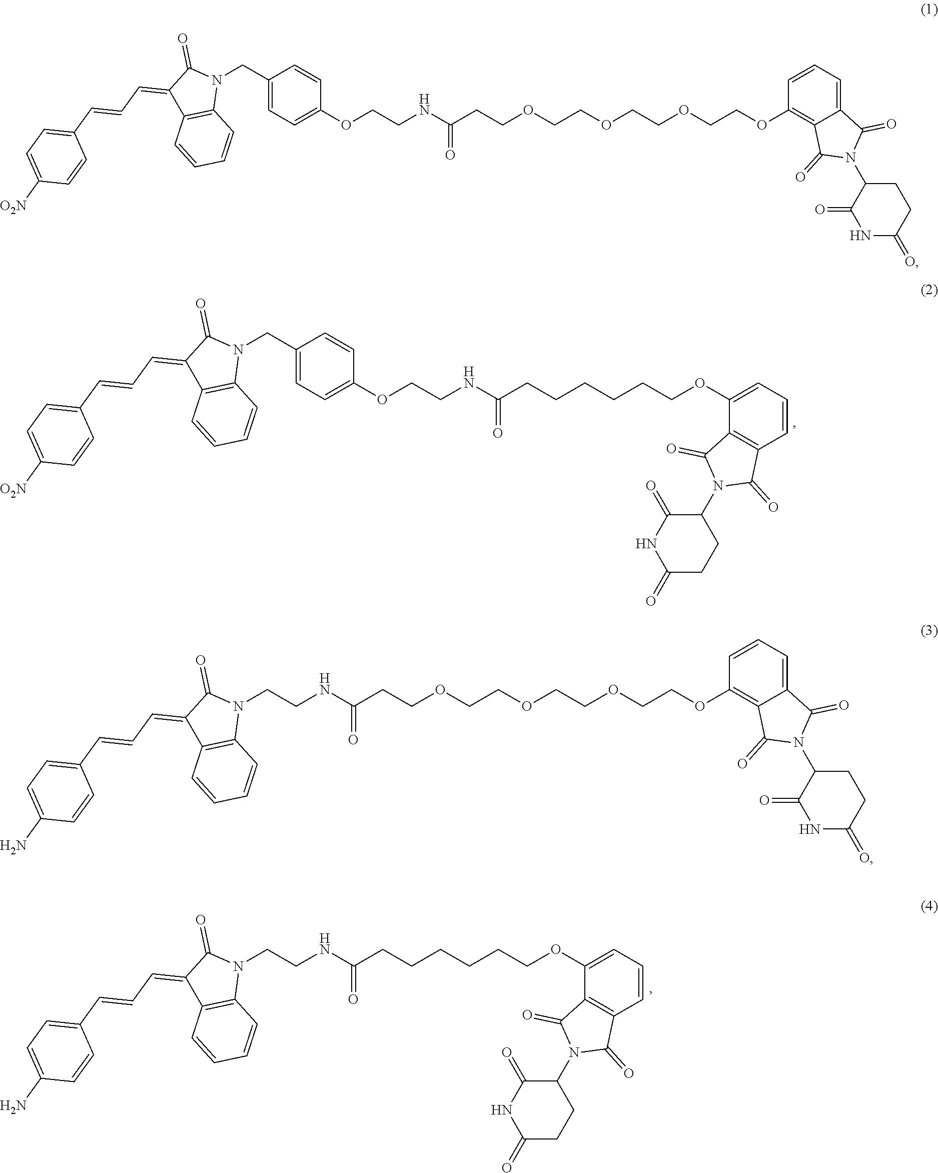 [SMILES: O=C(CCOCCOCCOCCOc1cccc2c1C(=O)N(C1CCC(=O)NC1=O)C2=O)NCCOc1ccc(CN2C(=O)/C(=C/C=C/c3ccc([N+](=O)[O-])cc3)c3ccccc32)cc1 ; O=C(CCCCCCOc1cccc2c1C(=O)N(C1CCC(=O)NC1=O)C2=O)NCCOc1ccc(CN2C(=O)/C(=C/C=C/c3ccc([N+](=O)[O-])cc3)c3ccccc32)cc1 ; Nc1ccc(/C=C/C=C2/C(=O)N(CCNC(=O)CCOCCOCCOCCOc3cccc4c3C(=O)N(C3CCC(=O)NC3=O)C4=O)c3ccccc32)cc1 ; Nc1ccc(/C=C/C=C2/C(=O)N(CCNC(=O)CCCCCCOc3cccc4c3C(=O)N(C3CCC(=O)NC3=O)C4=O)c3ccccc32)cc1]

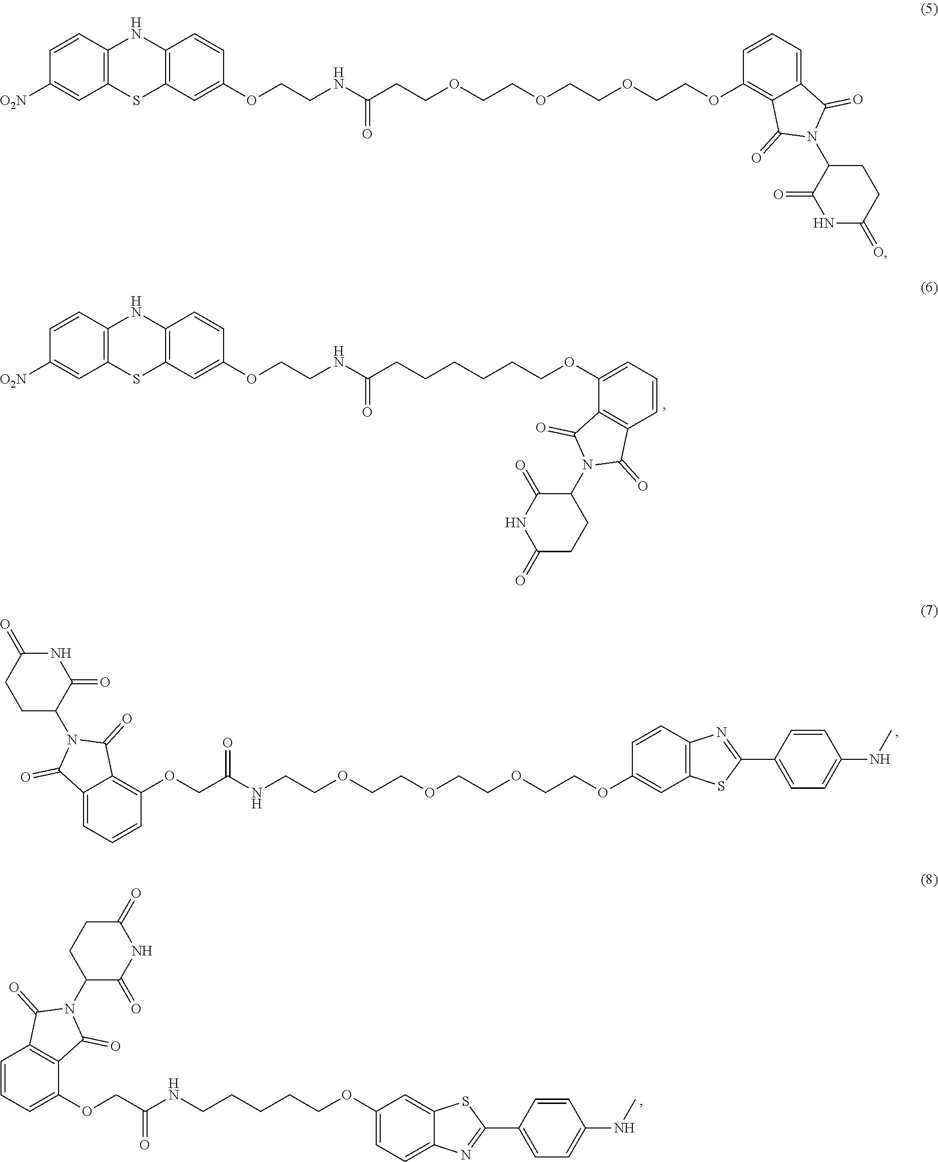 [SMILES: O=C(CCOCCOCCOCCOc1cccc2c1C(=O)N(C1CCC(=O)NC1=O)C2=O)NCCOc1ccc2c(c1)Sc1cc([N+](=O)[O-])ccc1N2 ; O=C(CCCCCCOc1cccc2c1C(=O)N(C1CCC(=O)NC1=O)C2=O)NCCOc1ccc2c(c1)Sc1cc([N+](=O)[O-])ccc1N2 ; CNc1ccc(-c2nc3ccc(OCCOCCOCCOCCNC(=O)COc4cccc5c4C(=O)N(C4CCC(=O)NC4=O)C5=O)cc3s2)cc1 ; CNc1ccc(-c2nc3ccc(OCCCCCNC(=O)COc4cccc5c4C(=O)N(C4CCC(=O)NC4=O)C5=O)cc3s2)cc1]

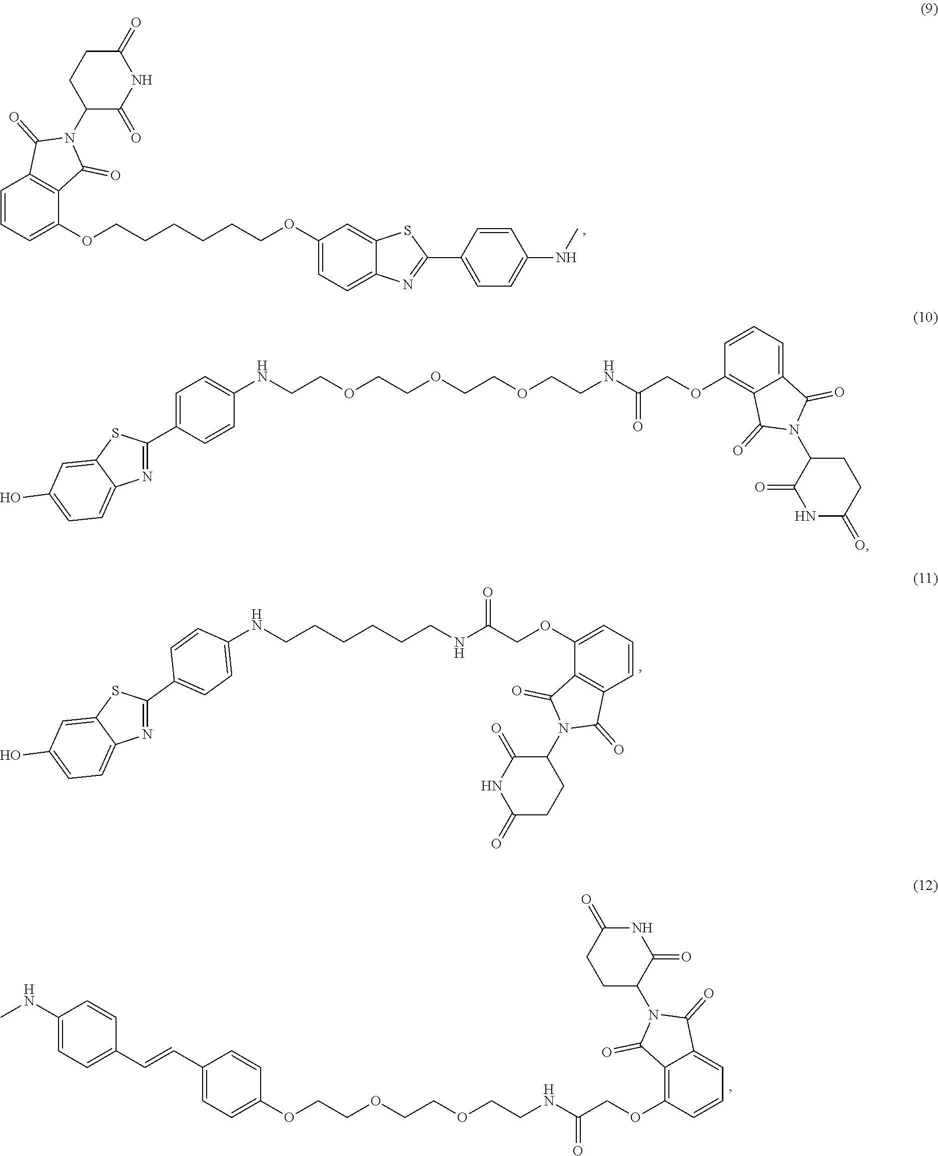

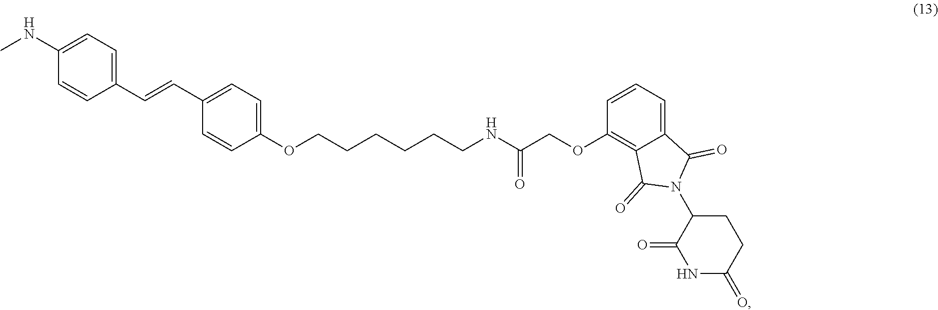 [SMILES: CNc1ccc(/C=C/c2ccc(OCCCCCCNC(=O)COc3cccc4c3C(=O)N(C3CCC(=O)NC3=O)C4=O)cc2)cc1]

or a pharmaceutically acceptable salt or stereoisomer thereof.

Bispecific compounds of formula (I) may be in the form of a free acid or free base, or a pharmaceutically acceptable salt. As used herein, the term “pharmaceutically acceptable” in the context of a salt refers to a salt of the compound that does not abrogate the biological activity or properties of the compound, and is relatively non-toxic, i.e., the compound in salt form may be administered to a subject without causing undesirable biological effects (such as dizziness or gastric upset) or interacting in a deleterious manner with any of the other components of the composition in which it is contained. The term “pharmaceutically acceptable salt” refers to a product obtained by reaction of the compound of the present invention with a suitable acid or a base. Examples of pharmaceutically acceptable salts of the compounds of this invention include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the invention can form pharmaceutically acceptable salts with various organic bases such as lysine, arginine, guanidine, diethanolamine or metformin.

Bispecific compounds of formula (I) may have at least one chiral center and thus may be in the form of a stereoisomer, which as used herein, embraces all isomers of individual compounds that differ only in the orientation of their atoms in space. The term stereoisomer includes mirror image isomers (enantiomers which include the (R-) or (S-) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R-) form is considered equivalent to administration of the compound in its (S-) form. Accordingly, the compounds of the present invention may be made and used in the form of individual isomers and substantially free of other isomers, or in the form of a mixture of various isomers, e.g., racemic mixtures of stereoisomers.

In some embodiments, the bispecific compound of formula (I) is an isotopic derivative in that it has at least one desired isotopic substitution of an atom, at an amount above the natural abundance of the isotope, i.e., enriched. In one embodiment, the compound includes deuterium or multiple deuterium atoms. Substitution with heavier isotopes such as deuterium, i.e. ²H, may afford certain therapeutic advantages resulting from greater metabolic stability, for example, increased in vivo half-life or reduced dosage requirements, and thus may be advantageous in some circumstances.

In addition to the isotopic derivatives, the term “bispecific compounds of formula (I)” embraces the use of N-oxides, crystalline forms (also known as polymorphs), active metabolites of the compounds having the same type of activity, tautomers, and unsolvated as well as solvated forms with pharmaceutically acceptable solvents such as water, ethanol, and the like, of the compounds. The solvated forms of the conjugates presented herein are also considered to be disclosed herein.

### Methods of Synthesis

In another aspect, the present invention is directed to a method for making a bispecific compound of formula (I), or a pharmaceutically acceptable salt or stereoisomer thereof. Broadly, the inventive compounds or pharmaceutically-acceptable salts or stereoisomers thereof may be prepared by any process known to be applicable to the preparation of chemically related compounds. The compounds of the present invention will be better understood in connection with the synthetic schemes that described in various working examples and which illustrate non-limiting methods by which the compounds of the invention may be prepared.

### Pharmaceutical Compositions

Another aspect of the present invention is directed to a pharmaceutical composition that includes a therapeutically effective amount of a bispecific compound of formula (I) or a pharmaceutically acceptable salt or stereoisomer thereof, and a pharmaceutically acceptable carrier. The term “pharmaceutically acceptable carrier,” as known in the art, refers to a pharmaceutically acceptable material, composition or vehicle, suitable for administering compounds of the present invention to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may also include one or more pharmaceutically acceptable excipients.

Broadly, bispecific compounds of formula (I) and their pharmaceutically acceptable salts and stereoisomers may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the mode of administration which may include enteral (e.g., oral, buccal, sublingual and rectal), parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the bispecific compound may be administered relatively quickly such as in the case of a single-dose treatment and/or an acute condition.

In some embodiments, the bispecific compounds are formulated for oral or intravenous administration (e.g., systemic intravenous injection).

Accordingly, bispecific compounds of formula (I) may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). Compounds may also be formulated for rapid, intermediate or extended release.

Solid dosage forms for oral administration include capsules, tablets, pills, powders, and granules. In such solid dosage forms, the active compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, bispecific compounds of formula (I) may be formulated in a hard or soft gelatin capsule. Representative excipients that may be used include pregelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups and elixirs. In addition to the compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include an excipients such as wetting agents, suspending agents, coloring, sweetening, flavoring, and perfuming agents.

Injectable preparations for parenteral administration may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, bispecific compounds of formula (I) may be administered in a local rather than systemic manner, for example, via injection of the conjugate directly into an organ, often in a depot preparation or sustained release formulation. In specific embodiments, long acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody. In such embodiments, the liposomes are targeted to and taken up selectively by the organ.

The compositions may be formulated for buccal or sublingual administration, examples of which include tablets, lozenges and gels.

The bispecific compounds of formula (I) may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Bispecific compounds of formula (I) may be formulated for topical administration which as used herein, refers to administration intradermally by invention of the formulation to the epidermis. These types of compositions are typically in the form of ointments, pastes, creams, lotions, gels, solutions and sprays.

Representative examples of carriers useful in formulating bispecific compounds for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents are capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerin, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of a bispecific compound of formula (I) or a pharmaceutically acceptable salt or a stereoisomer thereof that is effective in producing the desired therapeutic response in a patient suffering from a neurodegenerative disease or disorder mediated by α-synuclein protein activity. The term “therapeutically effective amount” thus includes the amount of the bispecific compound or a pharmaceutically acceptable salt or a stereoisomer thereof, that when administered, induces a positive modification in the disease or disorder to be treated, or is sufficient to prevent development or progression of the disease or disorder, or alleviate to some extent, one or more of the symptoms of the disease or disorder being treated in a subject, or which simply kills or inhibits the growth of diseased cells, or reduces the amounts of α-synuclein protein in diseased cells.

The total daily dosage of the bispecific compounds and usage thereof may be decided in accordance with standard medical practice, e.g., by the attending physician using sound medical judgment. The specific therapeutically effective dose for any particular subject may depend upon a variety of factors including the disease or disorder being treated and the severity thereof (e.g., its present status); the age, body weight, general health, sex and diet of the subject; the time of administration, route of administration, and rate of excretion of the specific compound employed; the duration of the treatment; drugs used in combination or coincidental with the bispecific compound; and like factors well known in the medical arts (see, for example, Goodman and Gilman's. The Pharmacological Basis of Therapeutics, 10th Edition, A. Gilman, J. Hardman and L. Limbird, eds., McGraw-Hill Press, 155-173, 2001).

Bispecific compounds of formula (I) and their pharmaceutically acceptable salts and stereoisomers may be effective over a wide dosage range. In some embodiments, the total daily dosage (e.g., for adult humans) may range from about 0.001 to about 1600 mg, from 0.01 to about 1600 mg, from 0.01 to about 500 mg, from about 0.01 to about 100 mg, from about 0.5 to about 100 mg, from 1 to about 100-400 mg per day, from about 1 to about 50 mg per day, and from about 5 to about 40 mg per day, or in yet other embodiments from about 10 to about 30 mg per day. In some embodiments, the total daily dosage may range from 400 mg to 600 mg. Individual dosages may be formulated to contain the desired dosage amount depending upon the number of times the compound is administered per day. By way of example, capsules may be formulated with from about 1 to about 200 mg of compound (e.g., 1, 2, 2.5, 3, 4, 5, 10, 15, 20, 25, 50, 100, 150, and 200 mg). In some embodiments, the compound may be administered at a dose in range from about 0.01 mg to about 200 mg/kg of body weight per day. In some embodiments, a dose of from 0.1 to 100, e.g., from 1 to 30 mg/kg per day in one or more dosages per day may be effective. By way of example, a suitable dose for oral administration may be in the range of 1-30 mg/kg of body weight per day, and a suitable dose for intravenous administration may be in the range of 1-10 mg/kg of body weight per day.

### Methods of Use

In some aspects, the present invention is directed to methods of treating diseases or disorders involving aberrant (e.g., dysfunctional or dysregulated) α-synuclein protein activity, that entails administration of a therapeutically effective amount of a bispecific compound of formula (I) or a pharmaceutically acceptable salt or stereoisomer thereof, to a subject in need thereof.

The present invention is directed to treating neurodegenerative diseases or disorders characterized or mediated by aberrant α-synuclein protein activity (e.g., elevated levels of α-synuclein or otherwise functionally abnormal, e.g., deregulated α-synuclein levels). A “disease” is generally regarded as a state of health of a subject wherein the subject cannot maintain homeostasis, and wherein if the disease is not ameliorated then the subject's health continues to deteriorate. In contrast, a “disorder” (or condition) in a subject is a state of health in which the subject is able to maintain homeostasis, but in which the subject's state of health is less favorable than it would be in the absence of the disorder. Left untreated, a disorder does not necessarily cause a further decrease in the subject's state of health.

The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “in need of” treatment according to the present invention may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Thus, subjects suffering from, and suspected of suffering from, a specific disease or disorder are not necessarily two distinct groups.

Exemplary neurodegenerative diseases or disorders that may be amenable to treatment with the bispecific compounds of the present invention amyotrophic lateral sclerosis, Parkinson's disease, Prion disease, epilepsy, encephalopathy, Huntington's disease, ataxia, dystonia, encephalitis, dysarthria, Alzheimer's disease, multiple system atrophy, autism, migraines, and dementia with Lewy bodies.

In some embodiments, the neurodegenerative disease is Parkinson's disease.

In some embodiments, the neurodegenerative disease is multiple system atrophy.

In some embodiments, the neurodegenerative disease is dementia with Lewy bodies.

The methods of the present invention may entail administration of a bispecific compound of formula (I) or a pharmaceutical composition thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails at least one 28-day cycle which includes daily administration for 3 weeks (21 days) followed by a 7-day “off” period. In other embodiments, the bispecific compound may be dosed twice a day (BID) over the course of two and a half days (for a total of 5 doses) or once a day (QD) over the course of two days (for a total of 2 doses). In other embodiments, the bispecific compound may be dosed once a day (QD) over the course of 5 days.

### Combination Therapy

The bispecific compounds of formula (I) and their pharmaceutically acceptable salts and stereoisomers may be used in combination or concurrently with at least one other active agent in treating neurodegenerative diseases and disorders. The terms “in combination” and “concurrently” in this context mean that the agents are co-administered, which includes substantially contemporaneous administration, by way of the same or separate dosage forms, and by the same or different modes of administration, or sequentially, e.g., as part of the same treatment regimen, or by way of successive treatment regimens. Thus, if given sequentially, at the onset of administration of the second compound, the first of the two compounds is in some cases still detectable at effective concentrations at the site of treatment. The sequence and time interval may be determined such that they can act together (e.g., synergistically to provide an increased benefit than if they were administered otherwise). For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

In some embodiments, the treatment regimen may include administration of a bispecific compound of formula (I) or a pharmaceutically acceptable salt or stereoisomer in combination with one or more additional therapeutics known for use in treating the disease or disorder. The dosage of the additional therapeutic agent may be the same or even lower than known or recommended doses. See, Hardman et al., eds., Goodman & Gilman's The Pharmacological Basis Of Basis Of Therapeutics, 10th ed., McGraw-Hill, New York, 2001; Physician's Desk Reference 60th ed., 2006.

In some embodiments, the compound of the invention and the additional therapeutic agent may be administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. The two or more therapeutic agents may be administered within the same patient visit.

In some embodiments, a bispecific compound of the present invention may be used in combination with one or more of Levodopa, Sinemet, Safinamide, Ropinirole, Pramipexole, Rotigotine Amantadine, Artane, Cogentin, Eldepryl, Zelapar, and Azilect (e.g., for Parkinson's disease. In some embodiments, a bispecific compound of the present invention may be used in combination with one or more of Aricept, Exelon, Razadyne, Namenda, and Namzaric (e.g., for Alzheimer's disease) In some embodiments, a bispecific compound of the present invention may be used in combination with one or more of Xenazine, Haldol, chlorpromazine, Risperdal, Seroquel, Keppra, Klonopin, Celexa, Prozac, Epitol, and Depacon (e.g., for Huntington's disease). In some embodiments, a bispecific compound of the present invention may be used in combination with one or more of trazodone, Zoloft, Luvox, Zyprexa, and Seroquel (e.g., for Pick's syndrome). Representative examples of other active agents known to treat neurodegenerative diseases and disorders include dopaminergic treatments (e.g., Carbidopa-levodopa, pramipexole (Mirapex), ropinirole (Requip) and rotigotine (Neupro, given as a patch)). Apomorphine and monoamine oxidase B (MAO-B) inhibitors (e.g., selegiline (Eldepryl, Zelapar), rasagiline (Azilect) and safinamide (Xadago)) for PD and movement disorders, cholinesterase inhibitors for cognitive disorders (e.g., benztropine (Cogentin) or trihexyphenidyl), antipsychotic drugs for behavioral and psychological symptoms of dementia, as well as agents aimed to slow the development of diseases, such as Riluzole for ALS, cerebellar ataxia and Huntington's disease, non-steroidal anti-inflammatory drugs for Alzheimer's disease, and caffeine A2A receptor antagonists and CERE-120 (adeno-associated virus serotype 2-neurturin) for the neuroprotection of Parkinson's disease.

### Pharmaceutical Kits

The present bispecific compounds and/or compositions containing them may be assembled into kits or pharmaceutical systems. Kits or pharmaceutical systems according to this aspect of the invention include a carrier or package such as a box, carton, tube or the like, having in close confinement therein one or more containers, such as vials, tubes, ampoules, or bottles, which contain a bispecific compound of formula (I) or a pharmaceutical composition thereof. The kits or pharmaceutical systems of the invention may also include printed instructions for using the compounds and compositions.

These and other aspects of the present invention will be further appreciated upon consideration of the following Examples, which are intended to illustrate certain particular embodiments of the invention but are not intended to limit its scope, as defined by the claims.

### EXAMPLES

### Example 1: Synthesis of 2-(4-(methylamino)phenyl)benzo[d]thiazol-6-ol

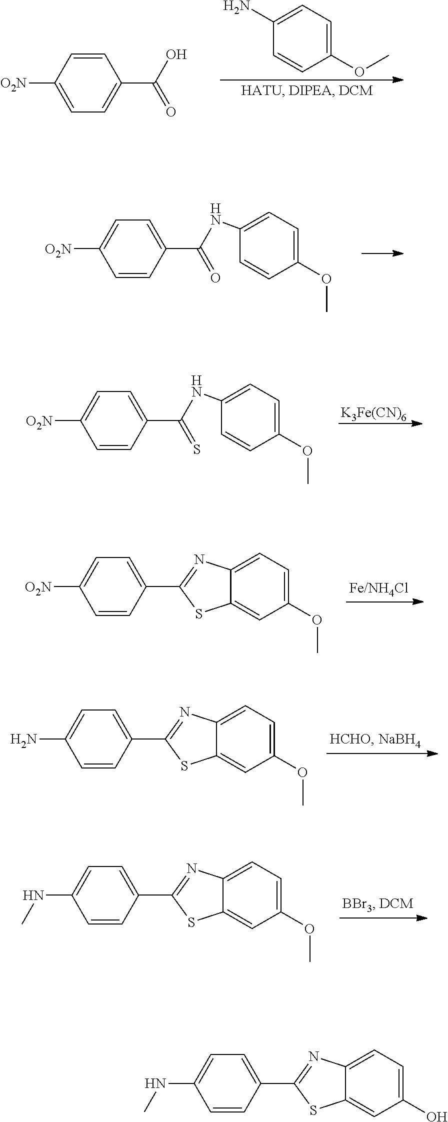

### N-(4-methoxyphenyl)-4-nitrobenzamide

A mixture of 4-nitrobenzoic acid (30.0 g, 0.18 mol), 4-methoxyaniline (22.1 g, 0.18 mol), HATU (81.9 g, 0.22 mol) and DIPEA (46.4 g, 0.36 mol) in DCM (2.0 L) was stirred at room temperature (rt). After 16 hours, liquid chromatography-mass spectrometry (LCMS) showed full conversion of starting materials. The mixture was washed with diluted HCl solution (2 M, 800 mL), saturated NaHCO₃ solution (1000 mL) and brine (500 mL), dried over anhydrous Na₂SO₄, filtered, concentrated and purified by column chromatography (silica gel, DCM:MeOH=10:1) to yield the title compound as a yellow solid (45 g, yield 92%). LCMS (m/z): 273 [M+H]⁺.

### N-(4-methoxyphenyl)-4-nitrobenzothioamide

A mixture of N-(4-methoxyphenyl)-4-nitrobenzamide (23 g, 80 mmol) and Lawesson reagent (16.12 g, 40 mmol) in toluene (200 mL) was stirred at 110° C. After 16 hours, LCMS showed full conversion of starting materials. The mixture was concentrated to remove the organic solvent, the residue was diluted with DCM (500 mL), washed with diluted HCl solution (1 M, 500 mL), saturated NaHCO₃ solution (800 mL) and brine (500 mL), dried over anhydrous Na₂SO₄, filtered, concentrated and purified with column chromatography (silica gel, PE:EtOAc=2:1) to yield the title compound as a yellow solid (17 g, yield 59%). LCMS (m/z): 289 [M+H]⁺.

### 6-methoxy-2-(4-nitrophenyl)benzo[d]thiazole

A mixture of N-(4-methoxyphenyl)-4-nitrobenzothioamide (20 g, 103 mmol) and K₃Fe(CN)₆ (133.61 g, 412 mmol) in NaOH solution (10%, 800 mL) and EtOH (30 mL) was heated at reflux. After 16 hours, LCMS showed full conversion of starting materials. The mixture was cooled to room temperature (rt), diluted with saturated Na₂SO₃ solution (200 mL) and extracted with DCM (500 mL×2), the combined organic was washed with brine (500 mL), dried over anhydrous Na₂SO₄, filtered, concentrated and purified with column chromatography (silica gel, DCM:MeOH=10:1) to yield the title compound as a yellow solid (9.2 g, yield 31%). LCMS (m/z): 286.9 [M+H]⁺.

### 4-(6-methoxybenzo[d]thiazol-2-yl)aniline

A mixture of 6-methoxy-2-(4-nitrophenyl)benzo[d]thiazole (8.1 g, 28.3 mmol), Fe powder (7.9 g, 141.4 mmol), NH₄Cl (9.08 g, 169.7 mmol) and concentrated HCl solution (14.15 mL, 169.7 mmol) in EtOH (500 mL) was stirred at 80° C. After 16 hours, LCMS showed full conversion of starting materials. The mixture was cooled to rt and filtered through a pad of Celite®. The filtrate was concentrated and purified with column chromatography (silica gel, DCM:MeOH=30:1) to yield the title compound as a brown solid (8.2 g, yield 117%). LCMS (m/z): 257 [M+H]⁺.

### 4-(6-methoxybenzo[d]thiazol-2-yl)aniline

A mixture of 4-(6-methoxybenzo[d]thiazol-2-yl)aniline (6.1 g, 23.8 mmol), (HCHO). (4.28 g, 142.8 mmol) and MeONa (7.71 g, 142.8 mmol) in MeOH (500 mL) was stirred at reflux for 2 hours, and then NaBH₄ (7.2 g, 190.4 mmol) was added, the mixture was stirred at rt for 30 minutes, and then heated to reflux. After 16 hours, LCMS showed full conversion of starting materials. The mixture was cooled to rt, diluted with water (1000 mL) and extracted with DCM (500 mL×3). The combined organic layers were dried over anhydrous Na₂SO₄, filtered and concentrated to yield the crude title compound as a brown solid (6.8 g, yield 105%), which was used directly in the next step without purification. LCMS (m/z): 271 [M+H]⁺.

### 2-(4-(methylamino)phenyl)benzo[d]thiazol-6-ol

A mixture of 4-(6-methoxybenzo[d]thiazol-2-yl)aniline (800 mg, 2.96 mmol) and BBr₃ (1 M in DCM, 29.6 mL, 29.6 mmol) in DCE (500 mL) was stirred at −10° C. for 30 minutes, and then stirred at rt. After 3 hours, LCMS showed full conversion of starting materials. The mixture was concentrated in vacuo. The residue was treated with saturated NaHCO₃ solution until a pH 8 was reached and the resulting mixture was extracted with DCM (100 mL×2). The combined organic layers were washed with brine (300 mL), dried over anhydrous Na₂SO₄, concentrated and purified with column chromatography (silica gel, DCM:MeOH=10:1) to yield the title compound as a brown solid (500 mg, yield 66%). ¹H NMR (DMSO-d₆, 400 MHz): δ 7.72 (br, 3H), 7.31 (br, 1H), 6.91 (br, 1H), 6.63 (br, 2H), 6.36 (br, 1H), 2.74 (br, 3H).

LCMS (m/z): 257.1 [M+H]⁺.

### Example 2: Synthesis of 2-(2,6-dioxopiperidin-3-yl)-4-(6-(2-(4-(methylamino)phenyl)benzo[d]thiazol-6-yloxy)hexyloxy)isoindoline-1,3-dione (9)

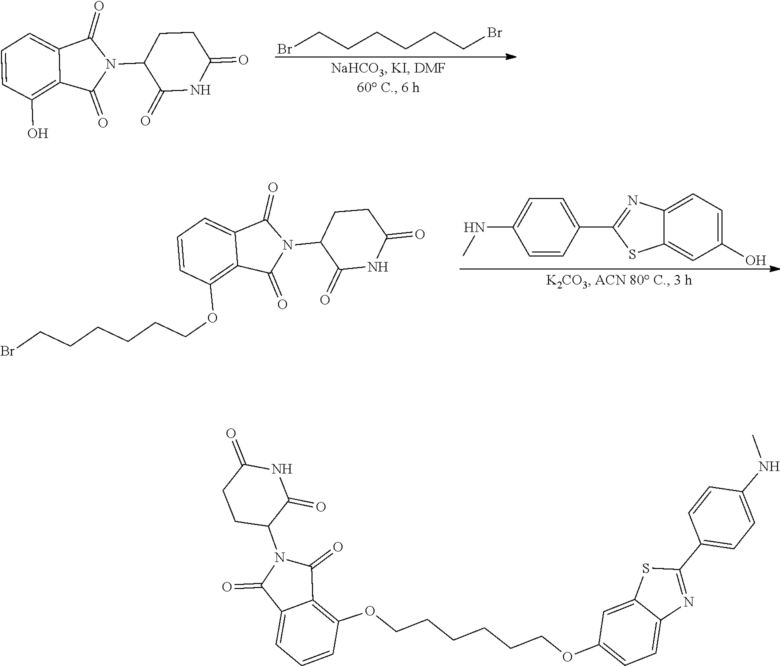 [SMILES: O=C1CCC(N2C(=O)c3cccc(O)c3C2=O)C(=O)N1 ; BrCCCCCCBr ; O=C1CCC(N2C(=O)c3cccc(OCCCCCCBr)c3C2=O)C(=O)N1 ; CNc1ccc(-c2nc3ccc(O)cc3s2)cc1 ; CNc1ccc(-c2nc3ccc(OCCCCCCOc4cccc5c4C(=O)N(C4CCC(=O)NC4=O)C5=O)cc3s2)cc1]

### 4-(6-bromohexyloxy)-2-(2,6-dioxopiperidin-3-yl)isoindoline-1,3-dione

A mixture of 2-(2,6-dioxopiperidin-3-yl)-4-hydroxyisoindoline-1,3-dione (440 mg, 1.61 mmol), 1,6-dibromohexane (1176 mg, 4.82 mmol), NaHCO₃ (676 mg, 8.05 mmol) and KI (134 mg, 0.81 mmol) in DMF (21 mL) was stirred at 60° C. After 6 hours, LCMS showed full conversion of starting materials. The mixture was diluted with water (200 mL) and extracted with DCM (100 mL×2), the combined organic was dried over anhydrous Na₂SO₄, filtered, concentrated and purified with column chromatography (silica gel, DCM:MeOH=30:1) to yield the title compound as a brown oil (576 mg yield 82%). LCMS (m/z): 438 [M+H]⁺.

### 2-(2,6-dioxopiperidin-3-yl)-4-(6-(2-(4-(methylamino)phenyl)benzo[d]thiazol-6-yloxy)hexyloxy)isoindoline-1,3-dione

A mixture of 4-(6-bromohexyloxy)-2-(2,6-dioxopiperidin-3-yl)isoindoline-1,3-dione (576 mg, 1.32 mmol), 2-(4-(methylamino)phenyl)benzo[d]thiazol-6-ol (240 mg, 0.94 mmol) and K₂CO₃ (389 mg, 2.82 mmol) in MeCN (21 mL) was stirred at 80° C. After 3 hours, LCMS showed full conversion of starting materials. The mixture was concentrated to remove the organic solvent. The residue was diluted with water (100 mL) and extracted with DCM (100 mL×2), and the combined organic layers were dried over anhydrous Na₂SO₄, filtered, concentrated and purified with preparative HPLC (C18 column, CH₃CN/H₂O, containing 0.05% TFA) to yield the title compound as a yellow solid (8.8 mg). ¹H NMR (DMSO-d₆, 400 MHz): δ 8.43 (br, 1H), 7.78-7.74 (m, 5H), 7.57 (d, J=4.0 Hz, 1H), 7.51 (d, J=8.0 Hz, 1H), 7.43 (d, J=4.0 Hz, 1H), 7.03 (dd, J=12.0, 4.0 Hz, 1H), 6.64 (d, J=8.0 Hz, 2H), 6.38 (d, J=4.0 Hz, 1H), 5.32 (t, J=4.0 Hz, 1H), 5.07 (dd, J=12.0, 4.0 Hz, 1H), 4.22 (t, J=6.0 Hz, 3H), 4.04 (t, J=6.0 Hz, 2H), 2.75 (d, J=8.0 Hz, 3H), 2.00 (m, 3H), 1.79 (br, 4H), 1.53-1.30 (m, 4H), 0.85 (t, J=6.0 Hz, 1H). LCMS (m/z): 612.9 [M+H]⁺.

### Example 3: Synthesis of 2-(2-(2,6-dioxopiperidin-3-yl)-1,3-dioxoisoindolin-4-yloxy)-N-(2-(2-(2-(2-(2-(4-(methylamino)phenyl) benzo[d]thiazol-6-yloxy)ethoxy)ethoxy)ethoxy)ethyl)acetamide (7)

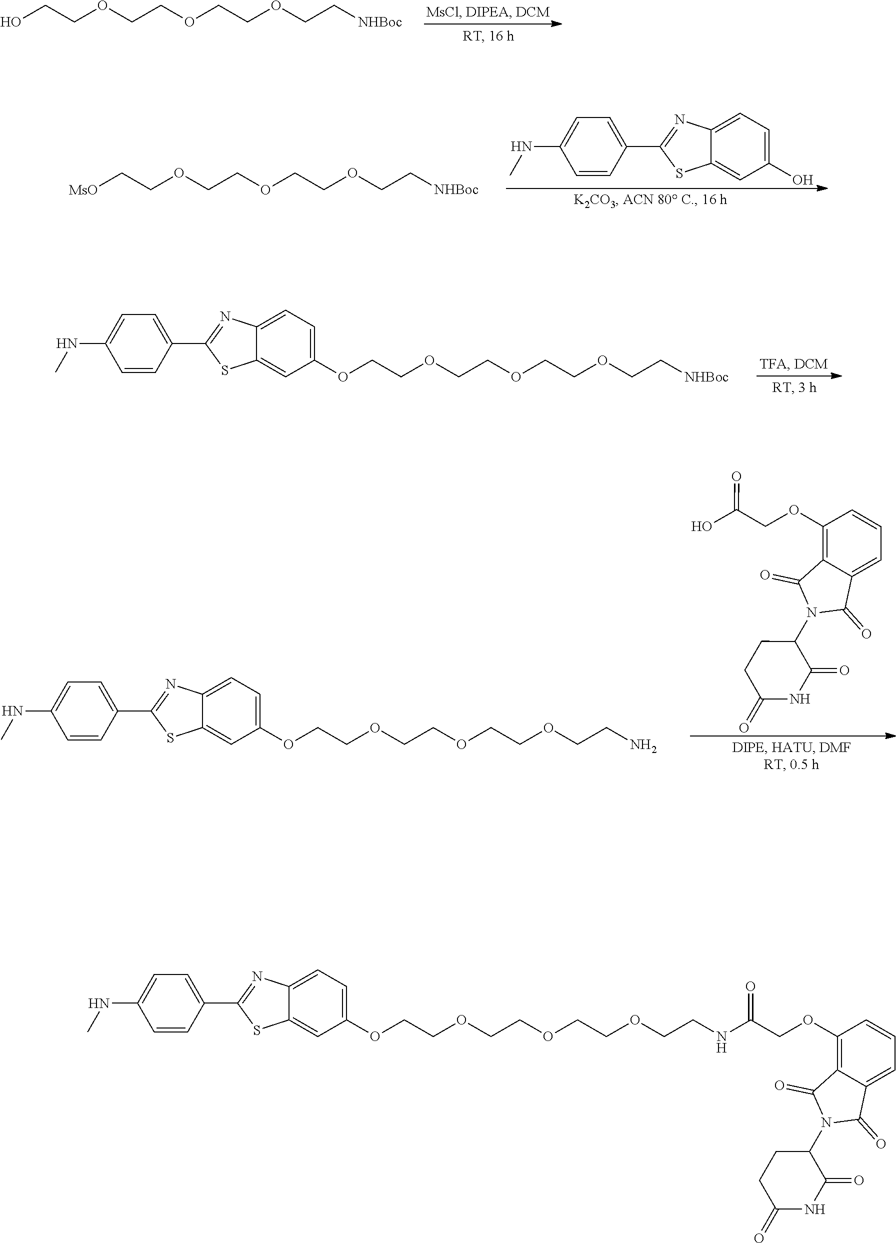

### 2,2-dimethyl-4-oxo-3,8,11,14-tetraoxa-5-azahexadecan-16-yl methanesulfonate

A mixture of tert-butyl (2-(2-(2-(2-hydroxyethoxy)ethoxy)ethoxy)ethyl)carbamate (500 mg, 1.71 mmol), MsCl (391 mg, 3.41 mmol) and DIPEA (662 mg, 5.13 mmol) in DCM (20 mL) was stirred at rt for 16 hours. The mixture was then diluted with DCM (200 mL), washed with saturated Na₂CO₃ solution (150 mL) and brine (150 mL×2), dried over anhydrous Na₂SO₄, filtered and concentrated to yield crude title compound as a yellowish oil (538 mg, yield 85%), which was used directly in the next step without further purification. LCMS (m/z): 272 [M+H−100]⁺, 394 [M+Na]⁺.

### tert-Butyl 2-(2-(2-(2-(2-(4-(methylamino)phenyl)benzo[d]thiazol-6-yloxy)ethoxy)ethoxy)ethoxy)ethylcarbamate

A mixture of 2,2-dimethyl-4-oxo-3,8,11,14-tetraoxa-5-azahexadecan-16-yl methanesulfonate (538 mg, 1.45 mmol), 2-(4-(methylamino)phenyl)benzo[d]thiazol-6-ol (120 mg, 0.47 mmol) and K₂CO₃ (200 mg, 1.45 mmol) in MeCN (25 mL) was stirred at 80° C. for 16 hours. The mixture was then diluted with DCM (200 mL), washed with brine (200 mL×2), dried over anhydrous Na₂SO₄, filtered and concentrated in vacuo. The residue was purified by column chromatography (silica gel, PE:EA=10:1-1:1) to yield the title compound as a brown solid (220 mg, 90%). LCMS (m/z): 532 [M+H]⁺.

### 4-(6-(2-(2-(2-(2-aminoethoxy)ethoxy)ethoxy)ethoxy)benzo[d]thiazol-2-yl)-N-methylaniline

A mixture of tert-butyl 2-(2-(2-(2-(2-(4-(methylamino)phenyl)benzo[d]thiazol-6-yloxy)ethoxy)ethoxy)ethoxy)ethylcarbamate (220 mg, 0.41 mmol) and TFA (804 mg, 8.29 mmol) in DCM (12 mL) was stirred at rt for 4 hours. The mixture was then diluted with DCM (100 mL), washed with saturated Na₂CO₃ solution (100 mL) and brine (100 mL), dried over anhydrous Na₂SO₄, filtered and concentrated in vacuo to yield the crude title compound as a yellow solid (150 mg, yield 84%), which was used directly in the next step without further purification. LCMS (m/z): 432 [M+H]⁺.

### 2-(2-(2,6-dioxopiperidin-3-yl)-1,3-dioxoisoindolin-4-yloxy)-N-(22-(2-(2-(2-(4-(methylamino)phenyl) benzo[d]thiazol-6-yloxy)ethoxy)ethoxy)ethoxy)ethyl)acetamide

A mixture of 4-(6-(2-(2-(2-(2-aminoethoxy)ethoxy)ethoxy)ethoxy)benzo[d]thiazol-2-yl)-N-methylaniline (150 mg, 0.35 mmol), 2-((2-(2,6-dioxopiperidin-3-yl)-1,3-dioxoisoindolin-4-yl)oxy)acetic acid (139 mg, 0.42 mmol), HATU (265 mg, 0.70 mmol) and DIPEA (225 mg, 1.74 mmol) in DMF (12 mL) was stirred at rt for 30 minutes. The mixture was then diluted with brine (100 mL) and extracted with DCM (100 mL×2). The combined organic layers were dried over anhydrous Na₂SO₄, filtered and concentrated in vacuo. The residue was purified with preparative HPLC (C18 column, CH₃CN/H₂O, containing 0.05% TFA) to yield the title compound as an off-white solid (65.8 mg). ¹H NMR (DMSO-d₆, 400 MHz): δ 11.11 (s, 1H), 7.99 (t, J=6.0 Hz, 1H), 7.82-7.74 (m, 4H), 7.59 (d, J=4.0 Hz, 1H), 7.48 (d, J=8.0 Hz, 1H), 7.38 (d, J=8 Hz, 1H), 7.04 (dd, J=8.0, 4.0 Hz, 1H), 6.63 (d, J=8.0 Hz, 2H), 6.39 (d, J=8.0 Hz, 1H), 5.11 (dd, J=12.0, 4.0 Hz, 1H), 4.78 (s, 2H), 4.16-4.14 (m, 2H), 3.78-3.70 (m, 2H), 3.65-3.40 (m, 12H), 2.92-2.85 (m, 1H), 2.75 (d, J=8.0 Hz, 3H), 2.60-2.50 (m, 2H), 2.08-2.00 (m, 1H). LCMS (m/z): 745.9 [M+H]⁺.

### Example 4: Synthesis of 2-(2-(2,6-dioxopiperidin-3-yl)-1,3-dioxoisoindolin-4-yloxy)-N-(5-(2-(4-(methylamino)phenyl)benzo[d]-thiazol-6-yloxy)pentyl)acetamide (8)

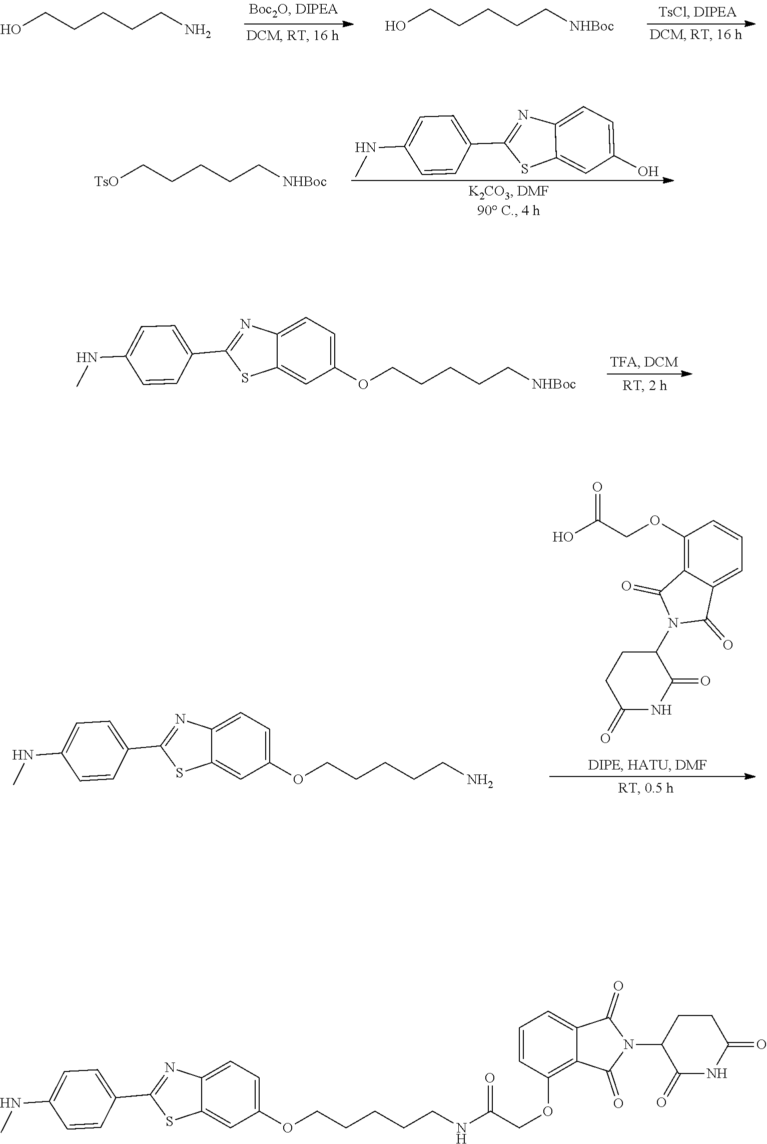

### tert-Butyl 5-hydroxypentylcarbamate

A mixture of 5-aminopentan-1-ol (2100 mg, 20.36 mmol), Boc₂O (6664 mg, 30.54 mmol) and DIPEA (7898 mg, 61.08 mmol) in DCM (35 mL) was stirred at rt for 16 hours. The mixture was then diluted with DCM (200 mL), washed with saturated Na₂CO₃ solution (150 mL) and brine (150 mL×2), dried over anhydrous Na₂SO₄, filtered and concentrated to yield the crude title compound as a brown oil (4.5 g), which was used directly in the next step without purification. LCMS (m/z): 226 [M+Na]⁺.

### tert-Butyl 5-hydroxypentylcarbamate

A mixture of tert-butyl 5-hydroxypentylcarbamate (4.50 g, 22.17 mmol), TsCl (6.34 g, 33.25 mmol) and DIPEA (8.60 mg, 66.51 mmol) in DCM (65 mL) was stirred at rt for 16 hours. The mixture was concentrated in vacuo and the residue was diluted with DCM (200 mL), washed with brine (200 mL×2), dried over anhydrous Na₂SO₄, filtered and concentrated in vacuo. The residue was purified by column chromatography (silica gel, PE:EA=10:1-1:10) to yield the title compound as a yellow oil (2.3 g, 30%) LCMS (m/z): 258 [M+H−100]⁺, 380 [M+Na]⁺.

### tert-Butyl 5-(2-(4-(methylamino)phenyl)benzo[d]thiazol-6-yloxy)pentylcarbamate

A mixture of tert-Butyl 5-hydroxypentylcarbamate (335 mg, 0.94 mmol), 2-(4-(methylamino)phenyl)benzo[d]thiazol-6-ol (120 mg, 0.47 mmol) and K₂CO₃ (195 mg, 1.41 mmol) in DMF (12 mL) was stirred at 90° C. for 4 hours. The mixture was concentrated in vacuum and the residue was diluted with DCM (100 mL), washed with brine (100 mL×3), dried over anhydrous Na₂SO₄, filtered and concentrated in vacuo to yield the crude title compound as a faint yellow solid (167 mg, yield 80%), which was used directly in the next step without further purification. LCMS (m/z): 442 [M+H]⁺.

### 4-(6-(5-aminopentyloxy)benzo[d]thiazol-2-yl)-N-methylaniline

A mixture of tert-Butyl 5-(2-(4-(methylamino)phenyl)benzo[d]thiazol-6-yloxy)pentylcarbamate (167 mg, 0.38 mmol) and TFA (433 mg, 3.8 mmol) in DCM (35 mL) was stirred at rt for 2 hours. The mixture was concentrated in vacuum and the residue was diluted with DCM (100 mL), washed with saturated Na₂CO₃ solution (100 mL) and brine (100 mL), dried over anhydrous Na₂SO₄, filtered and concentrated in vacuo to yield the crude title compound as a brown solid (105 mg, yield 81%), which was used directly in the next step without further purification. LCMS (m/z): 342 [M+H]⁺.

### 2-(2-(2,6-dioxopiperidin-3-yl)-1,3-dioxoisoindolin-4-yloxy)-N-(5-(2-(4-(methylamino)phenyl)benzo[d] thiazol-6-yloxy)pentyl)acetamide

A mixture of 4-(6-(5-aminopentyloxy)benzo[d]thiazol-2-yl)-N-methylaniline (105 mg, 0.31 mmol), 2-((2-(2,6-dioxopiperidin-3-yl)-1,3-dioxoisoindolin-4-yl)oxy)acetic acid (134 mg, 0.40 mmol), HATU (236 mg, 0.62 mmol) and DIPEA (121 mg, 0.93 mmol) in DMF (12 mL) was stirred at rt for 30 minutes. The mixture was diluted with brine (100 mL) and extracted with DCM (100 mL×). The combined organic layers were dried over anhydrous Na₂SO₄, filtered and concentrated in vacuo. The residue was purified by preparative HPLC (C18 column, CH₃CN/H₂O, containing 0.05% TFA) to yield the title compound as a yellow solid (53.3 mg). ¹H NMR (DMSO-d₆, 400 MHz): δ 11.12 (s, 1H), 8.00 (t, J=6.0 Hz, 1H), 7.85-7.70 (m, 4H), 7.58 (d, J=4.0 Hz, 1H), 7.48 (d, J=4.0 Hz, 1H), 7.40 (d, J=8 Hz, 1H), 7.03 (dd, J=8.0, 4.0 Hz, 1H), 6.64 (d, J=8.0 Hz, 2H), 6.40 (d, J=8.0 Hz, H), 5.12 (dd, J=12.0, 4.0 Hz, 1H), 4.78 (s, 2H), 4.02 (t, J=6.0 Hz, 2H), 3.20-3.17 (m, 2H), 2.90-2.80 (m, 1H), 2.75 (d, J=8.0 Hz, 3H), 2.60-2.50 (m, 2H), 2.04-2.02 (m, 1H), 1.77-1.73 (m, 2H), 1.55-1.43 (m, 4H).

### Example 5: Synthesis of 2-((2-(2,6-dioxopiperidin-3-yl)-1,3-dioxoisoindolin-4-yl)oxy)-N-(2-(2-(2-(2-((4-(6-hydroxybenzo[d]thiazol-2-yl)phenyl)amino)ethoxy)ethoxy)ethoxy)ethyl)acetamide (10)

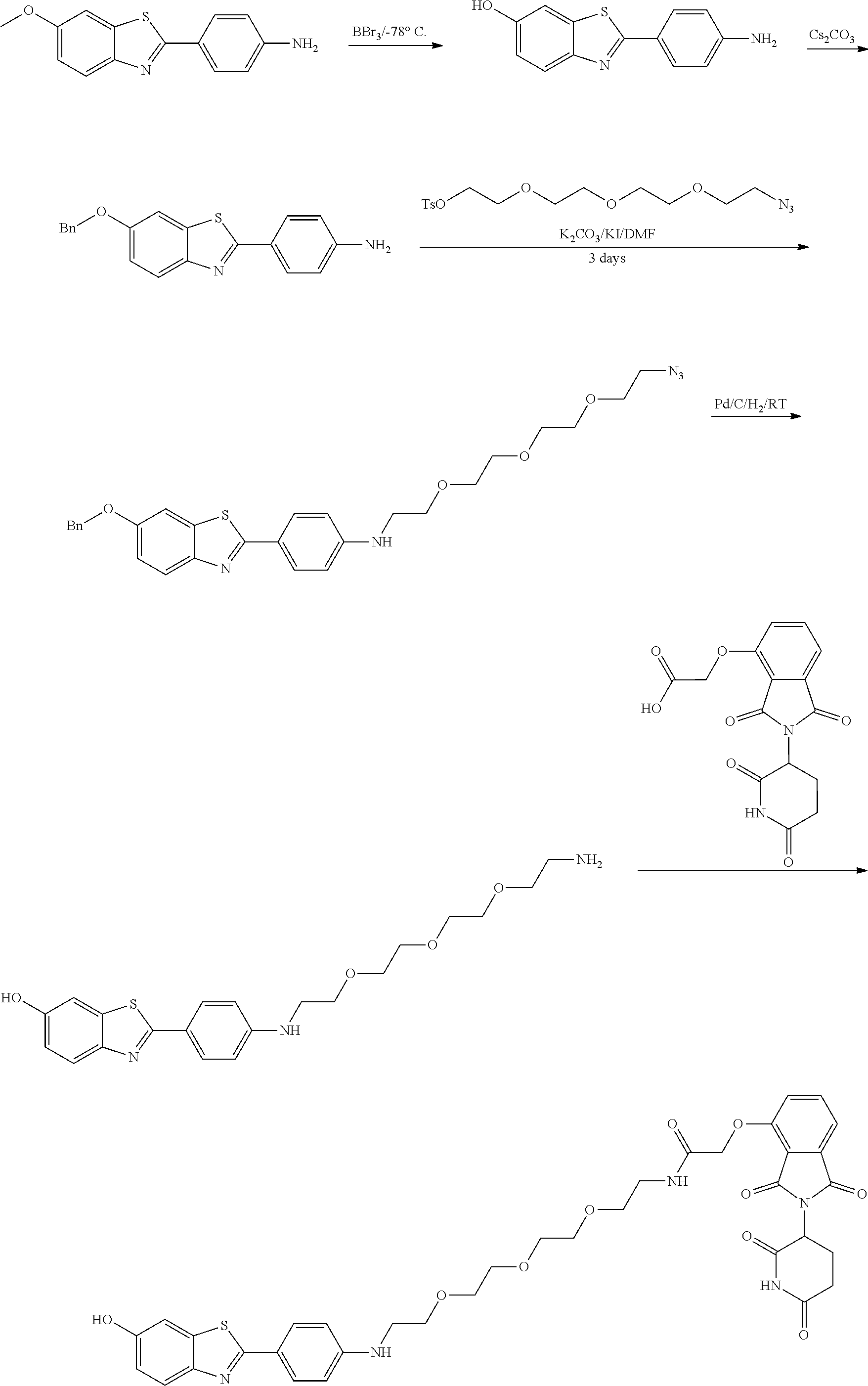

A mixture of compound 4-(6-methoxybenzo[d]thiazol-2-yl)aniline (2 g, 7.8 mmol) in DCM (40 mL) was added dropwise to BBr₃ (39 mL, 1 mol/L) at −78° C. The mixture was stirred at rt for 16 hours and concentrated in vacuo. The residue was portioned between EtOAc (100 mL) and water (100 mL). The aqueous layers were extracted with EtOAc (300 mL×2), and the combined organic layers were dried over anhydrous Na₂SO₄, filtered and concentrated. The residue was purified by CombiFlash® (MeOH:DCM=1:20) to yield 2-(4-aminophenyl)benzo[d]thiazol-6-ol as a yellow solid (1.2 g, 64%). LCMS (ESI): m/z=243.1 (M+H)⁺; RT=1.48 min.

A mixture of 2-(4-aminophenyl)benzo[d]thiazol-6-ol (1 g, 4.1 mmol), Cs₂CO₃ (2.66 g, 8.2 mmol in DMF (10 mL) was added dropwise to BnBr (701 mg, 4.1 mmol) at rt. The mixture stirred at rt for 16 hours. The mixture was portioned between EtOAc (50 mL) and water (50 mL). The aqueous layers were extracted with EtOAc (100 mL×2), and the combined organic layers were dried over anhydrous Na₂SO₄, filtered and concentrated. The residue was purified by CombiFlash® (MeOH:DCM=1:20) to yield 4-(6-(benzyloxy)benzo[d]thiazol-2-yl)aniline as a yellow solid (1.2 g, 87%). LCMS (ESI): mu/z=333.1 (M+H)⁺; RT=1.95 min.

A mixture of 4-(6-(benzyloxy)benzo[d]thiazol-2-yl)aniline (1.2 g, 3.6 mmol), KI (1.2 g, 7.2 mmol), azide-PEG4-Tos (7.2 mmol) and K₂CO₃ (994 mg, 7.2 mmol) in DMF (10 mL) stirred at rt for 3 days. The mixture was portioned between EtOAc (50 mL) and water (50 mL). The aqueous layers were extracted with EtOAc (100 mL×2), and the combined organic layers were dried over anhydrous Na₂SO₄, filtered and concentrated. The residue was purified by CombiFlash® (MeOH:DCM=1:20) to yield N-(2-(2-(2-(2-azidoethoxy)ethoxy)ethoxy)ethyl)-4-(6-(benzyloxy)benzo[d]thiazol-2-yl)aniline as a yellow solid (0.6 g, 31%). LCMS (ESI): m/z=534.2 (M+H)⁺; RT=2.07 min.

Pd/C (10%, 0.2 g) was added to a mixture of N-(2-(2-(2-(2-azidoethoxy)ethoxy)ethoxy)ethyl)-4-(6-(benzyloxy)benzo[d]thiazol-2-yl)aniline (0.6 g, 1.13 mmol) in MeOH (20 mL). The mixture was stirred at rt under H₂ (1 atm) for 16 hours and filtered. The filtrate was concentrated and the crude material was purified by CombiFlash® (MeOH:DCM=1:20) to yield 2-(4-((2-(2-(2-(2-aminoethoxy)ethoxy)ethoxy)ethyl)amino)phenyl)benzo[d]thiazol-6-ol as a yellow solid (0.3 g, 31%). LCMS (ESI): m/z=417.2 (M+H)⁺; RT=1.39 min.

A mixture of 2-(4-((2-(2-(2-(2-aminoethoxy)ethoxy)ethoxy)ethyl)amino)phenyl)benzo[d]thiazol-6-ol (0.1 g, 0.24 mmol), 2-((2-(2,6-dioxopiperidin-3-yl)-1,3-dioxoisoindolin-4-yl)oxy)acetic acid (80 mg, 0.24 mmol), EDCI (46 mg, 0.24 mmol), HOBT (36 mg, 0.26 mmol) in DMF (2 mL) was stirred at rt overnight. Water (50 mL) was added to the reaction mixture and the reaction mixture was filtered to yield the title compound as a yellow solid (20 mg, 11%). ¹H NMR (400 MHz, DMSO) δ 11.14 (s, 1H), 9.70 (s, 1H), 8.01 (s, 1H), 7.87-7.75 (m, 1H), 7.70 (dd, J=8.7, 3.2 Hz, 3H), 7.49 (d, J=7.2 Hz, 1H), 7.39 (d, J=8.6 Hz, 1H), 7.31 (d, J=2.3 Hz, 1H), 6.90 (dd, J=8.7, 2.4 Hz, 1H), 6.68 (d, J=8.7 Hz, 2H), 6.34 (s, 1H), 5.12 (dd, J=12.8, 5.3 Hz, 1H), 4.78 (s, 2H), 3.60-3.51 (m, 10H), 3.46-3.36 (m, 2H), 3.29 (dd, J=21.2, 5.5 Hz, 4H), 2.88 (dd, J=21.7, 9.9 Hz, 1H), 2.67-2.54 (m, 2H), 2.09-1.97 (m, 1H). LCMS (ESI): m/z=732.1 (M+H)⁺; RT=1.71 min.

### Example 6: Synthesis of 2-((2-(2,6-dioxopiperidin-3-yl)-1,3-dioxoisoindolin-4-yl)oxy)-N-(6-((4-(6-hydroxybenzo[d]thiazol-2-yl)phenyl)amino)hexyl)acetamide (11)

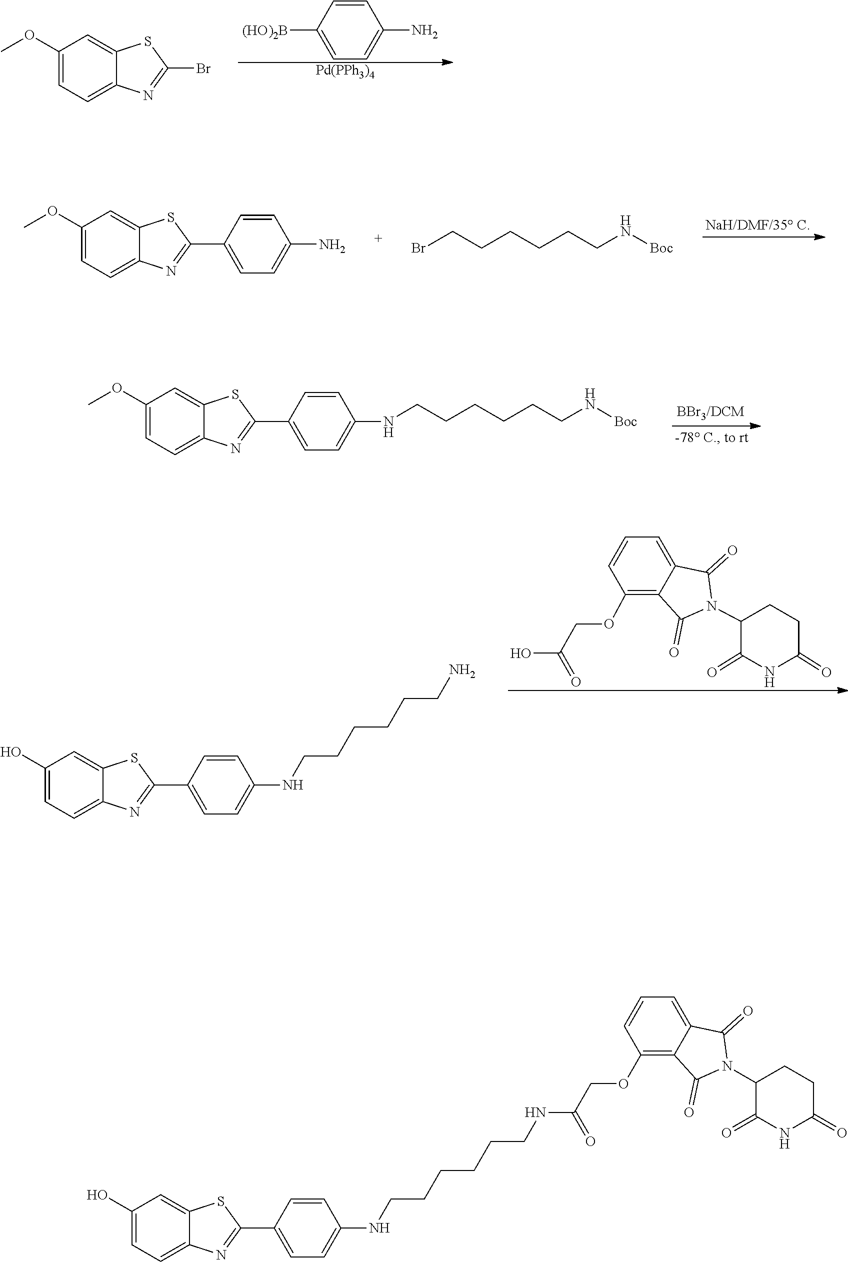

A mixture of 2-bromo-6-methoxybenzo[d]thiazole (10 g, 41.1 mmol), (4-aminophenyl)boronic acid (6.76 g, 49.32 mmol), Na₂CO₃ (11.34 g, 82.2 mmol) and Pd(PPh₃)₄ (2.36 g, 2.05 mmol) in 1,4-dioxane was heated to 100° C. for 18 hours under a N₂ atmosphere. The mixture was portioned between EtOAc (500 mL) and water (100 mL). The aqueous layers were extracted with EtOAc (300 mL×2), and the combined organic layers were dried over anhydrous Na₂SO₄, filtered and concentrated. The residue was purified by CombiFlash® (PE:EtOAc=1:20 to 100% EtOAc) to yield 4-(6-methoxybenzo[d]thiazol-2-yl)aniline as a yellow solid (7 g, 66%). LCMS (ESI): m/z=257.2 (M+H)⁺; RT=1.30 min, purity=90%.

NaH (280 mg, 11.7 mmol) was added to a solution of 4-(6-methoxybenzo[d]thiazol-2-yl)aniline (1 g, 3.90 mmol) in DMF at rt. After 30 minutes, tert-butyl (6-bromohexyl)carbamate (1.30 g, 4.68 mmol) was added, and the reaction mixture was stirred for 18 hours. The mixture was portioned between EtOAc (500 mL) and water (100 mL). The aqueous layers were extracted with EtOAc (300 mL×2), and the combined organic layers were dried over anhydrous Na₂SO₄, filtered and concentrated. The residue was purified by CombiFlash® (PE:EtOAc=1:20 to 100% EtOAc) to yield tert-butyl (6-((4-(6-methoxybenzo[d]thiazol-2-yl)phenyl)amino)hexyl)carbamate as a yellow solid (250 mg, 10%). LCMS (ESI): m/z=456.2 (M+H)⁺; RT=1.30 min, purity=80%.

BBr₃ (280 mg, 9.6 mmol) was added to a solution of tert-butyl (6-((4-(6-methoxybenzo[d]thiazol-2-yl)phenyl)amino)hexyl)carbamate (250 mg, 0.54 mmol) in DCM at −78° C. The mixture was stirred at rt for 16 hours and concentrated in vacuo. The mixture was portioned between EtOAc (500 mL) and water (100 mL). The aqueous layers were extracted with EtOAc (300 mL×2), and the combined organic layers were dried over anhydrous Na₂SO₄, filtered and concentrated. The residue was purified by CombiFlash® (PE:EtOAc=1:20 to 100% EtOAc) to yield 2-(4-((6-aminohexyl)amino)phenyl)benzo[d]thiazol-6-ol as a yellow solid (80 mg, 44%). LCMS (ESI): m/z=341.2 (M+H)⁺; RT=1.30 min, purity=90%.

A mixture of 2-(4-((6-aminohexyl)amino)phenyl)benzo[d]thiazol-6-ol (80 mg, 0.23 mmol), 2-((2-(2,6-dioxopiperidin-3-yl)-1,3-dioxoisoindolin-4-yl)oxy)acetic acid (93 mg, 0.28 mmol), EDCI (80 mg, 0.46 mmol), HOBT (55 mg, 0.46 mmol) and DMAP (56 mg, 0.46 mmol) in DMF (20 mL) was stirred at rt for 3 hours. The solution was diluted with water and extracted with EtOAc (100 mL×2). The combined organic layers were dried over Na₂SO₄, filtered and concentrated. The residue was purified by prep-HPLC to yield the title compound as a white solid (30 mg, 27%). LCMS (ESI): m/z=656.6 (M+H)⁺; RT=1.64 min, purity=98%. ¹H NMR (400 MHz, DMSO) δ 11.14 (s, 1H), 9.69 (s, 1H), 7.96 (s, 1H), 7.80 (t, J=7.8 Hz, 1H), 7.70 (dd, J=8.5, 4.2 Hz, 3H), 7.49 (d, J=7.2 Hz, 1H), 7.39 (d, J=8.5 Hz, 1H), 7.31 (d, J=2.0 Hz, 1H), 6.90 (d, J=8.6 Hz, 1H), 6.64 (d, J=8.6 Hz, 2H), 6.32 (s, 1H), 5.12 (dd, J=12.9, 5.2 Hz, 1H), 4.78 (s, 2H), 3.11 (dd, J=40.7, 5.6 Hz, 4H), 2.96-2.73 (m, 1H), 2.56 (dd, J=20.3, 11.8 Hz, 2H), 2.05 (s, 1H), 1.65-1.30 (m, 8H).

### Example 7: Synthesis of (E)-4-(4-methoxystyryl)-N-methylaniline (control B)

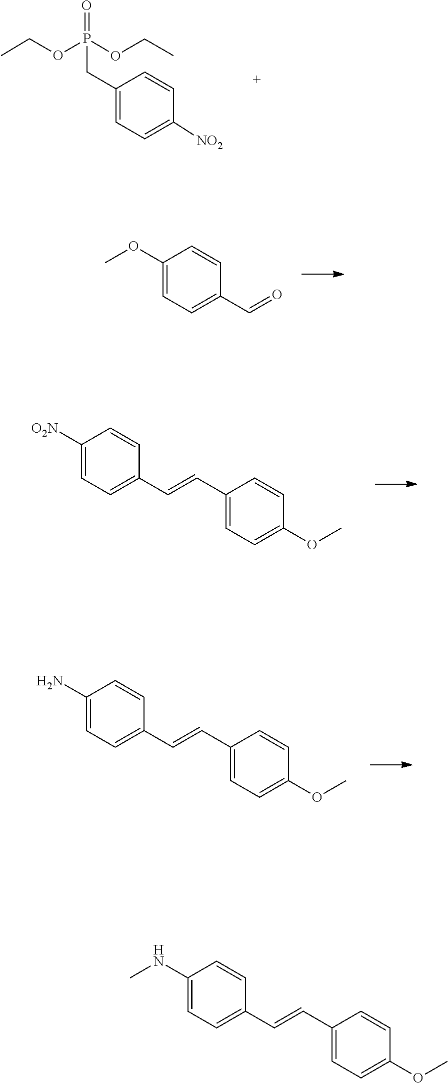

NaOMe (3.24 g, 60 mmol) and 18-Crown-6 (3.17 g, 12 mmol) were added to a solution of diethyl (4-nitrobenzyl)phosphonate (8.0 g, 30 mmol) in DMF (60 mL). The reaction mixture was stirred at rt for 5 minutes and a solution of 4-methoxybenzaldehyde (4.9 g, 36 mmol) in DMF (25 mL) was added dropwise at 0° C. The mixture was stirred at rt for 1 hour and then at 120° C. for 20 hours. The mixture was quenched with water and the precipitate was filtered and washed with water to give (E)-1-methoxy-4-(4-nitrostyryl)benzene as a yellow solid (3.0 g, 39%).

Fe (6.6 g, 118 mmol) was added to a mixture of (E)-1-methoxy-4-(4-nitrostyryl)benzene (3.0 g, 11.8 mmol) in sat. NH₄C₁ solution (40 mL) and MeOH (100 mL) at rt. The reaction mixture was stirred at reflux for 3 hours. The mixture was filtered through a pad of Celite® and washed with MeOH. The filtrate was concentrated in vacuo and diluted with water. The mixture was basified with sat. NaHCO₃ solution to pH=8 and extracted with EtOAc (100 mL×2). The combined organic layers were dried over Na₂SO₄, filtered and concentrated in vacuo to give (E)-4-(4-methoxystyryl)aniline as a light red solid (1.6 g, 60%).

NaOMe (335 mg, 6.2 mmol) was added to a mixture of (E)-4-(4-methoxystyryl)aniline (200 mg, 0.89 mmol) and (CHO). (214 mg, 7.1 mmol) in MeOH (20 mL) at rt. The reaction mixture was stirred at reflux for 3 hours under N₂. The mixture was cooled to 0° C. and NaBH₄ (260 mg, 7.1 mmol) was added. The reaction mixture was stirred at reflux for 1 hour and then diluted with water. The mixture was extracted with EtOAc (100 mL×2). The combined organic layers were dried over Na₂SO₄, filtered and concentrated. The residue was purified by prep-HPLC to yield the title compound as a white solid (75 mg, 26%). ¹H NMR (400 MHz, DMSO) δ 7.44 (d, J=8.7 Hz, 2H), 7.30 (d, J=8.6 Hz, 2H), 6.96-6.80 (m, 4H), 6.52 (d, J=8.5 Hz, 2H), 5.84 (s, 1H), 3.75 (s, 3H), 2.69 (s, 3H).

### Example 8: Synthesis of (E)-2-((2-(2,6-dioxopiperidin-3-yl)-1,3-dioxoisoindolin-4-yl)oxy)-N-(2-(2-(2-(4-(4-(methylamino)styryl)phenoxy)ethoxy)ethoxy)ethyl)acetamide (12)

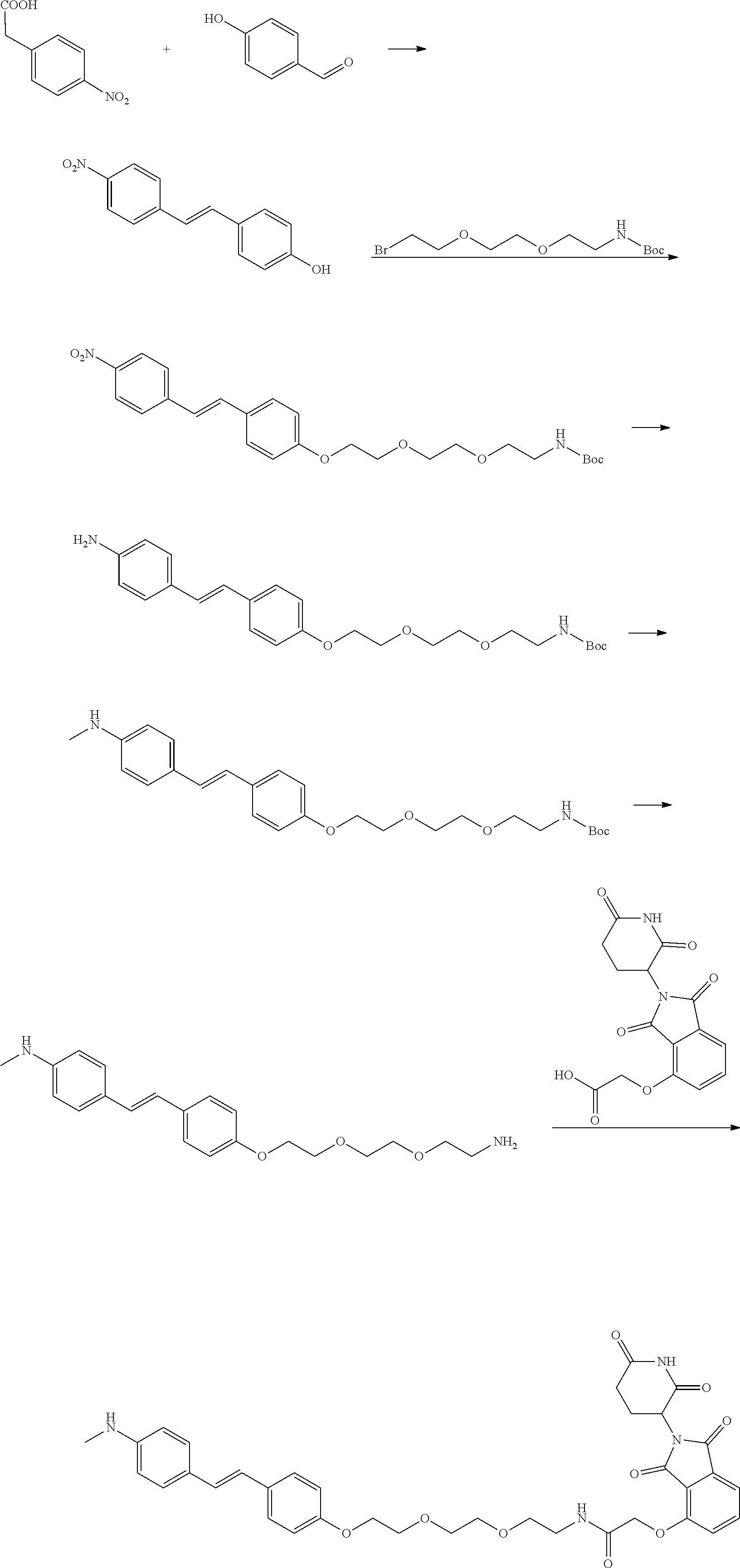

A mixture of 2-(4-nitrophenyl)acetic acid (18 g, 100 mmol) and 4-hydroxybenzaldehyde (24 g, 200 mmol) in piperidine (6 mL) was stirred at 140° C. for 2 hours. The solid was washed with EtOAc to give (E)-4-(4-nitrostyryl)phenol as a red solid (23.6 g, 98%).

A mixture of (E)-4-(4-nitrostyryl)phenol (1.0 g, 4.17 mmol), tert-butyl (2-(2-(2-bromoethoxy)ethoxy)ethyl)carbamate (1.3 g, 4.17 mmol) and K₂CO₃ (1.15 g, 8.34 mmol) in DMF (100 mL) was stirred at 120° C. for 3 hours. The mixture was diluted with water and extracted with EtOAc (100 mL×2). The combined organic layers were dried over Na₂SO₄, filtered and concentrated. The residue was purified through a silica gel column (eluted with EtOAc/PE=2/3) to give Int-11 as a white solid (950 mg, 48%).

Fe (1.13 g, 20.0 mmol) was added to a mixture of tert-butyl (E)-(2-(2-(2-(4-(4-nitrostyryl)phenoxy)ethoxy)ethoxy)ethyl)carbamate (950 mg, 2.0 mmol) in sat. NH₄Cl solution (40 mL) and MeOH (100 mL) at rt. The reaction mixture was stirred at reflux for 3 hours. The mixture was filtered through a pad of Celite® and washed with MeOH. The filtrate was concentrated in vacuo and diluted with water. The mixture was basified with sat. NaHCO₃ solution to pH=8 and extracted with EtOAc (100 mL×2). The combined organic layers were dried over Na₂SO₄, filtered and concentrated in vacuo to give tert-butyl (E)-(2-(2-(2-(4-(4-aminostyryl)phenoxy)ethoxy)ethoxy)ethyl)carbamate as a light red solid (880 mg, 98%).

NaOMe (750 mg, 13.9 mmol) was added to a mixture of tert-butyl (E)-(2-(2-(2-(4-(4-aminostyryl)phenoxy)ethoxy)ethoxy)ethyl)carbamate (880 mg, 1.99 mmol) and (CHO)ₙ (477 mg, 15.9 mmol) in MeOH (100 mL) at rt. The reaction mixture was stirred at reflux for 3 hours under N₂. The mixture was cooled to 0° C. and NaBH₄ (605 mg, 15.9 mmol) was added. The reaction mixture was stirred at reflux for 1 hour and diluted with water. The mixture was extracted with EtOAc (100 mL×2). The combined organic layers were dried over Na₂SO₄, filtered and concentrated. The residue was purified through a silica gel column (eluted with EtOAc/PE=1/2) to give tert-butyl (E)-(2-(2-(2-(4-(4-(methylamino)styryl)phenoxy)ethoxy)ethoxy)ethyl)carbamate as a white solid (500 mg, 50%).

HCl in dioxane (10 mL) was added to a solution of tert-butyl (E)-(2-(2-(2-(4-(4-(methylamino)styryl)phenoxy)ethoxy)ethoxy)ethyl)carbamate (500 mg, 1.1 mmol) in DCM (10 mL) at rt. The reaction mixture was stirred at rt for 3 hours. The mixture was concentrated in vacuo, diluted with water and basified to pH=8 with sat. NaHCO₃. The precipitate was filtered off and washed with water to give (E)-4-(4-(2-(2-(2-aminoethoxy)ethoxy)ethoxy)styryl)-N-methylaniline as a white solid (390 mg, 100%).

A mixture of (E)-4-(4-(2-(2-(2-aminoethoxy)ethoxy)ethoxy)styryl)-N-methylaniline (200 mg, 0.56 mmol), 2-((2-(2,6-dioxopiperidin-3-yl)-1,3-dioxoisoindolin-4-yl)oxy)acetic acid (186 mg, 0.56 mmol), EDCI (161 mg, 0.84 mmol), HOBT (113 mg, 0.84 mmol) and DMAP (137 mg, 1.12 mmol) in DMF (20 mL) was stirred at rt for 3 hours. The solution was diluted with water and extracted with EtOAc (100 mL×2). The combined organic layers were dried over Na₂SO₄, filtered and concentrated. The residue was purified by prep-HPLC to yield the title compound as a white solid (100 mg, 27%). ¹H NMR (400 MHz, DMSO) δ 11.14 (s, 1H), 8.02 (s, 1H), 7.86-7.73 (m, 1H), 7.49 (d, J=7.3 Hz, 1H), 7.40 (t, J=7.9 Hz, 3H), 7.30 (d, J=8.6 Hz, 2H), 6.95-6.78 (m, 4H), 6.52 (d, J=8.6 Hz, 2H), 5.83 (d, J=4.9 Hz, 1H), 5.12 (dd, J=13.0, 5.3 Hz, 1H), 4.78 (s, 2H), 4.15-4.03 (m, 2H), 3.82-3.68 (m, 2H), 3.62-3.53 (m, 4H), 3.48 (t, J=5.6 Hz, 2H), 2.99-2.79 (m, 1H), 2.69 (d, J=4.8 Hz, 3H), 2.64-2.52 (m, 4H), 2.04 (s, 1H).

### Example 9: Synthesis of (E)-6-(4-(4-(methylamino)styryl)phenoxy)hexyl 2-((2-(2,6-dioxopiperidin-3-yl)-1,3-dioxoisoindolin-4-yl)oxy)acetate (13)

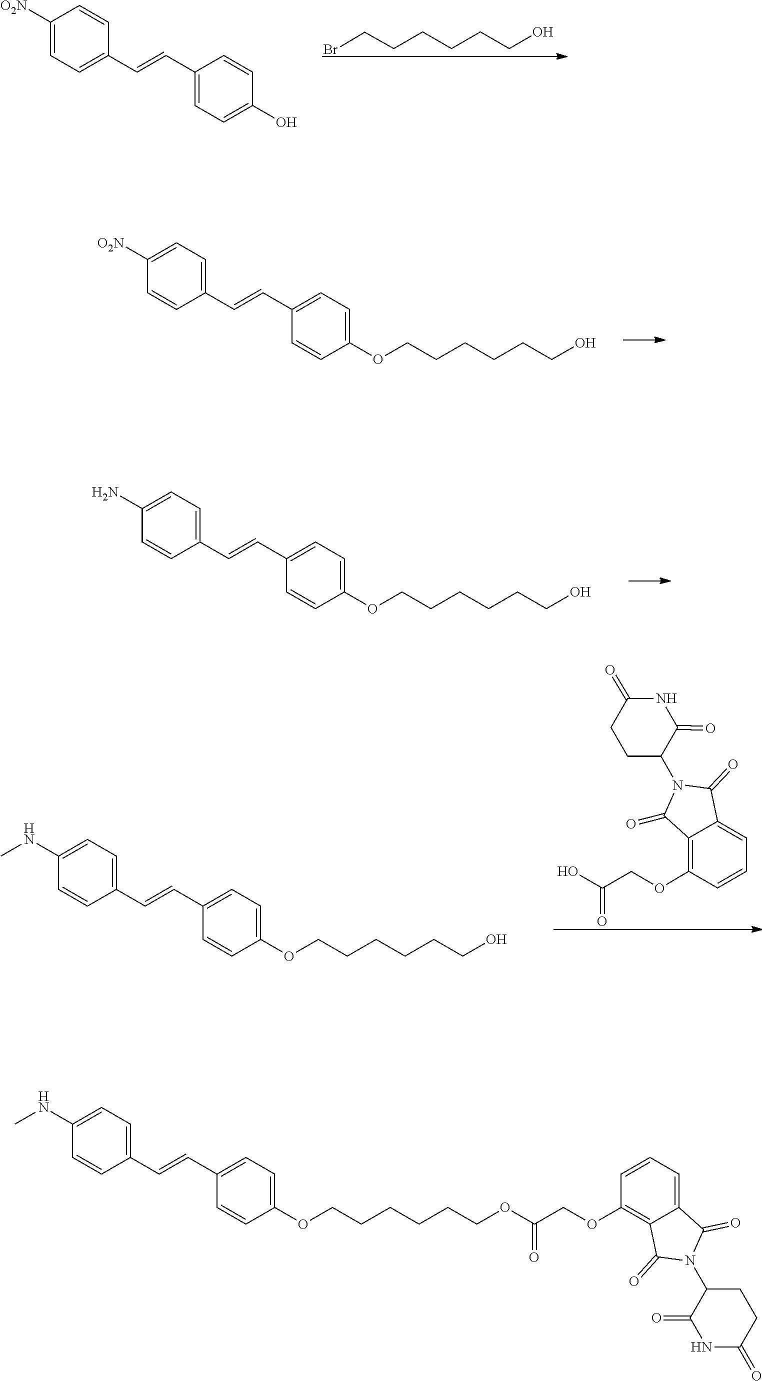

A mixture of (E)-4-(4-nitrostyryl)phenol (6.0 g, 24.9 mmol), 6-bromohexan-1-ol (4.48 g, 24.9 mmol) and K₂CO₃ (10.4 g, 75 mmol) in CH₃CN (300 mL) was stirred at 80° C. for 20 hours. The mixture was cooled to rt and the precipitate was filtered off and washed with EtOAc to give (E)-6-(4-(4-nitrostyryl)phenoxy)hexan-1-ol as a light yellow solid (5.0 g, 59%).

Fe (8.2 g, 147 mmol) was added to a mixture of (E)-6-(4-(4-nitrostyryl)phenoxy)hexan-1-ol (5.0 g, 14.7 mmol) in sat. NH₄Cl solution (40 mL) and MeOH (200 mL) at rt. The reaction mixture was stirred at reflux for 3 hours. The mixture was filtered through a pad of Celite® and washed with MeOH. The filtrate was concentrated in vacuo and diluted with water. The mixture was basified with sat. NaHCO₃ solution to pH=8 and extracted with EtOAc (100 mL×2). The combined organic layers were dried over Na₂SO₄, filtered and concentrated in vacuo to give (E)-6-(4-(4-aminostyryl)phenoxy)hexan-1-ol as a light red solid (3.2 g, 70%).

NaOMe (3.9 g, 72.1 mmol) was added to a mixture of (E)-6-(4-(4-aminostyryl)phenoxy)hexan-1-ol (3.2 g, 10.3 mmol) and (CHO)ₙ(2.47 g, 82.3 mmol) in MeOH (200 mL) at rt. The reaction mixture was stirred at reflux for 3 hours under N₂. The mixture was cooled to 0° C. and NaBH₄ (3.13 g, 82.3 mmol) was added. The reaction mixture was stirred at reflux for 1 hour and diluted with water. The mixture was extracted with EtOAc (100 mL×2). The combined organic layers were dried over Na₂SO₄, filtered and concentrated. The residue was purified through a silica gel column (elute with EtOAc/PE=1/2) to give (E)-6-(4-(4-(methylamino)styryl)phenoxy)hexan-1-ol as a white solid (3.0 g, 90%).

A mixture of (E)-6-(4-(4-(methylamino)styryl)phenoxy)hexan-1-ol (182 mg, 0.56 mmol), 2-((2-(2,6-dioxopiperidin-3-yl)-1,3-dioxoisoindolin-4-yl)oxy)acetic acid (186 mg, 0.56 mmol), EDCI (161 mg, 0.84 mmol), HOBT (113 mg, 0.84 mmol) and DMAP (137 mg, 1.12 mmol) in DMF (20 mL) was stirred at rt for 3 hours. The solution was diluted with water and extracted with EtOAc (100 mL×2). The combined organic layers were dried over Na₂SO₄, filtered and concentrated. The residue was purified by prep-HPLC to yield the title compound as a white solid (90 mg, 25%). ¹H NMR (400 MHz, DMSO) δ 11.13 (s, 1H), 7.79 (dd, J=8.4, 7.4 Hz, 1H), 7.49 (d, J=7.2 Hz, 1H), 7.42 (d, J=8.7 Hz, 3H), 7.30 (d, J=8.6 Hz, 2H), 6.98-6.78 (m, 4H), 6.52 (d, J=8.6 Hz, 2H), 5.87 (s, 1H), 5.21-5.05 (m, 3H), 4.15 (t, J=6.5 Hz, 2H), 3.94 (t, J=6.4 Hz, 2H), 2.97-2.80 (m, 1H), 2.69 (s, 3H), 2.62-2.52 (m, 2H), 2.11-1.96 (m, 1H), 1.66 (ddd, J=22.0, 21.4, 15.0 Hz, 4H), 1.47-1.31 (m, 4H).

### Example 10: Synthesis of (E)-1-(4-methoxybenzyl-3-((E)-3-(4-nitrophenyl)allylidene)indolin-2-one (control A)

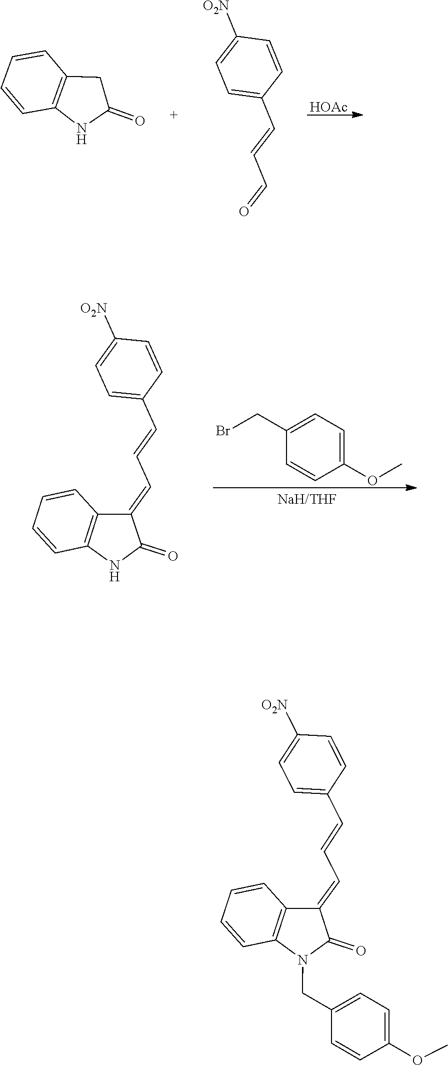

A solution of 2-oxindole (2.64 g, 20 mmol) and 3-(4-nitrophenyl)prop-2-enal (3.53 g, 20 mmol) in acetic acid (50 mL) and 37% HCl (1 mL) was stirred at reflux for 3 hours. The reaction was cooled to rt and water (500 mL) was added. The solids were filtered and recrystallized in methanol to yield Int-18 as a red solid (3 g, 51%). LCMS (ESI): m/z=293.1 (M+H)⁺; RT=1.83 min.

A solution of (E)-3-((E)-3-(4-nitrophenyl)allylidene)indolin-2-one (1.0 mmol) in THF (5 mL) was added to 60% NaH (1.5 mmol) at 0° C. After 15 minutes, 4-methoxybenzyl bromide (3 mmol) was added and stirred for 8 hours. The reaction mixture was diluted with EtOAc (75 mL), and the organic layer was washed with water (50 mL×2) and saturated NaCl (50 mL), and dried over Na₂SO₄. The crude was concentrated in vacuo and purified by silica gel column chromatography eluting with hexanes:DCM:EtOAc (10:10:3, v/v/v) to yield the title compound. ¹H NMR (400 MHz, CDCl3) δ 8.28 (d, J=8.6 Hz, 2H), 7.84-7.68 (m, 4H), 7.53 (d, J=12.1 Hz, 1H), 7.27 (s, 1H), 7.21 (dd, J=18.1, 13.4 Hz, 3H), 7.07 (t, J=7.6 Hz, 1H), 6.85 (d, J=8.5 Hz, 2H), 6.78 (d, J=7.9 Hz, 1H), 4.92 (s, 2H), 3.77 (s, 3H). LCMS (ESI): m/z=413.2 (M+H)⁺; RT=2.04 min.

### Example 11: Synthesis of 3-(2-(2-(2-((2-(2,6-dioxopiperidin-3-yl)-1,3-dioxoisoindolin-4-yl)oxy)ethoxy)ethoxy)ethoxy)-N-(2-(4-(((E)-3-((E)-3-(4-nitrophenyl)allylidene)-2-oxoindolin-1-yl)methyl)phenoxy)ethyl)propanamide (1)

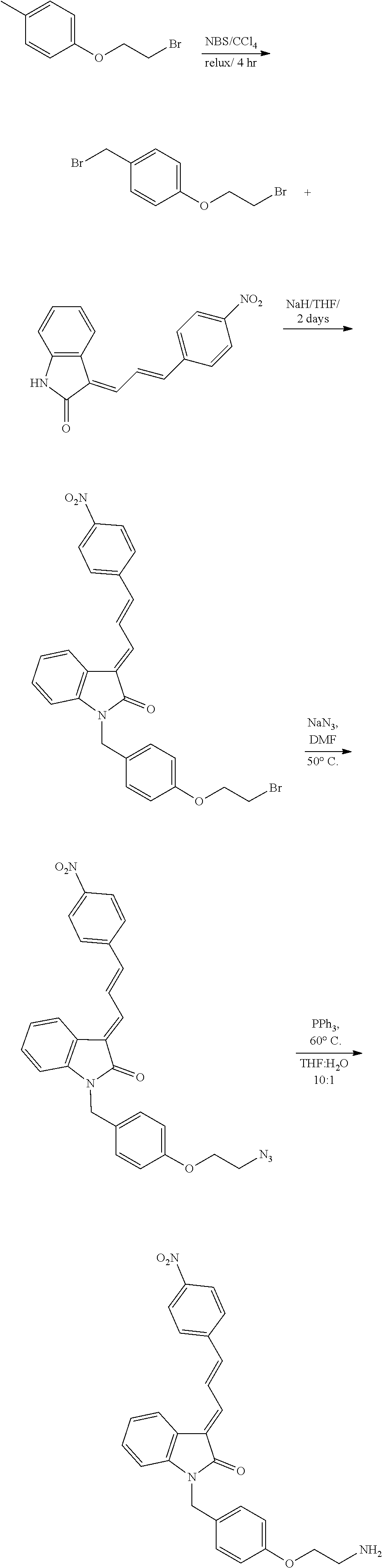 [SMILES: Cc1ccc(OCCBr)cc1 ; BrCCOc1ccc(CBr)cc1 ; O=C1Nc2ccccc2/C1=C\C=C\c1ccc([N+](=O)[O-])cc1 ; O=C1/C(=C/C=C/c2ccc([N+](=O)[O-])cc2)c2ccccc2N1Cc1ccc(OCCBr)cc1 ; [N-]=[N+]=NCCOc1ccc(CN2C(=O)/C(=C/C=C/c3ccc([N+](=O)[O-])cc3)c3ccccc32)cc1 ; NCCOc1ccc(CN2C(=O)/C(=C/C=C/c3ccc([N+](=O)[O-])cc3)c3ccccc32)cc1]

p-(2-Bromoethoxy)toluene (10 g, 0.046 mol), NBS (12.28 g, 0.069 mol) and AIBN (1.0 g) were dissolved in CCl₄ (50 ml) and heated to reflux for 3.5 hours. After cooling to rt, the reaction mixture was washed with saturated NaHCO₃ and brine, dried with magnesium sulfate and concentrated in vacuo. Immediate filtration through silica gel (petroleum ether:EtOAc=9:1) yielded 1-(2-bromoethoxy)-4-(bromomethyl)benzene as a white solid (8 g, 59.2%).

60% NaH (0.36 g, 27 mmol) was added to a solution of (E)-3-((E)-3-(4-nitrophenyl)allylidene)indolin-2-one (2.6 g, 9 mmol) in THF (50 mL) was added at 0° C. After 15 minutes, 1-(2-bromoethoxy)-4-(bromomethyl)benzene (8 g, 27 mmol) was added to the reaction mixture and stirred for 48 hours. EtOAc (500 mL) was added, and washed with water (50 mL×2) and saturated NaCl (50 mL), and dried over Na₂SO₄. The organic layer was concentrated in vacuo and purified by silica gel column chromatography eluting with PE/EtOAc (10:3, v/v) to afford (E)-1-(4-(2-bromoethoxy)benzyl)-3-((E)-3-(4-nitrophenyl)allylidene)indolin-2-one as a white solid (1.5 g, 33%).

A mixture of (E)-1-(4-(2-bromoethoxy)benzyl)-3-((E)-3-(4-nitrophenyl)allylidene)indolin-2-one (1.5 g, 29 mmol) and NaN₃ (0.4 g, 58 mmol) in DMF (20 mL) was stirred at 50° C. for 2 hours. The reaction mixture was portioned between EtOAc (50 mL) and water (50 mL). The aqueous layers were extracted with EtOAc (50 mL×2), and the combined organic layers were dried over anhydrous Na₂SO₄, filtered and concentrated. The residue was purified by silica gel (PE:EtOAc=5:1) to yield (E)-1-(4-(2-azidoethoxy)benzyl)-3-((E)-3-(4-nitrophenyl)allylidene)indolin-2-one as a yellow solid (1.1 g, 80%).

The mixture of (E)-1-(4-(2-azidoethoxy)benzyl)-3-((E)-3-(4-nitrophenyl)allylidene)indolin-2-one (1.1 g, 24 mmol) and PPh₃ (1.23 g, 47 mmol) in THF:H₂O (10:1, 30 mL) was stirred at 50° C. for 24 hours. The aqueous layer was extracted with EtOAc (50 mL×2), and the combined organic layers were dried over Na₂SO₄. The organic layer was concentrated in vacuo and purified by silica gel column chromatography (PE:EtOAc=5:1) to yield (E)-1-(4-(2-aminoethoxy)benzyl)-3-((E)-3-(4-nitrophenyl)allylidene)indolin-2-one as a white solid (500 mg, 47%).

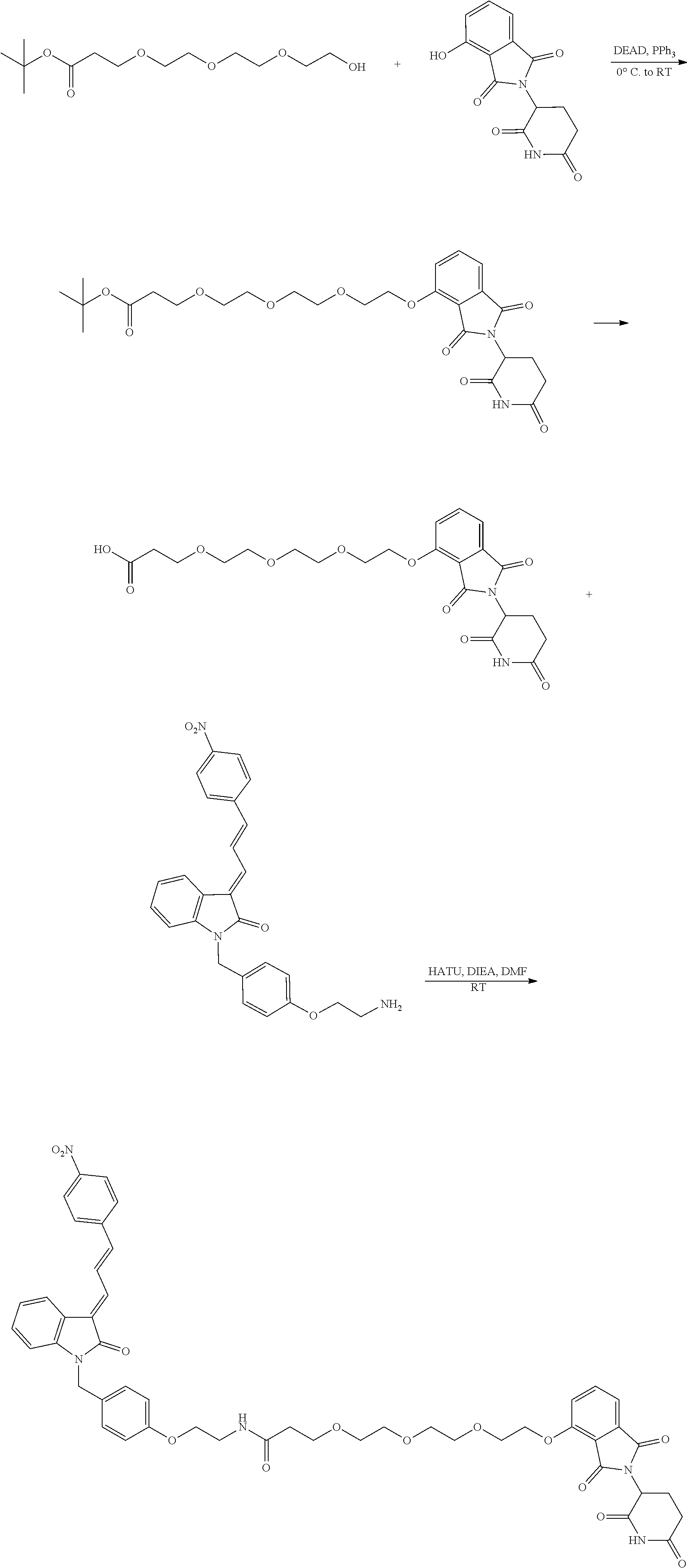

Diethyl azodicarboxylate (0.62 g, 36 mmol) was added to a mixture of 2-(2,6-dioxopiperidin-3-yl)-4-hydroxyisoindole-1,3-dione (0.54 g, 19.8 mmol), tert-butyl 12-hydroxy-4,7,10-trioxadodecanoate (0.5 g, 18 mmol) and PPh₃ (0.94 g, 36 mmol) in DCM (20 mL) at 0° C. The reaction mixture was stirred at rt for 8 hours. The reaction was quenched with water (100 mL) and extracted with DCM (50 mL×2), and dried over Na₂SO₄. The organic layer was concentrated in vacuo and purified by silica gel column chromatography (PE:EtOAc=4:1) to yield tert-butyl 3-(2-(2-(2-((2-(2,6-dioxopiperidin-3-yl)-1,3-dioxoisoindolin-4-yl)oxy)ethoxy)ethoxy)ethoxy)propanoate as a white solid (300 mg, 31%).

A mixture of tert-butyl 3-(2-(2-(2-((2-(2,6-dioxopiperidin-3-yl)-1,3-dioxoisoindolin-4-yl)oxy)ethoxy)ethoxy)ethoxy)propanoate (0.3 g, 0.56 mmol) in DCM:TFA (10:2, 5 mL) was stirred at rt for 1 hour. The reaction mixture was concentrated in vacuo and purified by silica gel column chromatography (DCM:MeOH=20:1) to yield 3-(2-(2-(2-((2-(2,6-dioxopiperidin-3-yl)-1,3-dioxoisoindolin-4-yl)oxy)ethoxy)ethoxy)ethoxy)propanoic acid as a white solid (150 mg, 56%).

A mixture of 3-(2-(2-(2-((2-(2,6-dioxopiperidin-3-yl)-1,3-dioxoisoindolin-4-yl)oxy)ethoxy)ethoxy)ethoxy)propanoic acid (150 mg, 0.3 mmol), (E)-1-(4-(2-aminoethoxy)benzyl)-3-((E)-3-(4-nitrophenyl)allylidene)indolin-2-one (110 mg, 0.25 mmol), HATU (285 mg 0.75 mmol) and DIEA (96 mg, 0.75 mmol) in DMF (10 ml) was stirred at rt for 3 hours. The mixture was portioned between EtOAc (50 mL) and water (50 mL). The aqueous layers were extracted with EtOAc (50 mL×2), and the combined organic layers were dried over anhydrous Na₂SO₄, filtered and concentrated. The residue was purified by prep-HPLC to yield the title compound as a red solid (8 mg, 3.5%). ¹H NMR (400 MHz, DMSO) δ 11.12 (s, 1H), 8.70 (dd, 1H), 8.29 (d, J=8.9 Hz, 2H), 8.08 (s, 1H), 7.86 (d, 2H), 7.82-7.61 (m, 3H), 7.50 (d, 1H), 7.44 (d, 1H), 7.37-7.10 (m, 4H), 7.02 (t, 1H), 6.97 (d, 1H), 6.89 (d, 2H), 6.55 (s, 1H), 5.08 (dd, 1H), 4.88 (s, 2H), 4.37-4.20 (m, 2H), 3.91 (t, 2H), 3.82-3.71 (m, 2H), 3.57 (dt, 2H), 3.48-3.42 (m, 4H), 3.40-3.35 (m, 2H), 3.32 (s, 2H), 3.03-2.76 (m, 2H), 2.70-2.54 (m, 2H), 2.30 (t, 2H), 2.08-1.86 (m, 2H). LCMS (ESI): m/z 902.0 (M+H)⁺; RT=1.81 min.

### Example 12: Synthesis of 7-((2-(2,6-dioxopiperidin-3-yl)-1,3-dioxoisoindolin-4-yl)oxy)-N-(2-(4-(((E)-3-((E)-3-(4-nitrophenyl)allylidene)-2-oxoindolin-1-yl)methyl)phenoxy)ethyl)heptanamide (2)

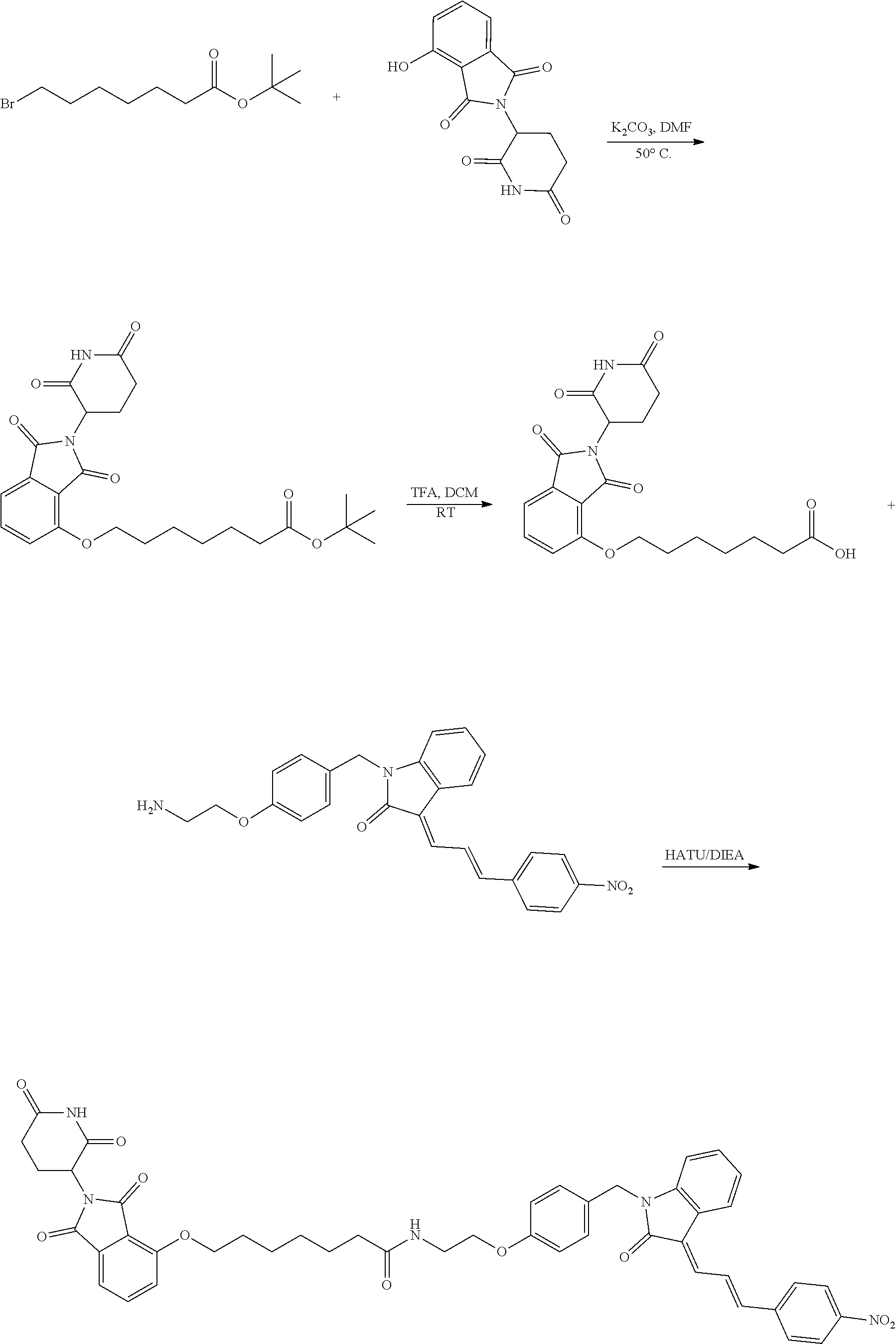

A mixture of tert-butyl 7-bromoheptanoate (0.5 g, 1.8 mol), 2-(2,6-dioxopiperidin-3-yl)-4-hydroxyisoindole-1,3-dione (0.6 g, 2.2 mmol) and K₂C₃ (0.745 g, 5.4 mmol) in DMF (20 mL) was stirred at 50° C. for 3 hours. The mixture was treated with water, and extracted with EtOAc (50 mL×2), and dried over Na₂SO₄. The organic layer was concentrated in vacuo and purified by CombiFlash® (PE:EtOAc=5:1) to yield tert-butyl 7-((2-(2,6-dioxopiperidin-3-yl)-1,3-dioxoisoindolin-4-yl)oxy)heptanoate as a white solid (300 mg, 31%).

A solution of tert-butyl 7-((2-(2,6-dioxopiperidin-3-yl)-1,3-dioxoisoindolin-4-yl)oxy)heptanoate (0.3 g, 0.56 mmol) in DCM:TFA (10:2, 5 mL) was stirred at rt for 1 hour. The reaction mixture was concentrated in vacuo and purified by silica gel column chromatography (DCM:MeOH=20:1) to yield 7-((2-(2,6-dioxopiperidin-3-yl)-1,3-dioxoisoindolin-4-yl)oxy)heptanoic acid as a white solid (200 mg, 75%).

A mixture of 7-((2-(2,6-dioxopiperidin-3-yl)-1,3-dioxoisoindolin-4-yl)oxy)heptanoic acid (200 mg, 0.5 mmol), (E)-1-(4-(2-aminoethoxy)benzyl)-3-((E)-3-(4-nitrophenyl)allylidene)indolin-2-one (360 mg, 0.5 mmol), HATU (570 mg 1.5 mmol) and DIEA (200 mg, 1.5 mmol) in DMF (30 mL) was stirred at rt for 3 hours. The mixture was portioned between EtOAc (150 mL) and water (150 mL). The aqueous layer was extracted with EtOAc (150 mL×2). The combined organic layers were dried over anhydrous Na₂SO₄, filtered and concentrated. The residue was purified by prep-HPLC to yield the title compound as a red solid (25 mg, 6%). ¹H NMR (400 MHz, DMSO) δ 11.12 (s, 1H), 8.27 (d, J=8.8 Hz, 2H), 8.10 (dd, J=12.1, 8.3 Hz, 3H), 8.01 (dd, J=10.7, 4.8 Hz, 1H), 7.98-7.91 (m, 1H), 7.83-7.74 (m, 1H), 7.56 (d, J=15.3 Hz, 1H), 7.52-7.40 (m, 3H), 7.27 (t, J=8.2 Hz, 3H), 7.07 (t, J=7.5 Hz, 1H), 6.98 (d, J=7.9 Hz, 1H), 6.89 (d, J=8.6 Hz, 2H), 5.08 (dd, J=13.0, 5.2 Hz, 1H), 4.88 (s, 2H), 4.13 (t, J=6.1 Hz, 2H), 3.92 (t, J=5.5 Hz, 2H), 3.37 (s, 1H), 3.32 (s, 1H), 2.86 (d, J=11.6 Hz, 1H), 2.70 (d, J=24.1 Hz, 1H), 2.07 (t, J=7.2 Hz, 2H), 1.74-1.60 (m, 2H), 1.54-1.45 (m, 2H), 1.41 (s, 2H), 1.27 (d, J=6.9 Hz, 2H). LCMS (ESI): m/z 826.0 (M+H)⁺; RT=1.98 min.

### Example 13: Cellular CRBN Target Engagement Data

HEK293T cells stably expressing the BRD4_{BD2}-GFP with mCherry reporter were seeded at 30-50% confluency in 384-well plates with 50 μL FluoroBrite™ DMEM media (Thermo Fisher Scientific A18967) containing 10% FBS per well a day before compound treatment. Degrader titrations and 100 nM dBET6,

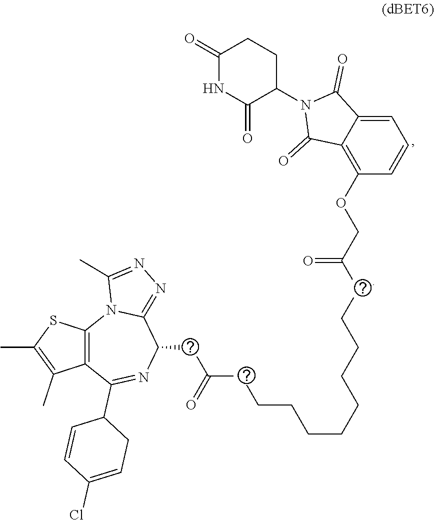 [SMILES: Cc1sc2c(c1C)C(C1C=CC(Cl)=CC1)=N[C@@H](OC(=O)NCCCCCCCCNC(=O)COc1cccc3c1C(=O)N(C1CCC(=O)NC1=O)C3=O)c1nnc(C)n1-2]

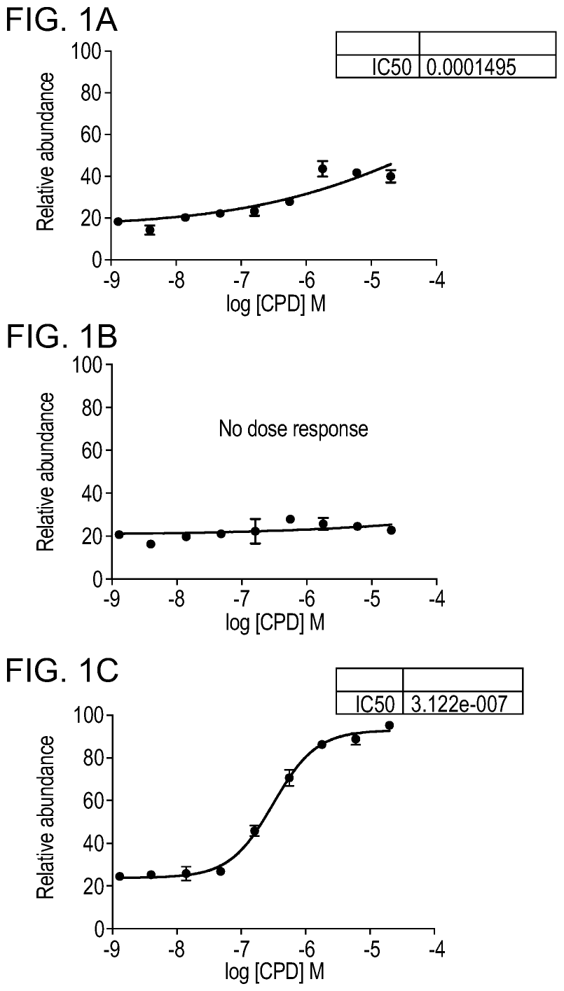

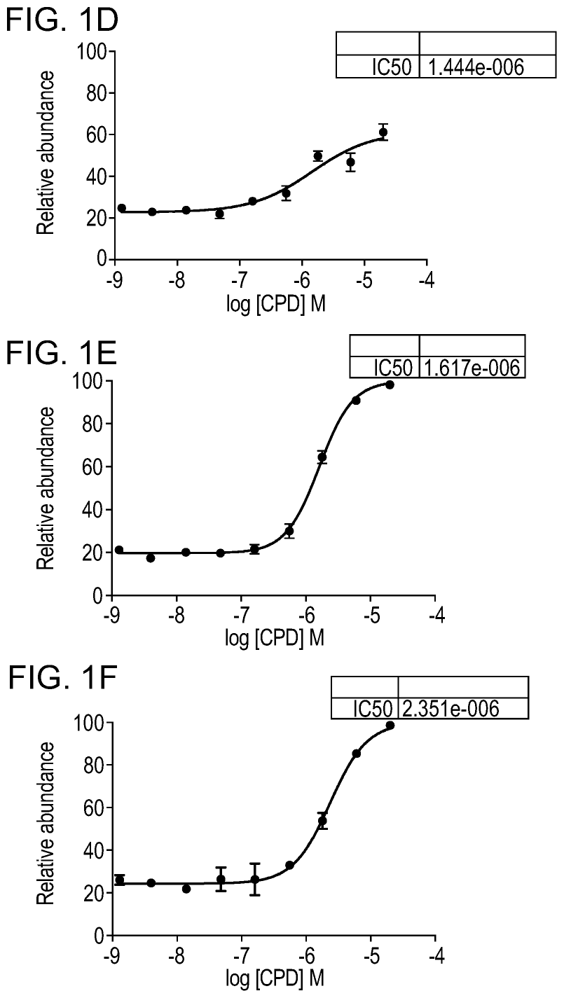

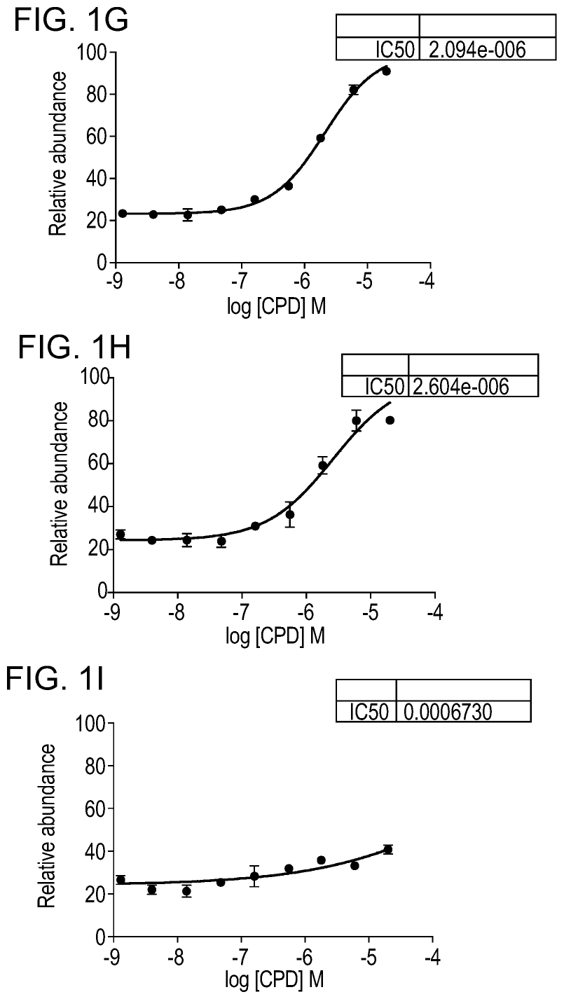

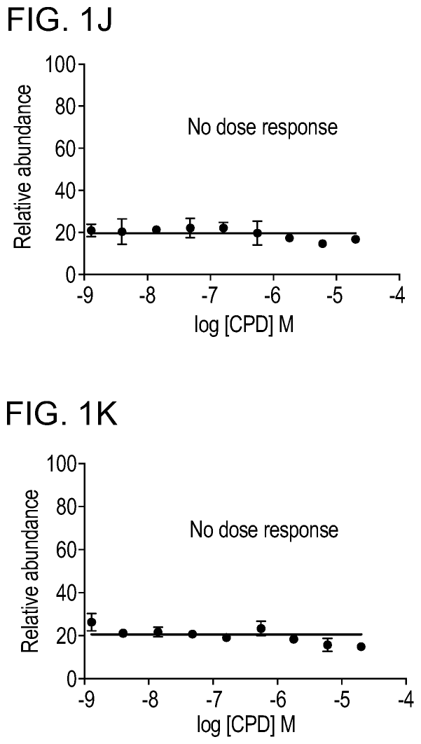

were dispensed using a D300e Digital Dispenser (HP), normalized to 0.5% DMSO, and incubated with cells for 5 hours. Assay plates were imaged using Acumen (ITP Labtech). Experiments were performed in triplicates and the values for the concentrations that lead to a 50% increase in BRD4_{BD2}-eGFP accumulation (IC₅₀) were calculated using the nonlinear fit variable slope model (GraphPad Software). dBET6 is a known binder of CRBN. The results of the assay indicated that inventive compounds competed with dBET6 for cellular CRBN binding (FIG. 1A-FIG. 1K). These data show that the compounds can permeate the cell and engage the E3-ligase CRBN, which is a requirement for targeted protein degradation.

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A bispecific compound having a structure represented by formula (I): 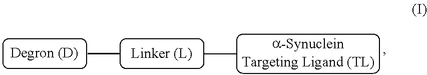
wherein the degron represents a moiety that binds an E3 ubiquitin ligase, and the linker covalently connects the degron and the targeting ligand, or a pharmaceutically acceptable salt or stereoisomer thereof,
wherein 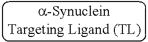
is represented by TL-1, TL-2, TL-3, TL-4, or TL-5: 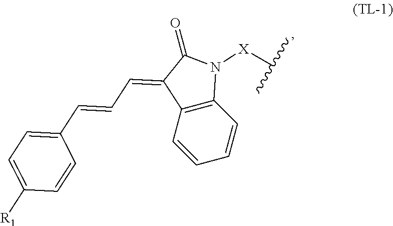
wherein:
X is absent or 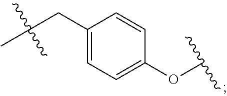 [SMILES: *Cc1ccc(O*)cc1]
and
R₁ is nitro or amino; 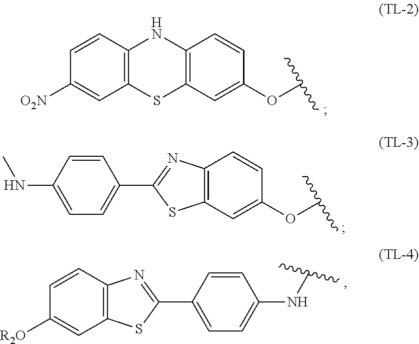
wherein:
R₂ is hydrogen or methyl; or 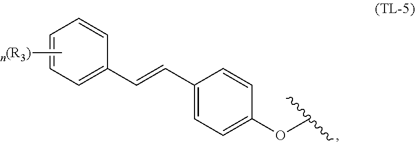
wherein:
R₃ is alkyl, alkenyl, alkynyl, halo, haloalkyl, cycloalkyl, heterocycloalkyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylaminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, azido, phosphinyl, phosphoryl, aryl, or heteroaryl, said R₃ groups may be further optionally substituted; and
n is 0, 1, 2, 3, 4, or 5; and
wherein 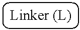
is a polyethylene glycol chain which terminates at either or both termini in —R′C(O)N(R′)R′—, wherein R′ is H or C₁-C₆ alkyl; or
an alkylene chain or a bivalent alkylene chain, either of which may be interrupted by, and/or terminate at either or both termini in —R′C(O)N(R′)R′—, wherein R′ is H or C₁-C₆ alkyl; and
wherein 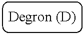
is represented by any one of formulas D1-a to D1-i: 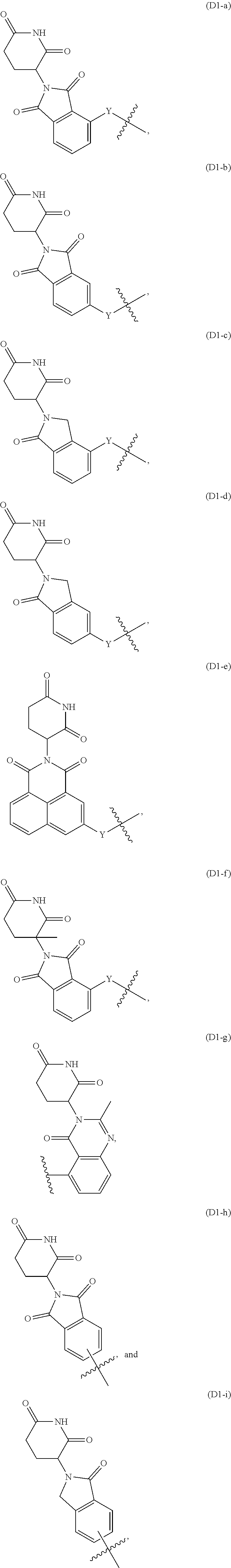
wherein
Y is NH or O,
or a pharmaceutically acceptable salt or stereoisomer thereof.

2. The bispecific compound of claim 1, wherein 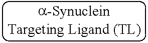
is represented by the formula TL-1:

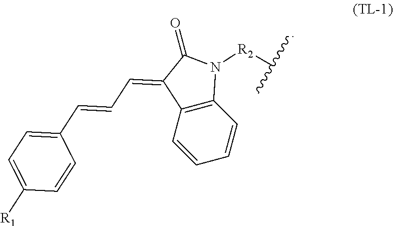

3. The bispecific compound of claim 2, wherein R₁ is NH₂ and R₂ is absent and the bispecific compound is represented by the formula (I-la): 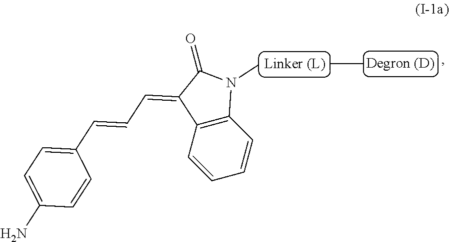
or a pharmaceutically acceptable salt or stereoisomer thereof.

4. The bispecific compound of claim 2, wherein R₁ is NO₂ and R₂ is 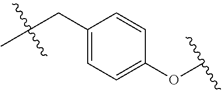 [SMILES: *Cc1ccc(O*)cc1]
and the bispecific compound is represented by the formula (I-1b): 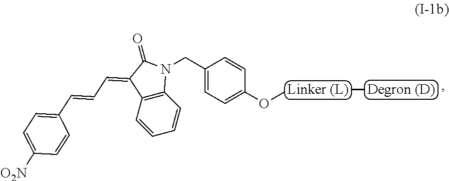
or a pharmaceutically acceptable salt or stereoisomer thereof.

5. The bispecific compound of claim 1, wherein 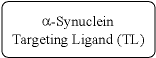
is represented by the formula TL-2:

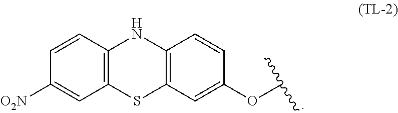 [SMILES: *Oc1ccc2c(c1)Sc1cc([N+](=O)[O-])ccc1N2]

6. The bispecific compound of claim 1, wherein 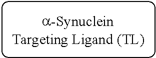
is represented by the formula TL-3:

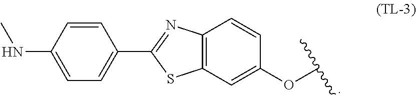 [SMILES: *Oc1ccc2nc(-c3ccc(NC)cc3)sc2c1]

7. The bispecific compound of claim 1, wherein 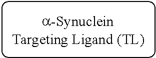
is represented by the formula TL-4:

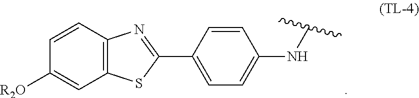

8. The bispecific compound of claim 7, wherein R₂ is hydrogen and bispecific compound is represented b the formula (I-4a): 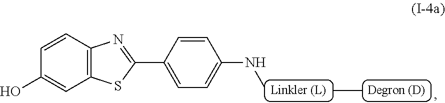
or a pharmaceutically acceptable salt or stereoisomer thereof.

9. The bispecific compound of claim 1, wherein 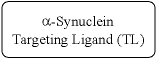
is represented by the formula TL-5:

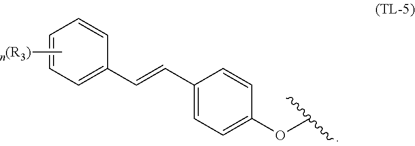

10. The bispecific compound of claim 9, wherein R₃ is 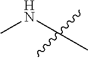 [SMILES: *NC]
and n is 1 and the bispecific compound is represented by the formula (I-5a): 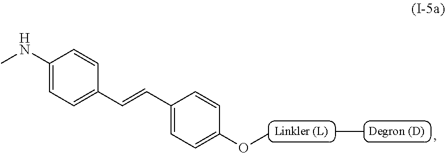
or a pharmaceutically acceptable salt or stereoisomer thereof.

11. The bispecific compound of claim 1, wherein the linker comprises an alkylene chain or a bivalent alkylene chain, either of which may be interrupted by, and/or terminate at either or both termini in —R′C(O)N(R′)R′—, wherein R′ is H or C₁-C₆ alkyl.

12. The bispecific compound of claim 11, wherein the linker comprises an alkylene chain having 1-10 alkylene units and is interrupted by or terminates in

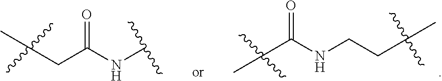 [SMILES: *CC(=O)N* ; *CCNC(*)=O]

13. The bispecific compound of claim 1, wherein the linker comprises a polyethylene glycol chain which terminates at either or both termini in —R′C(O)N(R′)R′—, wherein R′ is H or C₁-C₆ alkyl.

14. The bispecific compound of claim 13, wherein the linker comprises a polyethylene glycol chain having 2-8 PEG units and terminating in

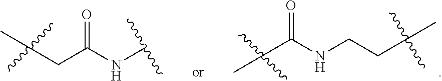 [SMILES: *CC(=O)N* ; *CCNC(*)=O]

15. The bispecific compound of claim 1, which is represented by any one of the following formulas: 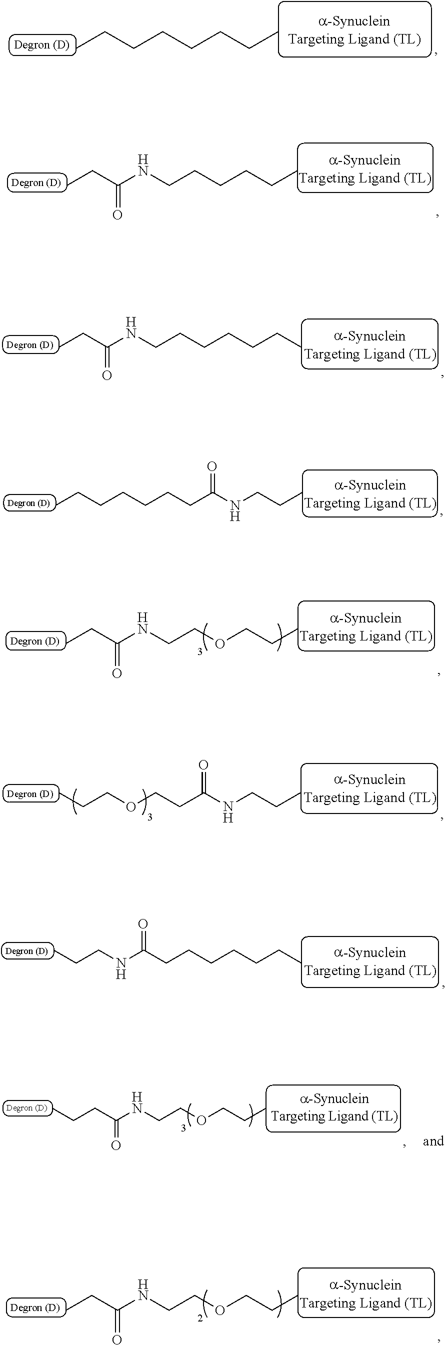
or a pharmaceutically acceptable salt or stereoisomer thereof.

16. The bispecific compound of claim 1, wherein the degron is

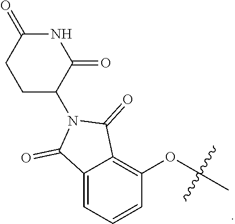 [SMILES: *Oc1cccc2c1C(=O)N(C1CCC(=O)NC1=O)C2=O]

17. The bispecific compound of claim 1, which is: 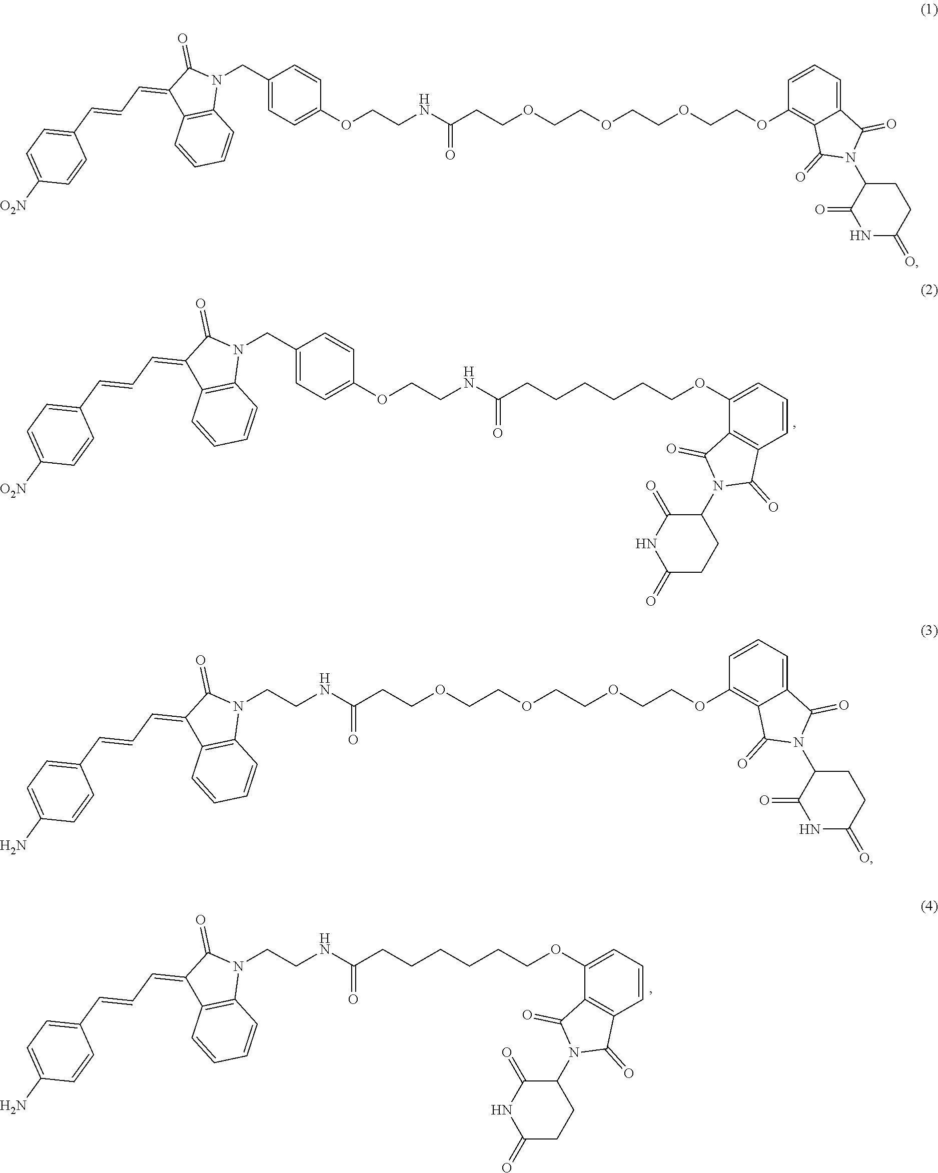 [SMILES: O=C(CCOCCOCCOCCOc1cccc2c1C(=O)N(C1CCC(=O)NC1=O)C2=O)NCCOc1ccc(CN2C(=O)/C(=C/C=C/c3ccc([N+](=O)[O-])cc3)c3ccccc32)cc1 ; O=C(CCCCCCOc1cccc2c1C(=O)N(C1CCC(=O)NC1=O)C2=O)NCCOc1ccc(CN2C(=O)/C(=C/C=C/c3ccc([N+](=O)[O-])cc3)c3ccccc32)cc1 ; Nc1ccc(/C=C/C=C2/C(=O)N(CCNC(=O)CCOCCOCCOCCOc3cccc4c3C(=O)N(C3CCC(=O)NC3=O)C4=O)c3ccccc32)cc1 ; Nc1ccc(/C=C/C=C2/C(=O)N(CCNC(=O)CCCCCCOc3cccc4c3C(=O)N(C3CCC(=O)NC3=O)C4=O)c3ccccc32)cc1] 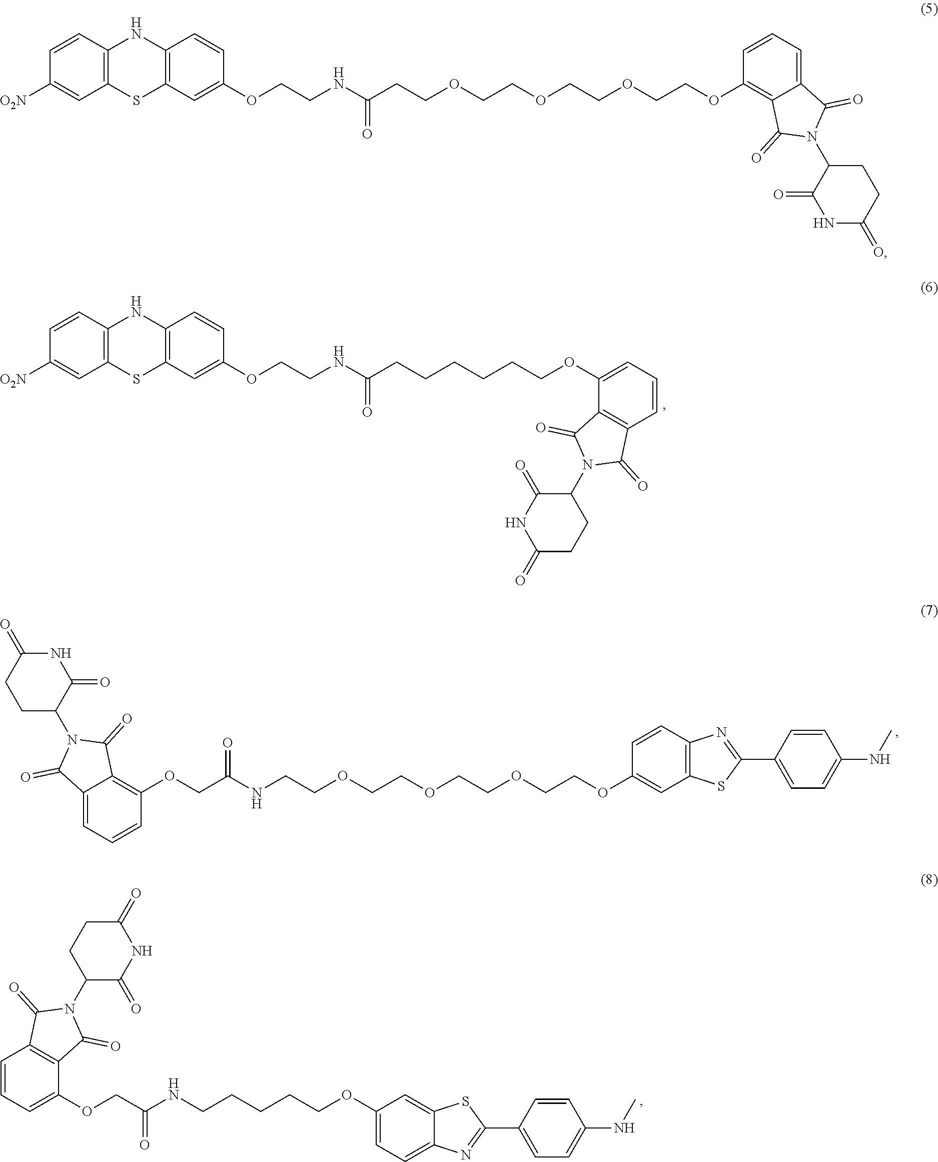 [SMILES: O=C(CCOCCOCCOCCOc1cccc2c1C(=O)N(C1CCC(=O)NC1=O)C2=O)NCCOc1ccc2c(c1)Sc1cc([N+](=O)[O-])ccc1N2 ; O=C(CCCCCCOc1cccc2c1C(=O)N(C1CCC(=O)NC1=O)C2=O)NCCOc1ccc2c(c1)Sc1cc([N+](=O)[O-])ccc1N2 ; CNc1ccc(-c2nc3ccc(OCCOCCOCCOCCNC(=O)COc4cccc5c4C(=O)N(C4CCC(=O)NC4=O)C5=O)cc3s2)cc1 ; CNc1ccc(-c2nc3ccc(OCCCCCNC(=O)COc4cccc5c4C(=O)N(C4CCC(=O)NC4=O)C5=O)cc3s2)cc1] 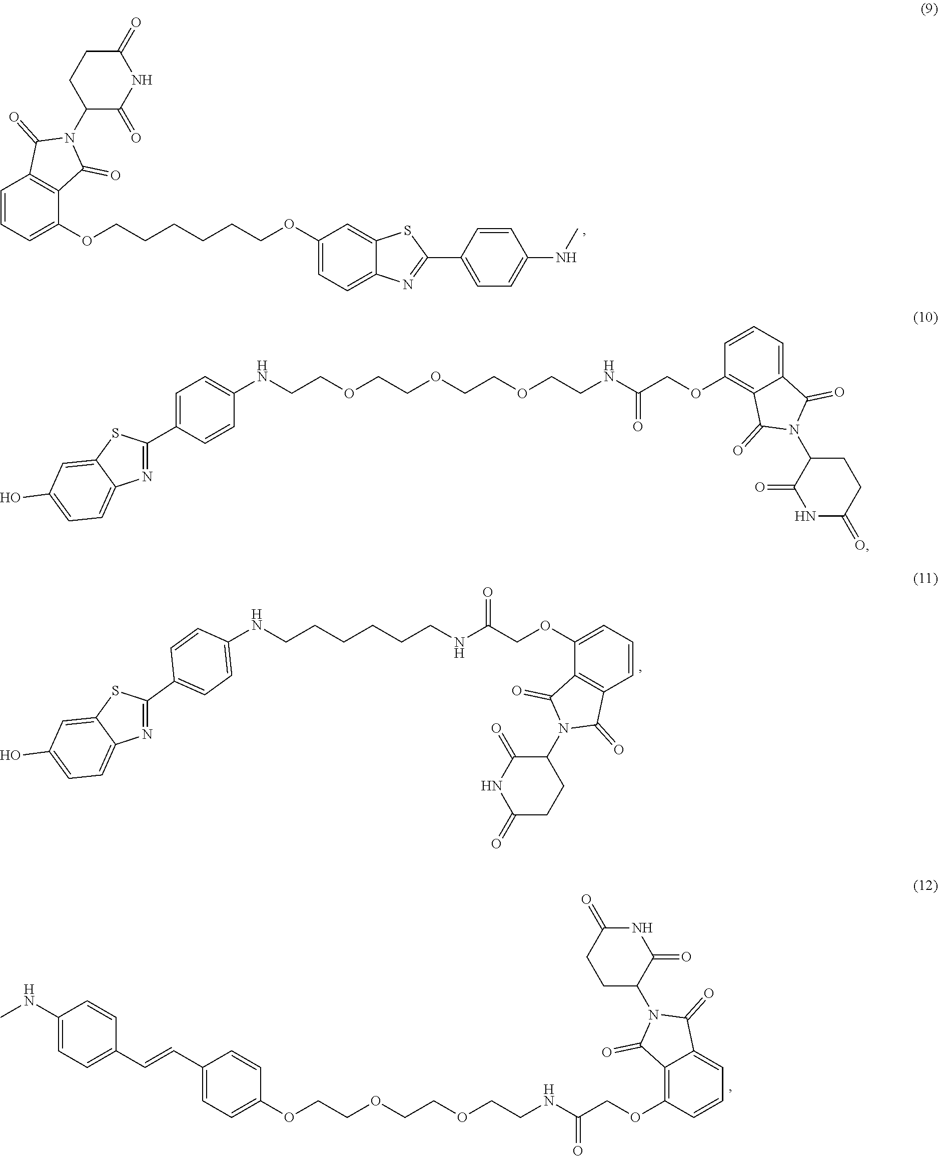 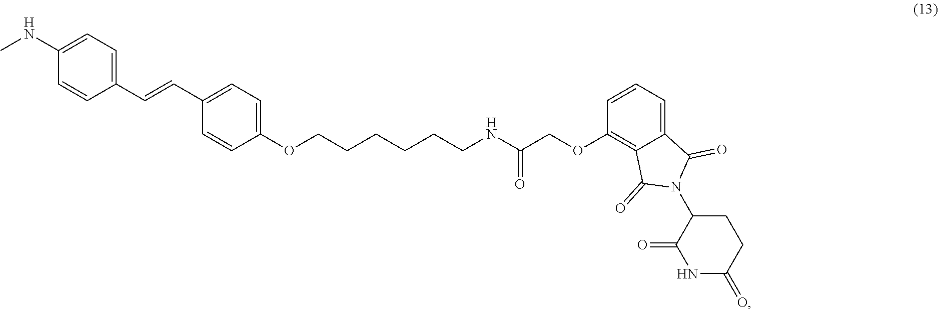 [SMILES: CNc1ccc(/C=C/c2ccc(OCCCCCCNC(=O)COc3cccc4c3C(=O)N(C3CCC(=O)NC3=O)C4=O)cc2)cc1]
or a pharmaceutically acceptable salt or stereoisomer thereof.

18. A pharmaceutical composition, comprising a therapeutically effective amount of the bispecific compound or pharmaceutically acceptable salt or stereoisomer thereof of claim 1, and a pharmaceutically acceptable carrier.

19. The method of treating a neurodegenerative disease or disorder that is characterized or mediated by aberrant activity of α-synuclein, comprising administering to a subject in need thereof a therapeutically effective amount of the bispecific compound or a pharmaceutically acceptable salt or stereoisomer thereof of claim 1.

20. The method of claim 19, wherein the neurodegenerative disease is Parkinson's disease, multiple system atrophy, or dementia with Lewy bodies.

21. (canceled)

22. (canceled)
